(19) United States
(12) Patent Application Publication
(10) Pub. No.: US 2022/0008359 A1
(43) Pub. Date: Jan. 13, 2022

(54) COMBINATION ANTI-CANCER PRODUCTS AND METHODS

(71) Applicant: Ankh Life Sciences Limited, Dublin 2 (IE)

(72) Inventors: Gene H. Zaid, Hutchinson, KS (US); Cameron E. West, Hutchinson, KS (US); Robert Preston Moore, Great Bend, KS (US); Paul Dent, Glen Allen, VA (US)

(73) Assignee: Ankh Life Sciences Limited, Dublin 2 (IE)

(21) Appl. No.: 17/370,675

(22) Filed: Jul. 8, 2021

Related U.S. Application Data
(60) Provisional application No. 63/049,322, filed on Jul. 8, 2020.
(60) Provisional application No. 63/188,202, filed on May 13, 2021.

(51) Int. Cl. A61K 31/12; A61K 39/395; A61K 31/513; A61K 31/11; A61K 31/437; C07K 16/28; A61K 31/519; A61K 31/506; A61K 31/517; A61P 35/00

(52) U.S. Cl. CPC A61K 31/12; A61K 39/3955; A61K 31/513; A61K 31/11; A61K 31/437; A61K 2039/505; A61K 31/519; A61K 31/506; A61K 31/517; A61P 35/00; C07K 16/2827; C07K 16/2818

(57) ABSTRACT

Combination dosage forms of anti-cancer products include components a) and b), where component a) comprises individual amounts of curcumin, harmine, isovanillin, and component b) comprises an agent selected from the group consisting of EGFR inhibitors, CDK 4/6 inhibitors, 5-fluorouracil, checkpoint inhibitors, anti-metabolites, prodrugs thereof, and mixtures thereof. The products may be administered to mammalian subjects suffering from a variety of cancers, to provide a synergistic therapeutic effect.

### CROSS-REFERENCE TO RELATED APPLICATIONS

The present application claims the priority benefit of U.S. Provisional Patent Application Ser. No. 63/049,322, filed Jul. 8, 2020, entitled COMBINATION ANTI-CANCER PRODUCTS AND METHODS, and Ser. No. 63/188,202, filed May 13, 2021, entitled COMBINATION DOSAGE FORMS FOR EGFR INHIBITORS, each of which is incorporated by reference in its entirety herein.

### BACKGROUND OF THE INVENTION

### Field of the Invention

The present invention is broadly concerned with methods and combination dosage forms useful in the treatment of cancer.

### Description of the Related Art

Cancer is a generic term for a large group of diseases that can affect any part of the body. Other terms used are malignant tumors and neoplasms. One defining feature of cancer is the rapid creation of abnormal cells that grow beyond their usual boundaries, and which can then invade adjoining parts of the body and spread to other organs. This process is referred to as metastasis. Metastases are the major cause of death from cancer.

The transformation from a normal cell into a tumor cell is a multistage process, typically a progression from a pre-cancerous lesion to malignant tumors. These changes are the result of the interaction between a person's genetic factors and three categories of external agents, including: physical carcinogens, such as ultraviolet and ionizing radiation; chemical carcinogens, such as asbestos, components of tobacco smoke, aflatoxin (a food contaminant) and arsenic (a drinking water contaminant); and biological carcinogens, such as infections from certain viruses, bacteria, or parasites. Some examples of infections associated with certain cancers include: Viruses: hepatitis B and liver cancer, Human Papilloma Virus (HPV) and cervical cancer, and human immunodeficiency virus (HIV) and Kaposi sarcoma; Bacteria: Helicobacter pylori and stomach cancer; and Parasites: schistosomiasis and bladder cancer.

Aging is another fundamental factor for the development of cancer. The incidence of cancer rises dramatically with age, most likely due to a buildup of risks for specific cancers that increase with age. The overall risk accumulation is combined with the tendency for cellular repair mechanisms to be less effective as a person grows older.

Tobacco use, alcohol use, low fruit and vegetable intake, and chronic infections from hepatitis B (HBV), hepatitis C virus (HCV) and some types of Human Papilloma Virus (HPV) are leading risk factors for cancer in low- and middle-income countries. Cervical cancer, which is caused by HPV, is a leading cause of cancer death among women in low-income countries. In high-income countries, tobacco use, alcohol use, and being overweight or obese are major risk factors for cancer.

The most common cancer treatment modalities are surgery, chemotherapy, and radiation treatments. All of these techniques have significant drawbacks in terms of side effects and patient discomfort. For example, chemotherapy may result in significant decreases in white blood cell count (neutropenia), red blood cell count (anemia), and platelet count (thrombocytopenia). This can result in pain, diarrhea, constipation, mouth sores, hair loss, nausea, and vomiting.

Biological therapy (sometimes called immunotherapy, biotherapy, or biological response modifier therapy) is a relatively new addition to the family of cancer treatments. Biological therapies use the body's immune system, either directly or indirectly, to fight cancer or to lessen the side effects that may be caused by some cancer treatments.

During chemotherapies involving multiple-drug treatments, adverse drug events are common, and indeed toxicities related to drug-drug interactions are one of the leading causes of hospitalizations in the US. Obach, R. S. “Drug-Drug Interactions: An Important Negative Attribute in Drugs.” Drugs Today 39.5 (2003): 308-338. In fact, in any single-month period, one-fifth of all surveyed adults in the USA reported an adverse drug response. Hakkarainen, K. M. et al. “Prevalence and Perceived Preventability of Self-Reported Adverse Drug Events—A Population-Based Survey of 7,099 Adults.” PLoS One 8.9 (2013): e73166. A large-scale study of adults aged 57-85 found that 29% were taking more than five prescription medications and nearly 5% were at risk of major adverse drug-drug interactions. In the field of oncology, a review of over 400 cancer patients determined that 77% were taking drugs that were considered to have a moderately severe potential for adverse drug interactions, and 9% had major adverse drug interactions. Ghalib, M. S. et al. “Alterations of Chemotherapeutic Pharmocokinetic Profiles by Drug-Drug Interactions.” Expert Opin. Drug Metabl. Toxicol 5.2 (2009): 109-130.

Cancer cells are cells that, by definition, grow and divide without normal limitations. The unrestricted cell growth results in tumors, comprised of a variety of cell types. Treatments to fight cancer are frequently successful in killing the typical, differentiated cancer cells that form the majority of a solid tumor, otherwise known as the bulk cells. However even with the best treatment, the cancer may return a few months to years later (Prince, M. E. et al., “Cancer stem cells in head and neck squamous cell cancer.” J. Clin. Oncol. 26.17 (2008):2871-2875). For example, recurrence is frequently the case for pancreatic and head and neck cancer.

Among the known anti-cancer agents are checkpoint inhibitors targeting CTLA4, PD-1, and PD-L1. Another anti-cancer agent is 5-fluorouracil, which is an antimetabolite cytotoxic medicament. While these agents are successful to a greater or lesser degree, any co-administered product which would provide an enhanced or synergistic result would be highly useful in cancer treatments.

U.S. Pat. Nos. 9,907,786 and 10,092,550 describe anti-cancer compositions made up of respective quantities of curcumin, harmine, and isovanillin. Compositions in accordance with the patents are presently in clinical trials and have shown significant promise as anti-cancer compositions.

Despite the immense amount of worldwide research and efforts to stem the tide of cancer and its side effects, the disease in its many manifestations continues to be a huge problem. Moreover, many cancers develop resistance over time to certain standard of care chemotherapeutics. Therefore, any new cancer treatment having a curative affect and/or the ability to ameliorate cancer symptoms and improve the lifestyle of patients is highly significant and important.

### SUMMARY OF THE INVENTION

The present invention is broadly concerned with methods and products useful in the treatment of a broad spectrum of cancers, such as colorectal, breast, brain, lung, lymphatic system, rectal, colon, esophageal, cervical, stomach, and pancreatic cancer. More particularly, the invention is concerned with such methods and products wherein the latter include individual quantities of curcumin, harmine, and isovanillin, together with an agent selected from checkpoint inhibitors, 5-fluorouracil, epidermal growth factor receptor (EGFR) tyrosine kinase inhibitors, CDK 4/6 inhibitors, anti-metabolites, prodrugs thereof, and mixtures thereof. The combination products provide therapeutic synergy for the treatment of mammalian subjects suffering from cancer.

The present invention provides anti-cancer products comprising individual quantities of components a) and b), where component a) comprises individual amounts of curcumin, harmine, isovanillin as defined herein, and component b) comprises an agent selected from the group consisting of checkpoint inhibitors, 5-fluorouracil, epidermal growth factor receptor (EGFR) tyrosine kinase inhibitors, CDK 4/6 inhibitors, anti-metabolites, prodrugs thereof, and mixtures thereof. U.S. Pat. No. 9,907,786 describes a variety of components containing curcumin, harmine, and isovanillin, and uses thereof, and accordingly the '786 is incorporated by reference herein in its entirety.

In certain embodiments, in the components a), the amount of isovanillin is greater than the amounts of harmine and curcumin. The weight ratio of curcumin:harmine:isovanillin in the components a) is usually from about 0.1-25.0:0.1-5:0.1-5, and the isovanillin is present at a level of from about 25-85% by weight, the harmine is present at a level of from about 7-50% by weight, and the curcumin is present at a level of from about 5-40% by weight, all based upon the total weight of the curcumin, harmine, and isovanillin taken as 100% by weight.

The invention also provides methods for treating cancer cells by treating such cells with the combination products, and also methods for treating mammalian subjects (including humans) suffering from cancer, comprising the step of administering to the subjects the described combination products.

### BRIEF DESCRIPTION OF THE DRAWINGS

FIG. 1 is a graph showing the effect on expression levels of programed death ligand 1 (PD-L1) and 2 (PD-L2), major histocompatibility complex class (MHCA), ornithine decarboxylase (ODC), and indoleamine 2,3-dioxygenase (IDO1) in CT26 cells following exposure to 602, 5FU, or the drugs in combination (++).

FIG. 2 is a graph showing the effect on expression levels of PD-L1, PD-L2, MHCA, ODC and IDO1 in human tumor cell lines HCT116, HuCCT1, and KKU following exposure to 602, 5FU, or the drugs in combination (++).

FIG. 3 is a graph showing the effect on expression levels of PD-L1, PD-L2, MHCA, ODC, and ERK2 on both wild type and afatinib resistant NSCLC H1975 cells following exposure to 602 or vehicle control.

FIG. 4 is a graph showing the relative impact on tumor growth over time in an animal tumor model testing GZ17-6.02 and anti-PDl immunotherapy alone and in combination.

FIG. 5 is a survival curve for the animals in the experiment from FIG. 4.

FIG. 6 is a graph showing the relative impact on tumor growth over time in a confirmatory animal study following treatment of the mice with controls (Vehicle (VEH) plus IgG, VEH plus anti-PD-1, and 5 FU plus IgG) and test products (6.02 plus IgG, 6.02 plus anti-PD-1, 6.02 plus 5 FU plus IgG), FIG. 7 shows photographs of relative tumor sizes in the different test groups from the confirmatory animal study.

FIG. 8 is a graph of animal survival versus time developed from data generated in the confirmatory animal study.

FIG. 9A is a graph showing the impact of 602 and 5-FU alone and in combination on the expression of chaperone proteins in GI tumor cell lines HuCCTl and KKU at 4 hours.

FIG. 9B is a graph showing the impact of 602 and 5-FU alone and in combination on the expression of chaperone proteins in GI tumor cell lines HuCCTl and KKU at 8 hours.

FIG. 10A is a graph showing the impact of 602 and 5-FU alone and in combination, including that the decline in chaperone expression requires autophagosome formation, and GRP78 is the most efficacious chaperone at reducing elF2a S51 phosphorylation as tested in HCT116 cells,

FIG. 10B is a graph showing the impact of 602 and 5-FU alone and in combination, including that the decline in chaperone expression requires autophagosome formation, and GRP78 is the most efficacious chaperone at reducing elF2a S51 phosphorylation as tested in HuCCT1 cells.

FIG. 10C is a graph showing the impact of 602 and 5-FU alone and in combination, including that the decline in chaperone expression requires autophagosome formation, and GRP78 is the most efficacious chaperone at reducing elF2a S51 phosphorylation as tested in KKU cells.

FIG. 11A is a graph showing the impact of vehicle control, 602 and 5-FU alone and in combination on autophagy in HCT116 cells as evidenced by increased autophagic vesicle formation (4 hour).

FIG. 11B is a graph showing the impact of vehicle control, 602 and 5-FU alone and in combination on autophagy in HCT116 cells as evidenced by increased autophagic vesicle formation (8 hour).

FIG. 11C is a graph showing the impact of vehicle control, 602 and 5-FU alone and in combination on autophagy in HCT116 T300A cells as evidenced by increased autophagic vesicle formation (4 hour).

FIG. 11D is a graph showing the impact of vehicle control, 602 and 5-FU alone and in combination on autophagy in HCT116 T300A cells as evidenced by increased autophagic vesicle formation (8 hour).

FIG. 11E is a graph showing the impact of vehicle control, 602 and 5-FU alone and in combination on autophagy in HCT116 null cells as evidenced by increased autophagic vesicle formation (4 hour).

FIG. 11F is a graph showing the impact of vehicle control, 602 and 5-FU alone and in combination on autophagy in HCT116 null cells as evidenced by increased autophagic vesicle formation (8 hour).

FIG. 11G is a graph showing the impact of vehicle control, 602 and 5-FU alone and in combination on autophagy in HCT116 p⁵³ null cells as evidenced by increased autophagic vesicle formation (4 hour).

FIG. 11H is a graph showing the impact of vehicle control, 602 and 5-FU alone and in combination on autophagy in HCT116 p53 null cells as evidenced by increased autophagic vesicle formation (8 hour).

FIG. 12A shows the results from cell viability testing in different HCT116 cell lines subjected to vehicle control, 602 and 5-FU alone and in combination at 24 hours.

FIG. 12B shows the results from cell viability testing in HCT116 ATG16L1 null cells subjected to vehicle control, 602 and 5-FU alone and in combination at 24 hours, showing that ATG16L1 isoform expression and death receptor signaling play key roles in mediating 602 lethality.

FIG. 13A is a graph of cell viability testing in KKU cells knocked down for various indicated proteins and treated with vehicle control, 602 (2 μM), 5FU (50 μM) or the drugs in combination for 24 hours.

FIG. 13B is a graph of cell viability testing in KKU cells knocked down for various indicated proteins and treated with vehicle control, 602 (2 μM), 5FU (50 μM) or the drugs in combination for 24 hours.

FIG. 13C is a graph of cell viability testing in KKU cells knocked down for various indicated proteins and treated with vehicle control, 602 (2 μM), 5FU (50 μM) or the drugs in combination for 24 hours.

FIG. 14A is a graph of cell viability testing in HCT116 cells knocked down for various indicated proteins and treated with vehicle control, 602 (2 μM), 5FU (50 μM) or the drugs in combination for 24 hours.

FIG. 14B is a graph of cell viability testing in HCT116 cells knocked down for various indicated proteins and treated with vehicle control, 602 (2 μM), 5FU (50 μM) or the drugs in combination for 24 hours.

FIG. 15A is a graph showing the impact of knockdown of elF2-alpha on the drug-induced expression of indicated proteins in HCT116 cells.

FIG. 15B is a graph showing the impact of knockdown of elF2-alpha on the drug-induced expression of indicated proteins in CT26 cells.

FIG. 16A is a graph of expression levels in HCT116 cells knocked for ATM expression treated with vehicle control, 602 (2 μM), 5FU (50 μM) or the drugs in combination for 1 hour.

FIG. 16B is a graph of expression levels in HCT116 cells knocked for ATM expression treated with vehicle control, 602 (2 μM), 5FU (50 μM) or the drugs in combination for 1 hour.

FIG. 17A is a graph showing changes in ATM phosphorylation observed in HuCCT1 cell lines.

FIG. 17B is a graph showing changes in ATM phosphorylation observed in KKU cell lines.

FIG. 18 is a graph showing impact of 602, 5FU or the combination (++) combination on cellular signaling and protein expression, Histone Deacetylase (HDAC) expression in CT26 cells.

FIG. 19 shows the effect of treatment with vehicle control, 602, palbociclib, or combination on different breast cancer cells (A) MCF7, (B) BT483, and (C) ZR 75-1 after 24 hours.

FIG. 20A is a graph showing the impact of vehicle control, 602 and palbociclib alone and in combination on autophagy in ZR 75-1 breast cancer cells, as evidenced by increased autophagic vesicle formation.

FIG. 20B is a cell viability assay on ZR 75-1 cells with knockdown of various proteins and treated with vehicle control or 602/palbociclib combination at 24 hours.

FIG. 20C is a cell viability assay on ZR 75-1 cells with knockdown of various proteins and treated with vehicle control or 602/palbociclib combination at 24 hours.

FIG. 20D is a cell viability assay on ZR 75-1 cells with knockdown of various proteins and treated with vehicle control or 602/palbociclib combination at 24 hours.

FIG. 20E is a graph showing the impact of vehicle control, 602 and palbociclib alone and in combination on autophagy in MCF7 breast cancer cells, as evidenced by increased autophagic vesicle formation.

FIG. 20F is a cell viability assay on MCF7 cells with knockdown of various proteins and treated with vehicle control or 602/palbociclib combination at 24 hours.

FIG. 20G is a cell viability assay on MCF7 cells with knockdown of various proteins and treated with vehicle control or 602/palbociclib combination at 24 hours.

FIG. 20H is a cell viability assay on MCF7 cells with knockdown of various proteins and treated with vehicle control or 602/palbociclib combination at 24 hours.

FIG. 21 is a graph showing interaction of 602 with erlotinib, afatinib and osimertinib to kill H1975 and H1650 cells that express mutant activated ERBB1 proteins.

FIG. 22A is a graph showing interaction of 602 with erlotinib, afatinib and osimertinib to kill osimertinib-resistant H1975 and H1650 cells.

FIG. 22B is a graph showing interaction of 602 and pemetrexed to kill osimertinib-resistant H1975 and H1650 cells.

FIG. 22C is a graph showing interaction of 602 and pemetrexed to kill other NSCLC cell lines.

FIG. 23A is a graph showing the interaction of 602 and pemetrexed on the ability of H1975 cells or afatinib-resistant (AR) H1975 cells to form autophagosomes.

FIG. 23B is a graph showing the interaction of 602 and pemetrexed on the ability of afatinib-resistant H1975 cells to form autophagosomes after drug exposure at 4 or 8 hours, showing a significant reduction via knock down of eIF2a, ATM or AMPKa or by expression of activated mTOR or activated STAT3.

FIG. 23C is a graph showing the interaction of 602 and pemetrexed to cause autophagosome formation in the osimertinib-resistant (OR) H1975 cells.

FIG. 24A is a graph showing alterations in cell signaling, autophagy and viability after treating A549 NSCLC cells that express a mutant K-RAS protein with the vehicle control, 602, pemetrexed, or drugs in combination.

FIG. 24B is a graph showing the interaction of 602 and pemetrexed to alter cell signaling, increase autophagosome formation and kill via toxic autophagy in A549 NSCLC cells that express a mutant K-RAS protein.

FIG. 24C is a graph of viability data after treatment with vehicle control or 602 and pemetrexed.

FIG. 25 shows graphs of viability data in several cell lines showing the ability of vehicle control or 602 and pemetrexed to kill osimertinib-resistant cells.

FIG. 26 is a graph of control studies presented to confirm on-target specificity of the siRNAs, primary antibodies and the phospho-specific antibodies to detect both total protein levels and phosphorylated levels of proteins.

FIG. 27A is a graph showing the interaction of 602 and osimertinib on the ability of H1975 cells or afatinib-resistant (AR) H1975 cells to form autophagosomes at 4 or 8 hours.

FIG. 27B is a graph showing that knock down of ATM, AMPKa, eIF2a or expression of activated mTOR or activated STAT3 significantly suppressed autophagosome formation and autophagic flux in HCC827 cells or erlotinib-resistant (ER) HCC827 cells.

FIG. 27C is a graph showing knock down of ATM, AMPKa, eIF2a or expression of activated mTOR or activated STAT3 significantly suppressed autophagosome formation and autophagic flux in afatinib-resistant (AR) H1975 cells at 4 or 8 hours.

FIG. 28 is a graph from cell viability studies showing knock down of [BAX+BAK], Beclin1, ATG5 or FADD significantly reduced cell killing by 602 and osimertinib.

FIG. 29 is a graph showing that total levels of GSH and the GSH:GSSG ratio were not significantly altered by 602 treatment over 12 hours.

FIG. 30 is a graph showing alterations in cell signaling, autophagy and viability data by treating A549 NSCLC cells which express a mutant K-RAS protein and erlotinib-resistant HCC827 cells with the drug combination.

### DETAILED DESCRIPTION OF THE PREFERRED EMBODIMENTS

In more detail, Arum (A.) palaestinum, a member of the Araceae family of plants, is a foundation of traditional medicine in treating disorders ranging from stomach upset to cancer. A. palaestinum extracts containing isovanillin, were combined with other plant extracts and studied. Additional work identified a three-component mixture which showed clear preclinical synergy between the three components, isovanillin identified in A. palaestinum, harmine identified in Peganum harmala and curcumin identified in Curcuma longa.

### The Curcumin-Harmine-Isovanillin Compositions

Curcumin (diferuloylmethane, 1,7-bis(4-hydroxy3-mcthoxyphenyl)-1,6-heptadiene-3,5-dione) is a symmetrical diaryl heptanoid. It occurs as a part of a curcuminoid plant extract containing curcumin, demethoxycurcumin, and bis-demethoxycurcumin.

| | |
|---|---|
| CAS Number: 458-37-7 | 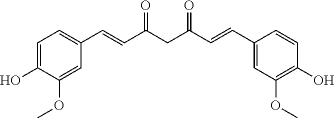 [SMILES: COc1cc(/C=C/C(=O)CC(=O)/C=C/c2ccc(O)c(OC)c2)ccc1O] |
| Molecular | C21H206 |
| Formula: | |
| Molecular | 368.39 |
| Weight: | |

It exists in solution as an equilibrium mixture of the symmetrical dienone (diketo) and the keto-enol tautomer; the keto-enol form is strongly favored by intramolecular hydrogen bonding.

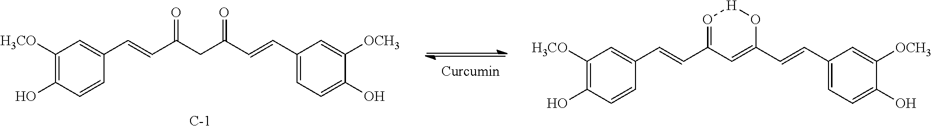

Curcumin contains two aryl rings separated by an unsaturated 7-carbon linker having a symmetrical β-diketone group (as used herein, “β-diketone” embraces both tautomeric forms, namely the diketo and enol forms). The aryl rings of curcumin contain a hydroxyl group in the para position and a methoxy group in the meta position.

Harmine (7-methoxy-1-methyl-9H-pyrido[3,4-b]indole) is a methoxy methyl pyrido indole belonging to the β-carboline family of compounds.

| CAS Number: 441-51-3 | 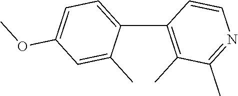 [SMILES: COc1ccc(-c2ccnc(C)c2C)c(C)c1] |
|---|---|
| Molecular Formula: | C13H12N2O |
| Molecular Weight: | 212.25 |

The chemical structure of harmine, 1-methyl-7-methoxy-β-carboline, is shown as follows:

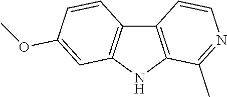 [SMILES: COc1ccc2c(c1)[nH]c1c(C)nccc12]

Isovanillin (CAS #621-59-0) is a phenolic aldehyde vanillin isomer, and has the molecular formula C8H8O3. The vanillin compound(s) useful in the invention are phenyl aldehydes, and one family of such compounds have the structure

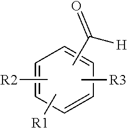

where R1 is selected from the group consisting of OH, H, C1-C4 alkoxy groups, F, Cl, Br, I, N, and NO2, and R2 and R3 are independently selected from the group consisting of H, OH, and C1-C4 alkoxy groups, it being understood that the aldehyde group and R1, R2, and R3 can be located at any position around the phenyl ring.

Certain specific vanillin compounds are vanillin, isovanillin, orthovanillin, and include the following exemplary vanillin compounds:

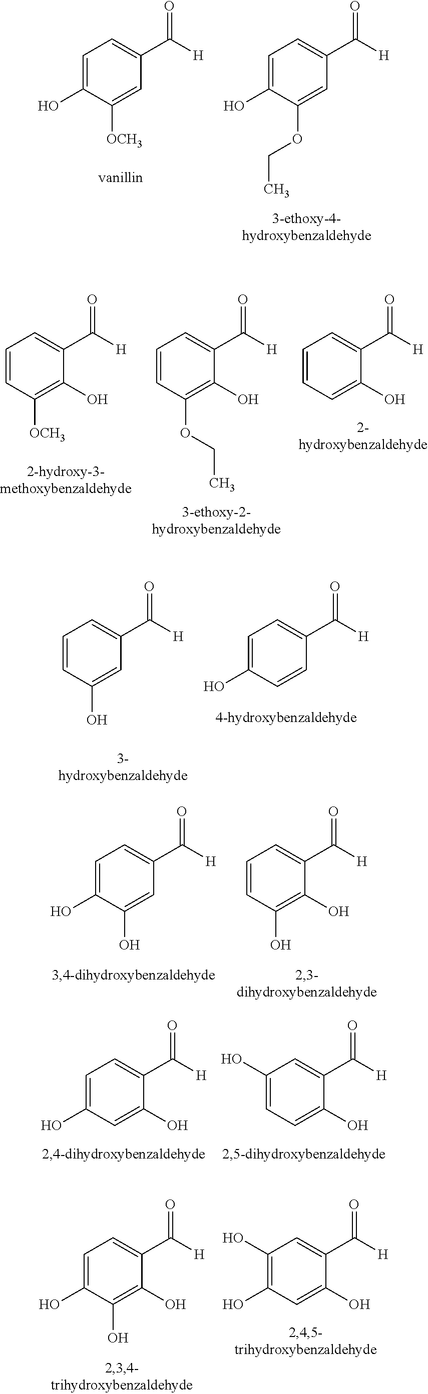

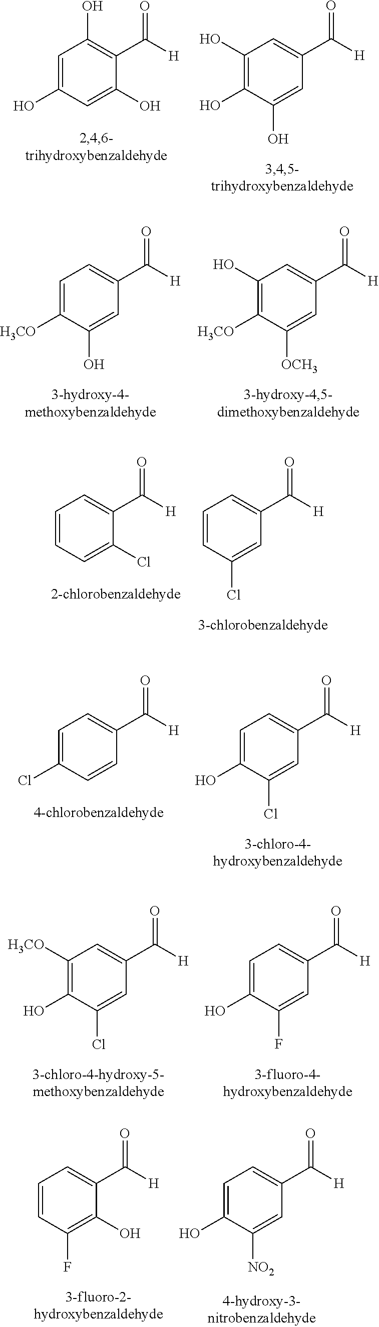

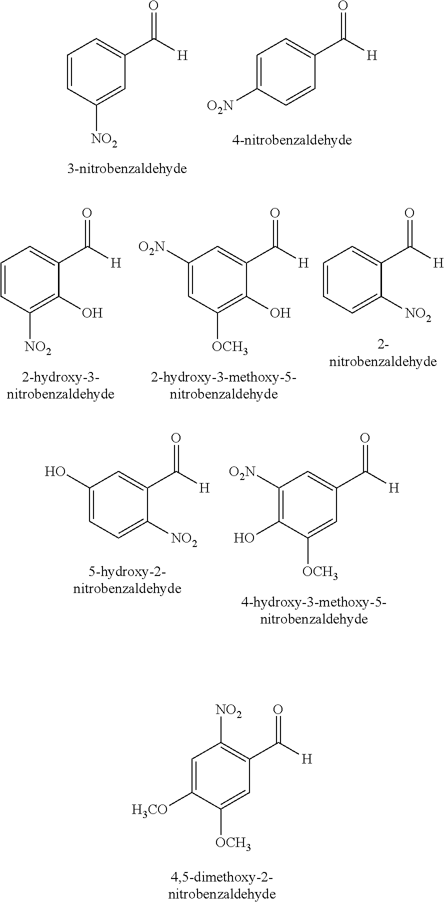

Thus, as used herein, unless otherwise dictated by the context, “curcumin,” “harmine,” and “isovanillin” respectively refer to the above-identified compounds as well as the isomers, tautomers, derivatives, solvates, degradation products, metabolites, esters, metal complexes (e.g., Cu, Fe, Zn, Pt, V), prodrugs, and pharmaceutically acceptable salts thereof. As used herein, a derivative is a compound that can be imagined to arise or actually be synthesized from a parent compound by replacement of one atom with another atom or a group of atoms while at least maintaining the desired degree of pharmacological activity of the parent compound. Similarly, pharmaceutically acceptable salts with reference to the components of the composition means salts which are pharmaceutically acceptable, e.g., salts which are useful in preparing pharmaceutical compositions that are generally safe, non-toxic, and neither biologically nor otherwise undesirable and are acceptable for human pharmaceutical use, and which possess the desired degree of pharmacological activity. Such pharmaceutically acceptable salts may include acid addition salts formed with organic or inorganic acids, and base addition salts. In preferred practice, the individual components are naturally or synthetically derived, and should have purities of at least about 90% by weight, and most preferably at least about 98% by weight.

In the three-component compositions, isovanillin would normally be the predominant ingredient on a weight basis, with harmine and curcumin being present in lesser amounts on a weight basis. Generally, isovanillin should be present at a level of at least about three times (more preferably at least about five times) greater than that of each of harmine and curcumin, again on a weight basis. The as-added amounts of the components should give weight ratios of from about 0.1-25.0:0.1-5:0.1-5 (isovanillin:harmine:curcumin), and more preferably from about 10:1.7:0.85. In terms of amounts of the three components, isovanillin should be present at a level of from about 25-85% by weight, harmine at a level of from about 7-50% by weight, and curcumin at a level of from about 5-40% by weight, all based upon the total weight of the three ingredients taken as 100% by weight.

“Pharmaceutically acceptable salts” with reference to the components means salts of the components which are pharmaceutically acceptable, i.e., salts which are useful in preparing pharmaceutical compositions that are generally safe, non-toxic, and neither biologically nor otherwise undesirable and are acceptable for human pharmaceutical use, and which possess the desired degree of pharmacological activity. Such pharmaceutically acceptable salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, and Use, P. H. Stahl & C. G. Wermuth eds., ISBN 978-3-90639-058-1 (2008).

### GZ17-6.02

One three-component active agent combination in accordance with the invention is referred to as “GZ17-6.02,” and used interchangeably herein with “6.02” or “602”. This combination comprises 77% by weight of 98% pure solid synthetic isovanillin, 13% by weight of 99% pure solid synthetic harmine, and 10% by weight of a commercially available solid curcumin product containing 99.76% by weight curcuminoids, namely 71.38% curcumin, 15.68% demethoxycurcumin, and 12.70% bisdemethoxycurcumin. The solids (powders) are thoroughly mixed together to complete the preparation. Thus, the three-component active agent combination consists of a mixture of individual quantities of normally highly purified curcumin, harmine, and isovanillin components at ratios of approximately 0.1-25:0.1-5:0.1-5 (isovanillin:harmine:curcumin). Each such component may be made up of one or more isovanillin, harmine, and/or curcumin compounds. Generally, it is preferred that the isovanillin component is the preponderant component in the composition on a weight basis, with the harmine and curcumin components being present in lesser amounts on a weight basis. Still further, the isovanillin component may be present at a level of at least three times (more preferably at least five times) greater than that of each of the harmine and curcumin components. In terms of amounts of the three components, the isovanillin component should be present at a level of from about 25-85% by weight, the harmine component should be present at a level of from about 7-50% by weight, and the curcumin component should be present at a level of from about 5-40% by weight, all based on the total weight of the three components taken as 100% by weight.

The single most preferred GZ17-6.02 active agent combination, and that tested in the examples, was made by dispersing relative quantities of solid synthetic isovanillin (771 mg, 98% by weight purity), synthetic harmine (130.3 mg, 99% by weight purity), and a commercially available curcumin product derived by the treatment of turmeric (98.7 mg, containing 99.76% by weight curcuminoids, namely 71.38% curcumin, 15.68% demethoxycurcumin, and 12.70% bisdemethoxycurcumin), at a weight ratio of 771:130.3:98.7 (isovanillin:harmine:curcumin product).

### Checkpoint Inhibitors

Checkpoint inhibitor therapy is a form of cancer immunotherapy targeting immune checkpoints, which are key regulators of the immune system that when stimulated can dampen the immune response to an immunologic stimulus. Some cancer cells can protect themselves from attack by simulating immune checkpoint targets. Checkpoint therapy serves to block inhibitory checkpoints, thereby restoring immune system function.

Currently approved checkpoint inhibitors target the molecules CTLA4, PD-1, and PD-L1 (programmed death ligand 1). PD-1 is the transmembrane program cell death 1 protein (also called PDCD1 and CD279) which interacts with PD-L1. PD-L1 on cancer cell surfaces binds to PD-1 on an immune cell surface, which inhibits immune cell activity. Among PD-L1 functions is a key regulatory role on T-cell activities. It is postulated that cancer-mediated upregulation on the cell surface may inhibit T-cells that otherwise may attack cancer cells. Antibodies that bind to either PD-1 or PD-L1 and therefore block this interaction allow the T-cells to attack the cancer cells. Immunotherapy with PD-I blockage or PD-L1 blockage have been reported for use in many cancers, including melanoma, non-small cell lung cancer, renal cell carcinoma, ovarian cancer, lymphoma, and the like.

The following approved checkpoint inhibitors and their respective targets are:

| Name | Target |
|---|---|
| Ipilimumab | CTLA-4 |
| Nivolumab | PD-1 |
| Pembrolizumab | PD-1 |
| Camrelizumab | PD-L1 |
| Atezolizumab | PD-L1 |
| Avelumab | PD-L1 |
| Durvalumab | PD-L1 |
| Cemiplimab | PD-1 |

Ipilimumab (brand name “YERVOY®”) is approved, inter alia, for the treatment of melanoma. Nivolumab (brand name “OPDIVO®”) is approved, inter alia, to treat melanoma, lung cancer, kidney cancer, bladder cancer, head and neck cancer, and Hodgkin's Lymphoma. Pembrolizumab (brand name “KEYTRUDA®”) is approved, inter alia, to treat melanoma and lung cancer. Atezolizumab is approved, inter alia, for treatment of bladder cancer. Spartalizumab (PDR001) is currently being developed as a treatment for solid tumors and lymphomas. Other modes of enhancing immunotherapy include targeting of so-called intrinsic checkpoint blockades, for example, chromogenic in situ hybridisation (CISH). Given the immunotherapy importance of checkpoint inhibitors, it is anticipated that other types of inhibitors will be developed in the future.

As used herein, a “checkpoint inhibitor” shall mean any agent of chemotherapy and/or immunotherapy which targets one or more immune checkpoints to lessen or block inhibitory checkpoints in order to promote immune system function against cancer cells. Non-limiting examples include approved or investigational checkpoint inhibitors such as anti-programmed cell death protein 1/programmed cell death ligand 1(PD-1/PD-L1), anti-cytotoxic T lymphocyte-associated antigen-4 (CTLA-4), lymphocyte activation gene-3 (LAG3), T cell immune globulin and mucin-domain containing-3 (TIM-3), T cell immunoglobulin and ITIM domain (TIGIT), V-domain Ig suppressor of T cell activation (VISTA), and the like.

### Anti-Metabolites

5-Fluorouracil (5 FU) is an anti-metabolite drug used to treat cancers of the skin, breast, rectum, colon, esophagus, cervix, stomach, and pancreas, and is provided under brand names “CARAC®,” “TOLAK®,” “EFUDEX®,” “ADRUCIL®,” and “FLUOROPLEX®.” ADRUCIL® is in the form of an IV drug, while the others are commercialized as topical creams. 5 FU is also being studied for the treatment of other conditions and type of cancers. Prodrugs of 5 FU are also contemplated, such as Capecitabine (“XELODA®”), which is an approved orally administered compound that is metabolically converted to from capecitabine to 5FU in the tumor.

Pemetrexed (“ALIMTA®”) is an anti-metabolite drug used to treat malignant mesothelioma, as well as locally advanced or metastatic nonsquamous non-small cell lung cancer. It is given as an infusion into the vein (intravenous, IV).

### EGFR Inhibitors

EGFR inhibitors include two main classes: tyrosine kinase inhibitors (TKIs) and monoclonal antibodies (mabs). The TKIs are oral drugs and the mabs are IV drugs but both affect EGFR albeit by different mechanisms. TKIs include, without limitation, Erlotinib (TARCEVA®)*, Osimertinib (TAGRISSO®)*, Gefitinb (IRESSA®), Afatinib (GILOTRIF®)*, and Dacomitinib (VIZIMPRO®). Mabs include Cetuximab (ERBITUX®), Panitumuab (VECTIBIX®), and Necitumumab (PORTRAZZA®).

It may be important to recognize that other drugs may affect EGFR but only affect EGFR as off target interactions. An example from this list is Afatinib, which is considered an EGFR inhibitor but also effects HER2 and HER4 and alternatively some HER2 TKIs affect EGFR off target. These off-target effects occur because EGFR, HER2, PI3K, and HER4 are all part of the ERBB family of receptors (ERBB1=EGFR, ERBB2=HER2, ERBB3=PI3K, ERBB4=HER4) and are very closely related. As such, it will be very difficult to specifically point out all active agents or therapies that effect EGFR as other drugs may affect EGFR off-target, but the current list is of the drugs specifically indicated to treat EGFR mutations. The present disclosure also concerns prodrugs and metabolites of the foregoing active agents.

### CDK 4/6 Inhibitors

Palbociclib (brand name IBRANCE®) is an orally available cyclin-dependent kinase (CDK) inhibitor with potential antineoplastic activity. Palbociclib selectively inhibits cyclin-dependent kinase 4 (CDK4) and 6 (CDK6), thereby inhibiting retinoblastoma (Rb) protein phosphorylation early in the G1 phase leading to cell cycle arrest. This suppresses DNA replication and decreases tumor cell proliferation. CDK4 and 6 are serine/threonine kinases that are upregulated in many tumor cell types and play a key role in the regulation of cell cycle progression. The compound is currently considered a front line treatment of certain patients with HR+/HER2-Metastatic Breast Cancer. Other inhibitors in this class include Ribociclib (KISQALI®) and Abemaciclib (VERZENIO®).

Palbociclib is also currently being studied in treating patients with Rb positive solid tumors, non-Hodgkin's lymphoma, or histiocytic disorders with activating alterations (mutations) in cellcycle genes that have spread to other places in the body and have come back or do not respond to treatment. Palbociclib may stop the growth of cancer cells by blocking activity of two closely related enzymes known to promote tumor cell growth.

### The Combination Products of the Invention

As indicated above, the combination products of the invention comprise a) individual quantities of curcumin, harmine, and isovanillin as a mixture, and b) an agent selected from the group consisting of EGFR inhibitors, CDK 4/6 inhibitors, checkpoint inhibitors, anti-metabolites, 5-fluorouracil, prodrugs thereof, and mixtures thereof. As used herein, “combination” or “in combination” are intended to embrace products wherein the individual ingredients are physically intermixed as combined unit dosage forms, and to situations where the individual ingredients are separately administered via the same or different administration routes to a mammalian subject over periods of time, either simultaneously or as separate sequential administrations, unless otherwise indicated by the context. For example, component a) may be administered to the subject orally while component b) is administering intravenously, with both components being administered simultaneously or as separate sequential administrations but nonetheless being still considered a combination therapeutic treatment. Likewise, component a) and component b) could both be administered orally (or intravenously, etc.), but as separate unit dosage forms either simultaneously or as separate sequential administrations as part of the same combination therapeutic treatment. Similarly, component a) and component b) could be pre-mixed or combined into a single combined unit dosage form that is administered to the patient for the combination therapeutic treatment. A clinician or researcher may determine the appropriate administration protocol based upon the particular agents used in the combination therapy. In any case, component a) and component b) are used in combination in embodiments of the invention to augment or improve the anti-cancer effects of the individual components as compared to when either component is used alone as part of a cancer treatment protocol.

Additional ingredients may be included with the chemotherapeutic agents of the invention for administration to the subject. Such additional ingredients include, other active agents, preservatives, buffering agents, salts, carriers, excipients, diluents, or other pharmaceutically acceptable ingredients. The basic parts a) and b) of the combination products may also include pharmaceutically acceptable carriers, diluents, excipients, vehicles, and the like, such as sterile water, saline, polyalkylene glycols, vegetable oils, pharmaceutically acceptable polymers, and mixtures thereof in which the active agents may be dispersed, dissolved, or suspended. In one or more embodiments, the curcumin/harmine/isovanillin combination is administered in connection with the intake of food or nutrient substances, particularly fats, lipids, triglycerides (particularly medium chain triglycerides), etc. to enhance the bioavailability of the components. For example, the curcumin/harmine/isovanillin combination may be suspended or dissolved in PEPTAMEN® or other fat-containing liquid before administration, or co-administered with such fatty substances. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would be selected to minimize any degradation of the compound or other agents and to minimize any adverse side effects in the subject. Pharmaceutically-acceptable ingredients include those acceptable for veterinary use as well as human pharmaceutical use, and will depend on the route of administration. For example, compositions suitable for administration via injection are typically solutions in sterile isotonic aqueous buffer. Exemplary carriers include aqueous solutions such as normal (n.) saline (˜0.9% NaCl), phosphate buffered saline (PBS), sterile water/distilled autoclaved water (DAW), various oil-in-water or water-in-oil emulsions, as well as dimethyl sulfoxide (DMSO) or other acceptable vehicles, and the like.

In use, therapeutically effective amounts of the combined products of the invention are administered to a mammalian subject in need thereof for a therapeutically effective amount of time. As used herein, a “therapeutically effective” amount refers to the dosage amount and/or duration that will elicit the biological or medical response of a tissue, system, or subject that is being sought by a researcher or clinician, and in particular elicit some desired therapeutic effect as against the cancer cells by slowing and/or inhibiting activity, growth, or metastasis of the cancer cells and/or associated tumor. One of skill in the art recognizes that an amount or duration may be considered therapeutically “effective” even if the condition is not totally eradicated or prevented, but it or its symptoms and/or effects are improved or alleviated partially or inhibited from worsening in the subject. Such therapeutically effective dosages and durations may comprise a single unit dosage or, more usually, periodic (e.g., daily or weekly) administration of lower dosages over time. In some embodiments, upon administration, the prodrug mechanism of action entails enzyme-mediated, chemical, or spontaneous degradation or hydrolysis that converts the prodrug into an active metabolite (in some cases involving one or more intermediate compounds).

Advantageously, administration of such therapeutically effective amounts achieves an unexpected therapeutic synergy. This means that the therapeutic combinations of the invention exhibit a joint action where one of the components a) or b) supplements or enhances the action of the other component to produce an effect greater than that which may be obtained by use of individual components in equivalent quantities. Generally, the components a) and b) working together produce a therapeutic effect greater than the sum of their individual effects. Without wishing to be bound by theory, in some aspects, component a) enhances the action of component b) by modulating super enhancers and/or changing epigenetic, oncogene, and tumor suppressor gene expression (up or down) in a manner benefiting the mechanism of action of component b) thereby increasing overall treatment efficacy. Also of great importance is the ability or potential ability of component a) to affect not only tumor cells themselves but also the tumor microenvironment in which cancer manipulates the cellular and molecular interactions of the malignant and non-malignant cells to cause further gene mutation, dysregulation, cellular proliferation, and immune response escape by the cancer cells. The tumor microenvironment includes normal cells, molecules, and blood vessels that surround and feed a tumor cell. A tumor can change its microenvironment, and the microenvironment can affect how a tumor grows and spreads. By affecting not only tumor cells but also the tumor microenvironment, component a) has the ability to enhance the therapeutic effect of component b) in an unexpected manner. Thus, the combination therapies provide novel and unexpected ways to directly or indirectly disrupt cancer cell interactions and the microenvironment to fight cancer.

The components a) and b) may be individually administered in any convenient manner, such as by oral, rectal, nasal, ophthalmic, parenteral (including intraperitoneal, intravesical, gastrointestinal, intrathecal, intravenous, cutaneous (e.g., dermal patch), subcutaneous (e.g., injection or implant), or intramuscular) administrations. The dosage forms of the invention may be in the form of liquids, gels, suspensions, solutions, or solids (e.g., tablets, pills, or capsules). Moreover, therapeutically effective amounts of the agents of the invention may be co-administered with other chemotherapeutic agent(s), where the two products are administered substantially simultaneously (e.g., as part of the same dosage form, or separately but at nearly same time within less than an hour of each other) or in any sequential manner. The term “unit dosage form” refers to a physically discrete unit suitable as a unitary dosage for human or animal use. Each unit dosage form may contain a predetermined amount of the component a) and/or component b) in the carrier calculated to produce a desired effect. In certain embodiments, component a) is orally administered in powder or liquid capsule form, whereas component b) is administered by parenteral injection, for example substantially simultaneously or sequentially.

Dosage levels administered to mammalian subjects using the combination products of the invention are quite variable owing to factors such as the subject's age, subject's physical condition, the type of condition(s) being treated (e.g., specific cancer(s)), and the severity of the conditions. Determination of proper dosage levels can readily be determined by those skilled in the art.

The combination products of the invention are particularly useful for the treatment of mammalian subject suffering from a variety of cancers wherein component a) augments or enhances the anti-cancer activity of component b); and/or wherein component b) augments or enhances the anti-cancer activity of component a); and/or wherein component a) and component b) work in synergy to generate an anti-cancer effect on the cancer cells being treated (e.g., inhibition of cancer cell growth, increase in cancer cell death, etc.). The combination products can be used to treat, for example, cancers of the skin, breast, rectum, colon, esophagus, cervix, stomach, and pancreas, and especially colorectal cancers. It will be appreciated that therapeutic and prophylactic methods described herein are applicable to humans as well as any suitable animal, including, without limitation, dogs, cats, and other pets, as well as, rodents, primates, horses, cattle, pigs, etc. The methods can be also applied for clinical research and/or study.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration, and nothing therein should be taken as a limitation upon the overall scope of the invention.

### Example 1

Preliminary in vitro testing established that GZ17-6.02 affected various biological functions that mitigate or decrease immune checkpoint blockade in various cell lines and tissue types. These effects were observed in cell lines from tissues with favorable response rates, “hot” tumors, and from tissues with little to no response to immunotherapy, “cold” tumors. In light thereof, further in vitro tests were conducted to determine whether changes in HDAC expression and/or localization altered protein expression in these tumor cells, specifically putative biomarkers for immunotherapy response, namely PD-L1 (Programmed Death Ligand 1), ODC (Ornithine Decarboxylase), IDO1 (Indoleamine 2,3-Dioxygenase), and MHCA (Major Human Histo-Compatibility Complex Class 1A).

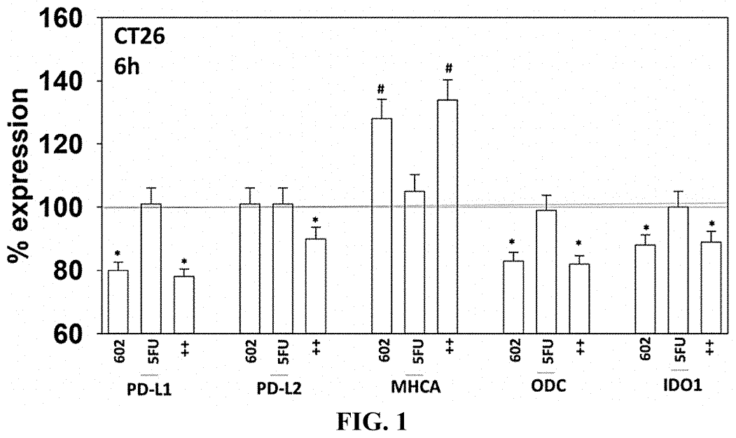

In vitro, 602 was observed to change the levels of various proteins that generally effect a cancer's ability to avoid immune detection and destruction. Cells from the CT26 colorectal cancer cell line were treated with vehicle control or with 602 (final curcumin concentration 2.0 μM) in the presence or absence of 5FU (50 μM). Cells were fixed in situ 6 hours after drug exposure. Fixed cells were permeabilized, blocked, and stained overnight with the indicated validated primary antibodies. The following morning, the plates were washed, and secondary antibodies were added carrying either 488 nm (green) or 594 nm (red) fluorescent tags. Images of the stained cells were brought into focus and then the Hermes WiScan internal software used to define via random selection the staining intensity of at least 120 cells per well/condition. The graphical data presented in FIG. 1 are the normalized amount of fluorescence set at 100% comparing intensity values for vehicle control and 602/5FU treatments (n=3+/−standard error [SD]). *p<0.05 less than vehicle control value. These changes in protein expression demonstrate 602's ability to affect protein expression used by cancer to escape immune detection and destruction compared to standard chemotherapy 5-fluorouracil.

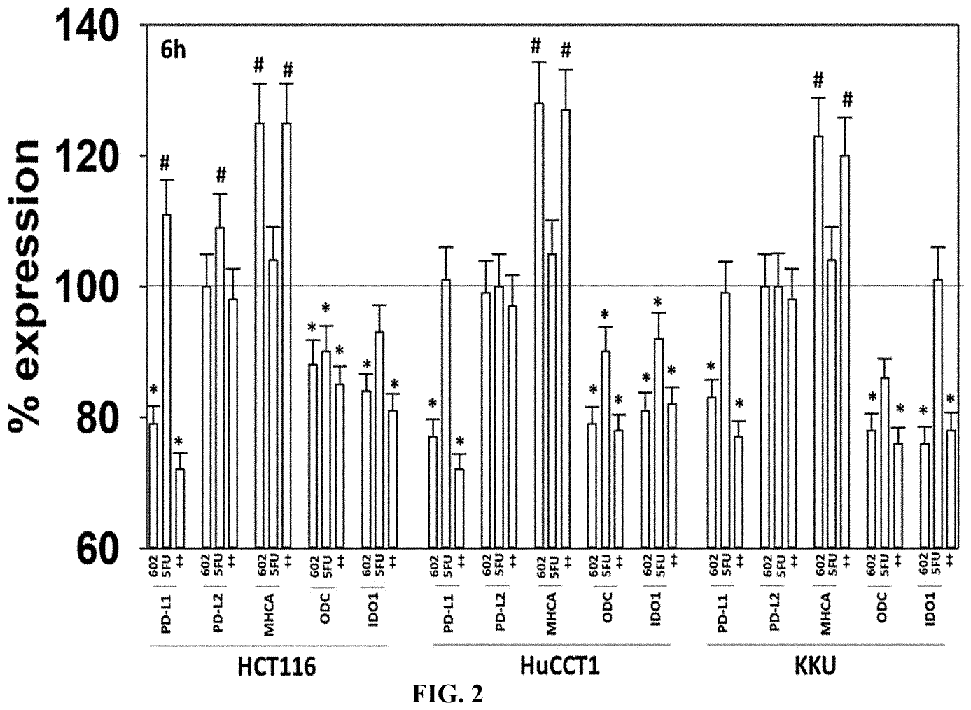

Additionally, exposure to 602 reduced PD-L1, ODC and IDO1 levels and increased MHCA expression other human cell lines. Cells were treated with vehicle control or with 602 (final curcumin concentration 2.0 μM) in the presence or absence of 5FU (50 μM). Cells were fixed in situ 6 h after drug exposure. Fixed cells were permeabilized, blocked and stained overnight with the indicated validated primary antibodies. The following morning, the plates were washed, and secondary antibodies added carrying either 488 nm (green) or 594 nm (red) fluorescent tags. Images of the stained cells were brought into focus and then the Hermes WiScan internal software used to define via random selection the staining intensity of at least 120 cells per well/condition. The graphical data presented in FIG. 2 show the normalized amount of fluorescence set at 100% comparing intensity values for vehicle control and 602/5FU treatments (n=3+/−SD). * p<0.05 less than vehicle control value.

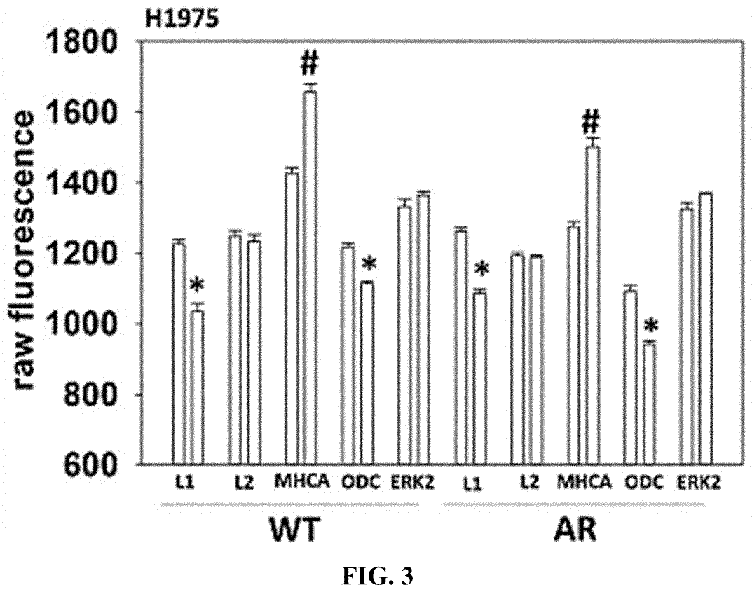

602 exposure reduced the expression of PD-L1 and ODC and increased MHCA expression in both wild type and afatinib resistant NSCLC H1975 cell lines, as shown in FIG. 3. Cells were treated with vehicle control or GZ17-6.02 (2 μM). Cells were fixed in place 6 hours after treatment and immunostaining of single cells performed to determine the levels of each protein/phosphor-protein (n=3+/−SD). * p<0.05 less than vehicle control; # p<0.05 greater than vehicle control.

Also, preclinical data demonstrates additional mechanisms which may explain 6.02's observed ability to enhance immunotherapy. In vitro assays designed to assess the viability and effects of GZ17-6.02, GZ17-6.02.02, its metabolites as single agents, and the metabolites in combination effects of CD8+ T cells and CD56+ natural killer cells after 72 hours of exposure. Test agents used included 6.02 (77% Isovanillin+13% Harmine+10% Curcumin), 6.02.02 (77% Isovanillin+13% Harmine+8.5% Curcumin sulfate tetrabutylammonium salt+1.5% Curcumin β-D-glucuronide), Curcumin metabolites combination (85% Curcumin sulfate tetrabutylammonium salt+15% Curcumin β-D-glucuronide), Curcumin metabolites single agents (single agent Curcumin sulfate tetrabutylammonium salt, or single agent Curcumin 3-D-glucuronide). Data demonstrated 6.02's ability to increase T cell and natural killer cell viability and activation at various concentrations, particularly at lower concentrations, as compared to other formulations. This data demonstrates additional mechanistic rational for 6.02's ability to further enhance immunotherapy.

TABLE
CD8 + T Cells
% of control

| Concen- | | | Curcumin | | |
|---|---|---|---|---|---|
| trations | | | Metabolites | Curcumin | Curcumin |
| (μM) | 6.02 | 602.02 | Combination | Sulfate | B-D-Glucuronide |
| 0.03 | 174.56 | 172.50 | 167.16 | 107.62 | 124.35 |
| 0.3 | 129.74 | 124.34 | 135.79 | 170.61 | 114.54 |
| 3 | 90.74 | 107.41 | 149.69 | 154.26 | 118.58 |
| 30 | 46.41 | 80.18 | 117.22 | 143.23 | 110.23 |

TABLE
CD56 + NK Cells
% of control

| Concen- | | | Curcumin | | |
|---|---|---|---|---|---|
| trations | | | Metabolites | Curcumin | Curcumin |
| (μM) | 6.02 | 602.02 | Combination | Sulfate | B-D-Glucuronide |
| 0.03 | 108.80 | 104.44 | 103.33 | 104.14 | 98.08 |
| 0.3 | 96.96 | 98.56 | 101.52 | 103.40 | 100.22 |
| 3 | 68.89 | 92.90 | 101.73 | 110.77 | 93.81 |
| 30 | 9.87 | 49.70 | 84.55 | 107.76 | 95.28 |

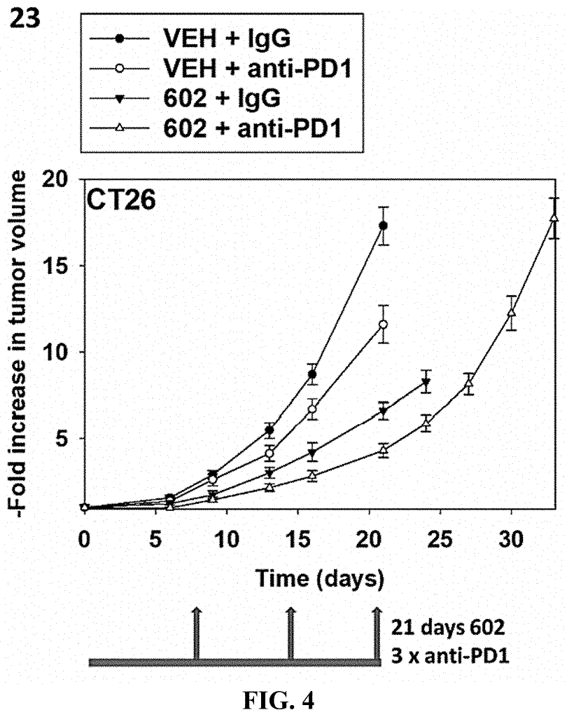

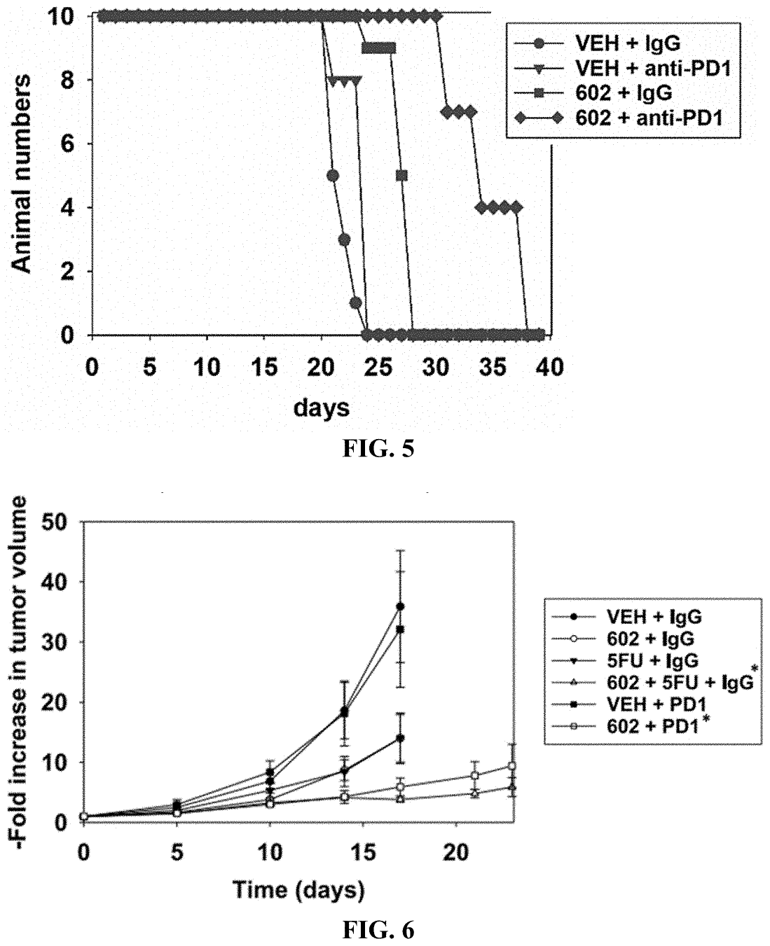

An initial animal study was performed to examine 602's ability to interact with anti-PD-1 immunotherapy to suppress tumor growth and prolong animal survival. Male immune-competent BALB/c mice (˜20 μg) were injected with 1.0×10⁶ male CT26 cells into their right rear flank (10 animals per treatment group). Tumors were permitted to form for 1 week with tumors at that time exhibiting a mean volume of approximately 65 mm³. Mice were treated by oral gavage once every day for 21 days with vehicle control or GZ17-6.02 (25 mg/kg). For antibody administrations, 7 days after the start of GZ-17-6.02 drug exposure animals are injected immunoprecipitation with a control IgG (25 μg) or an anti-PD-1 IgG (25 μg). Second (Day 14) and third (Day 21) antibody administrations were made. Before, during and after drug treatment tumors were measured using calipers, and tumor volume was assessed up to 38 days later. The -Fold increase in tumor volume under each condition is plotted is plotted in FIG. 4. Animals were humanely killed when the tumor volume reached approximately 2,000 mm³ due to ulceration, and the tumor and blood removed for further studies. Animal survival is plotted in FIG. 5 on a Kaplan Meier curve. (n=10+/−SD). #p<0.05 greater survival than vehicle+IgG animals; ##p<0.05 greater than 602+IgG animals; *p<0.05 reduced growth compared to 602+IgG animals.

A confirmatory animal study was then conducted using CT26 colorectal tumor cells that expressed a mutant K-RAS (Kirsten Rat Sarcoma) protein in syngeneic BALB/c (Bagg Albino) mice to determine whether GZ17-6.02 enhanced the efficacy of an anti-PD-1 antibody, and of 5 FU, in the mouse model. The mice were implanted with CT26 cells in their rear right flanks, and the resultant tumors were permitted to grow, with tumor volume measurement every 4-5 days, until tumor mean volumes were about 40 mm³. During the tumor growth period over 45 days, the mice were dosed daily with GZ17-6.02 (50 mg/kg) per os (p.o.), or weekly with 5 FU (25 mg/kg) p.o. A control antibody IgG (Immunoglobin G) or anti-PD-1 antibody was administered weekly at a level of 25 μg via intraperitoneal injection (i.p.).

The efficacy of the anti-PD-1 antibody and of 5 FU were both significantly enhanced by GZ17-6.02, as judged by measuring reduced tumor volumes over about 25 days, as shown in FIG. 6, and also depicted in the photographs in FIG. 7.

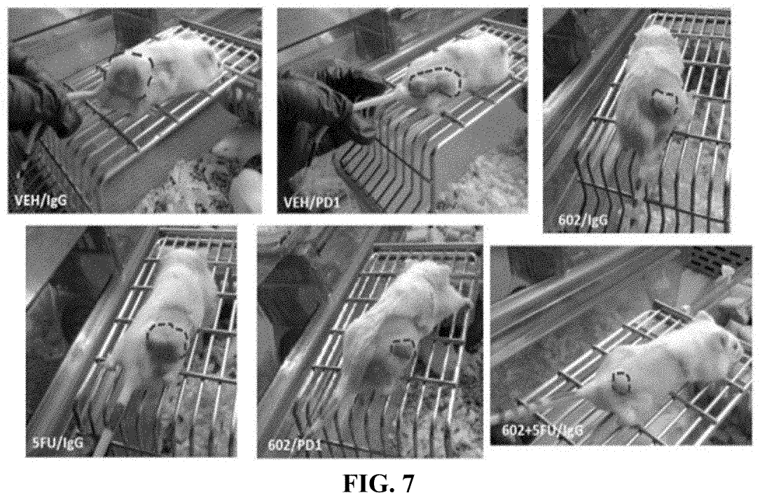

This study also confirmed enhanced animal survival past 45 days in those mice treated with GZ17-6.02 and either 5 FU or anti-PD-1 antibody. These treated mice had significantly greater survival rates as compared with those mice treated with GZ17-6.02 alone, with anti-PD-1 antibody alone, or with 5 FU alone, as shown in FIG. 8. FIG. 7 shows representative photographs of mouse tumors in each treatment group taken on Day 15. Three tumors from each treatment group were removed after death, digested, and the tumor cells isolated via culture in low serum media. Cells were plated and 24 h later fixed in place followed by in-cell western blotting performed against the indicated proteins and phospho-proteins as described in the Methods. The expression/phosphorylation of proteins in tumor cells derived from vehicle control tumors and from 602 treated tumors was determined (3 tumors in triplicate). Differences in protein expression/phosphorylation between 602-treated cells compared to vehicle control treated cells were determined (p<0.05) as shown below.

| Elevated | Reduced |
|---|---|
| P-AMPKα | FLIP-s |
| P-AKT T308 | CD95 |
| P-p70 S6K T389 | FAS-L |
| P-STAT5 Y694 | P-ULK1 S757 |
| Beclin1 | P-PERK |
| ATG5 | p65 NFκB |
| P-c-SRC Y416 | P-p65 NFκB |
| ERBB2 | CHOP |
| ERBB3 | P-JNK1/2 |
| P-ERBB1 | JAK2 |
| P-ERBB3 | P-JAK2 |
| P-PDGFRβ | c-SRC |
| GRP78 | P-c-SRC Y527 |
| ERK1/2 | c-KIT |
| MCL1 | |

### Example 2

To further understand the mechanistic interaction of GZ17-6.02 and 5-Fluorouracil leading to the unexpected and synergistic results observed in the CT26 mouse model, additional testing was carried out on combinations of these agents. These tests were performed in cell lines from various tissues to better understand 6.02 and 5FU's interactions in multiple tissue types. Early preclinical experiments lead researchers to examine various mechanisms surrounding endoplasmic reticulum (ER) stress signaling and autophagy as possible mechanisms of action leading to the greater than additive or synergistic effects observed in the CT26 mouse model.

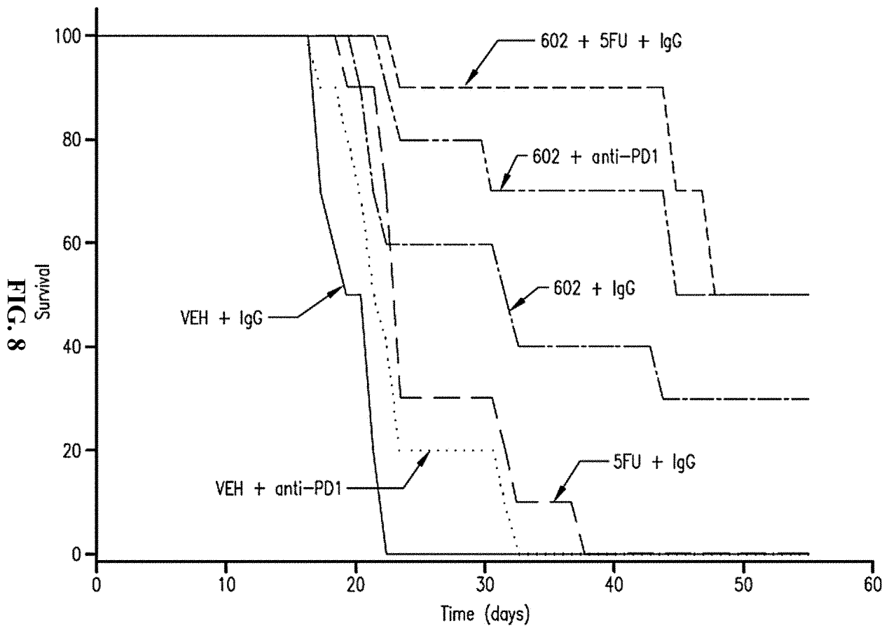

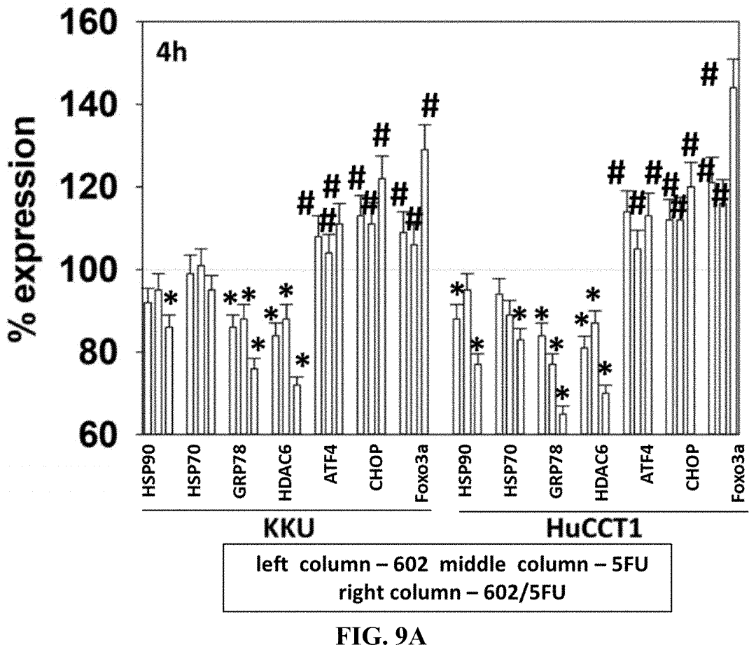

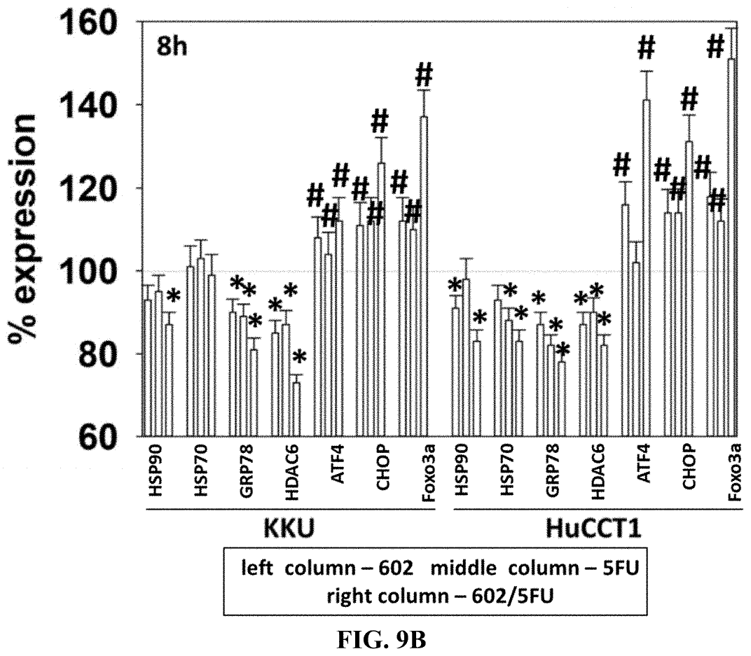

To examine the ER stress hypothesis, HuCCTl and KKU (cholangiocarcinoma) cells were plated in 96-well plates. Twenty-four hours later, cells were treated with vehicle control, 602, 5FU (50 μM) or the drugs in combination for 4 and 8 hours. Cells were fixed in situ, permeabilized, blocked, and stained overnight with the indicated validated primary antibodies. The following morning, the plates were washed, and secondary antibodies added carrying either 488 (green) or 594 nm (red) fluorescent tags. Images of the stained cells were brought into focus and then the Hermes WiScan internal software used to define the staining intensity of at least 120 cells per well/condition. The graphical data presented in FIGS. 9A and 9B are the normalized amount of fluorescence set at 100% comparing intensity values for vehicle control and 602 treatments (n=3+/−standard error) *p<0.05 less than vehicle; #p<0.05 greater than vehicle. These results demonstrate 6.02 and 5FU interact in a manner that increases ER stress-related proteins, such as ATF4, CHOP, and FOXO3A, while also causing reductions in chaperone and HDAC expression, further enhancing ER stress.

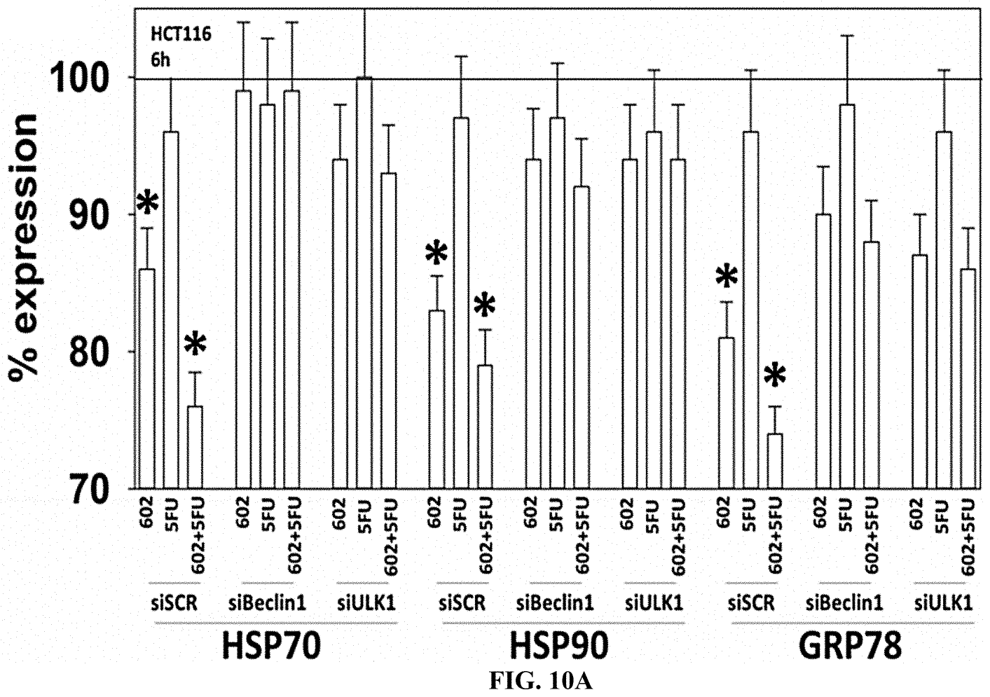

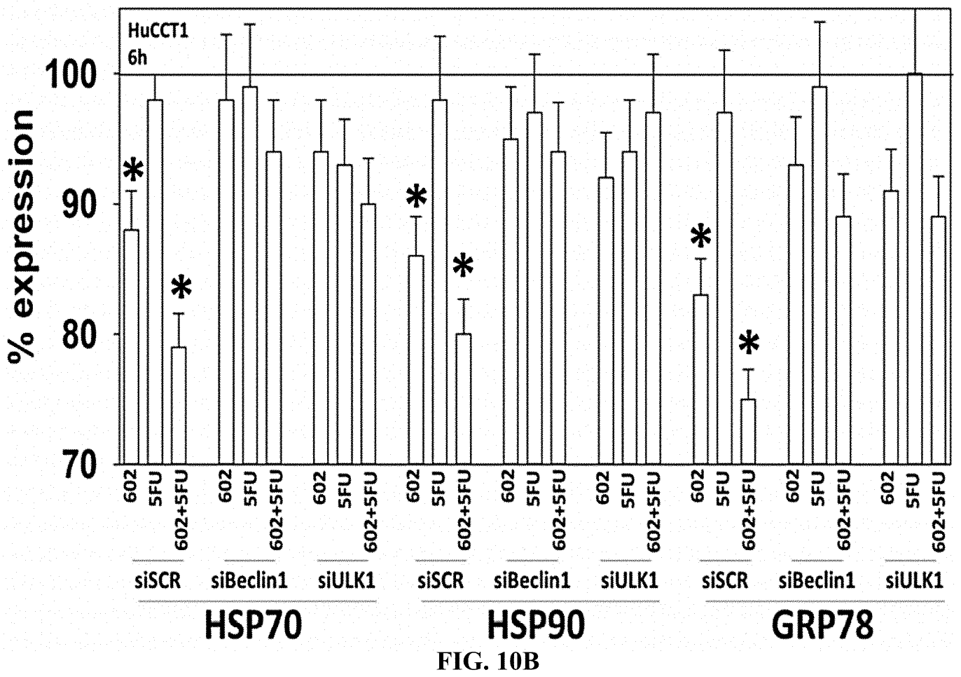

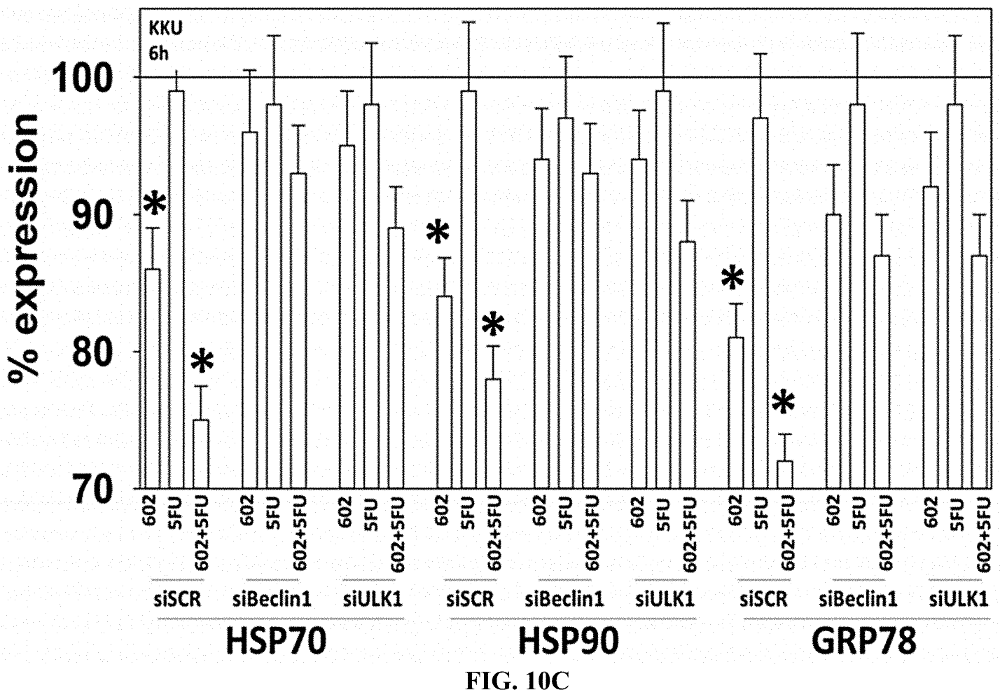

Further, chaperone expression decrease was observed in GI tumor cells. HCT116 cells were transfected with a scrambled siRNA control or with validated siRNA molecules to knock down the expression of the indicated proteins. 24 hours after transfection, cells were treated with vehicle control, 602 (2 μM), 5FU (50 μM) or the drugs in combination for 6 h. The expression of HSP70, HSP90 and GRP78 was assessed. Cells were fixed in situ, permeabilized, blocked and stained overnight with the indicated validated primary antibodies. The following morning, the plates were washed, and secondary antibodies added carrying either 488 nm (green) or 594 nm (red) fluorescent tags. Images of the stained cells were brought into focus and then the Hermes WiScan internal software used to define the staining intensity of at least 120 cells per well I condition. The graphical data presented in FIG. 10A, 10B, and 10C are the normalized amount of fluorescence set at 100% comparing intensity values for vehicle control and 602 treatments (n=3+/−SD). * p<0.05 less than corresponding values in siULK1 and siBeclin1. This data demonstrates the combination of 6.02 and 5FU induces a greater amount of ER stress compared to individual agents.

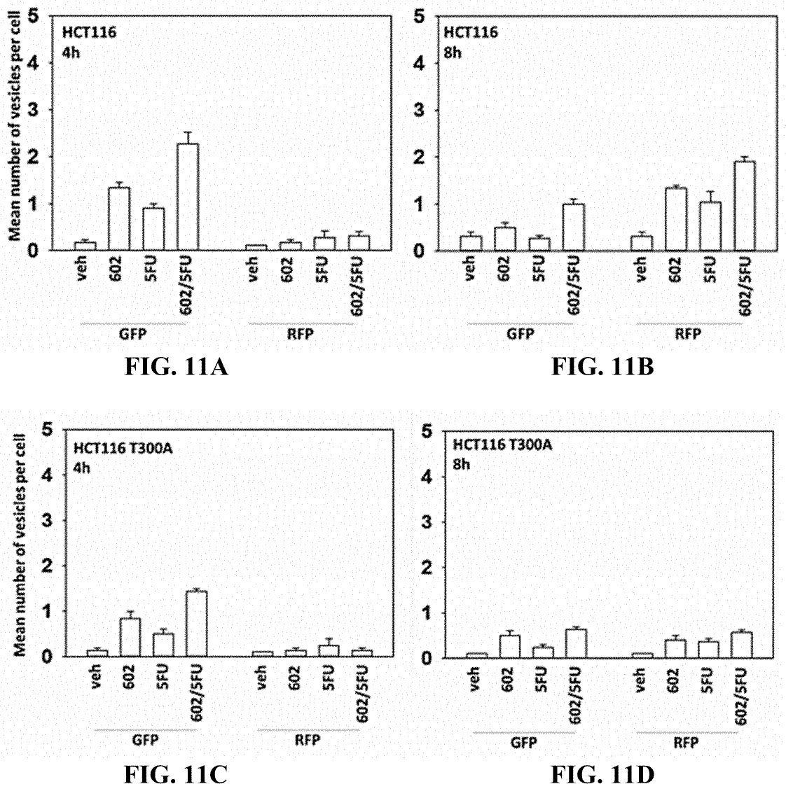

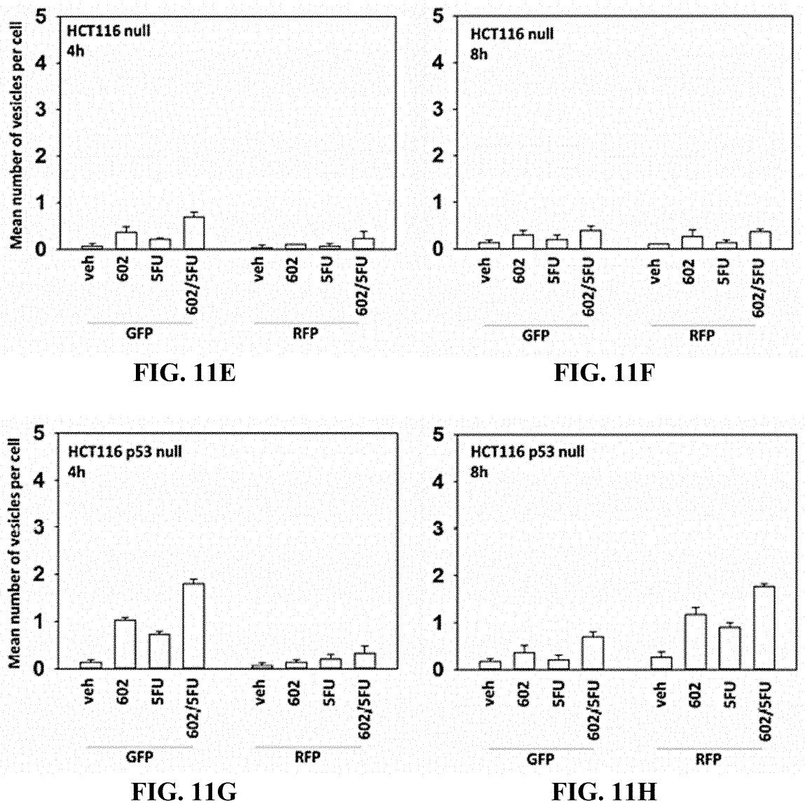

Autophagy was also implicated as a mechanism of action of the 6.02. Preclinical data suggested the combination of 6.02 and 5FU could enhance this process. HCT116 cells were transfected with a plasmid to express LC3-GFP-RFP. 24 hours after transfection, cells were treated with vehicle control, 602 (2 μM), 5FU (50 μM) or the drugs in combination for 4 h and for 8 h. At each time point the numbers of intense green GFP punctae and red RFP punctae were counted in over 40 cells per condition and the mean number of punctae per cell determined (n=3+/−SD). The results are shown in FIG. 11(A)-(H). This evidence of increased autophagic vesicle formation represents the combination's ability to spur on autophagy.

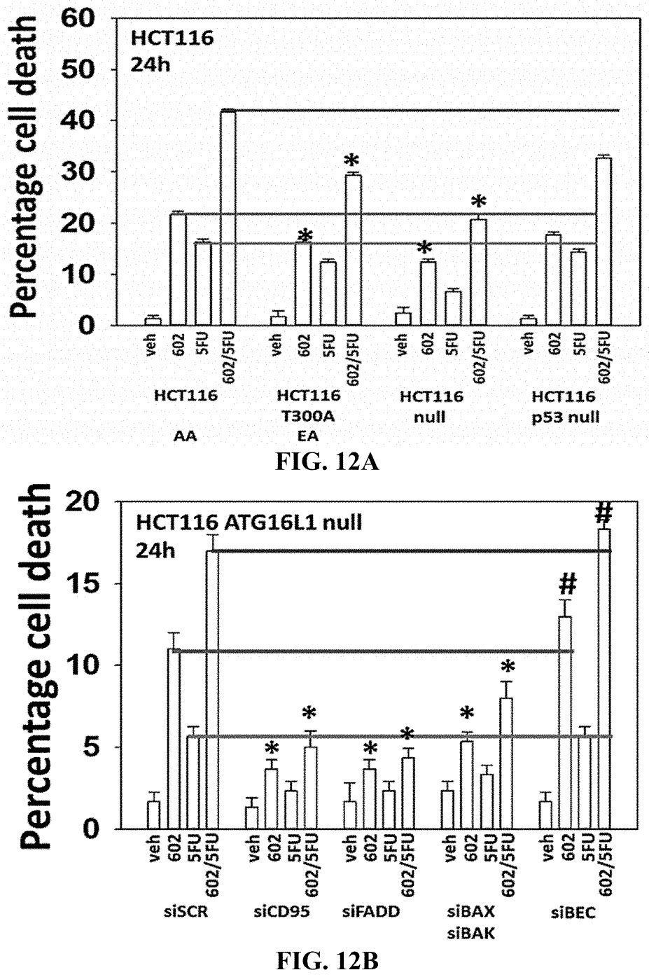

In subsequent experiments to determine lethality, HCT116 cells were treated with vehicle control 602 (2 μM), 5FU (50 μM) or the drugs in combination for 24 h. Cell viability was determined by trypan blue exclusion assays (n=3+/−SD), and the data is shown in the upper panel of FIG. 12A. In additional experiments, HCT116 cells, null for ATG16L1, were transfected with a scrambled siRNA or with validated siRNA molecules to knock down the expression of the indicated proteins. 24 hours after transfection, cells were treated with vehicle control, 602 (2 μM), 5FU (50 μM) or the drugs in combination for 24 h. Viability was determined by trypan blue exclusion assays (n=3+/−SD), and the results are shown in FIG. 12B. * p<0.05 less than corresponding value in wild type HCT116; # p<0.05 greater than corresponding value in siSCR control cells. This data indicates the combination of 6.02 and 5FU increase autophagic response resulting in increased cell death.

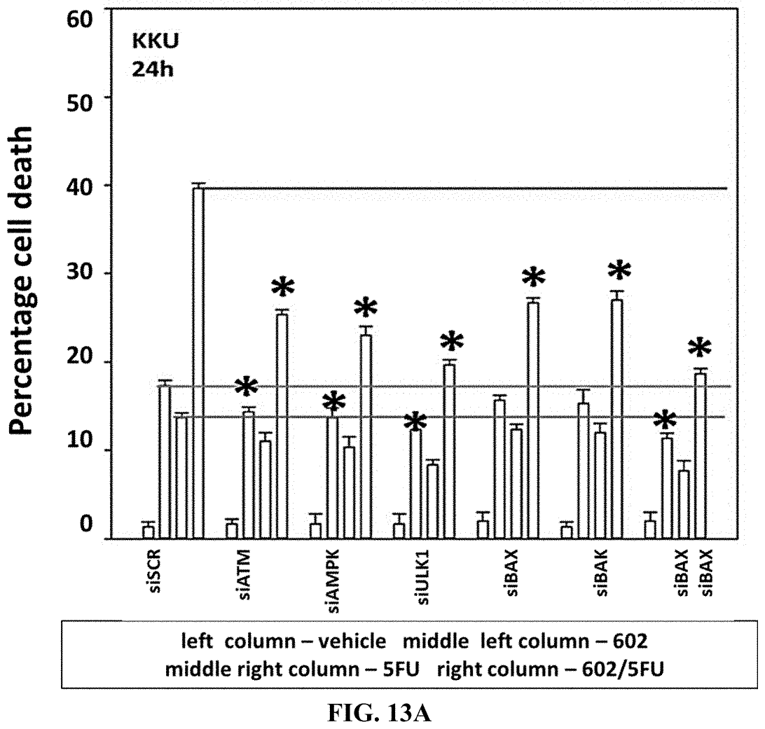

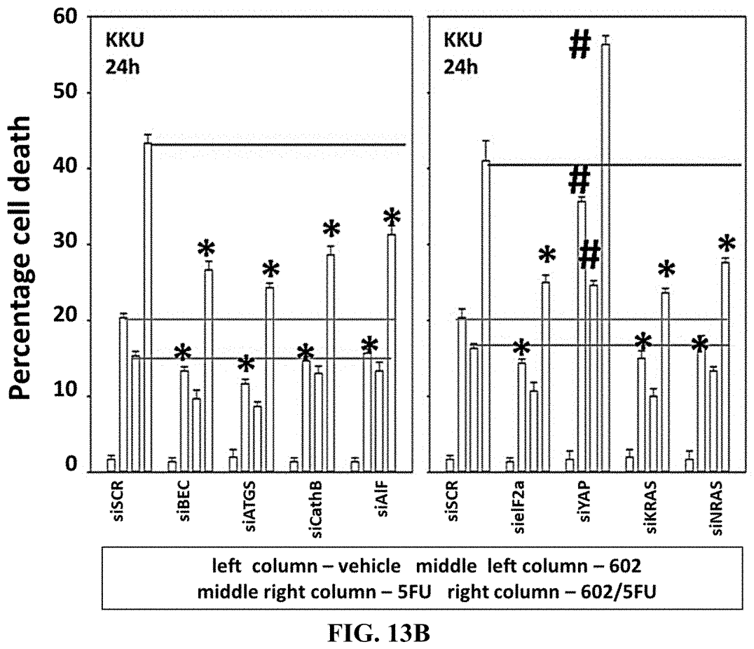

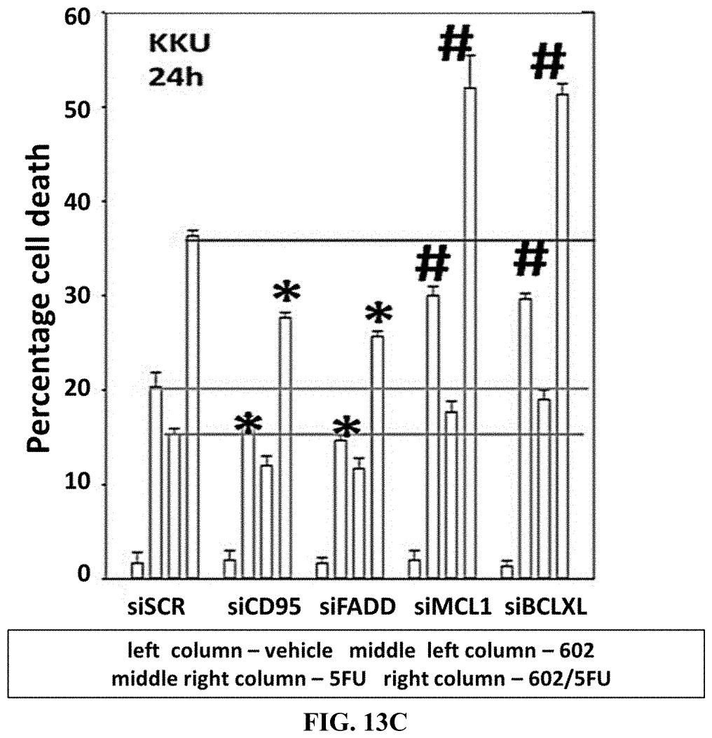

KKU (cholangiocarcinoma) cells were transfected with a scrambled control siRNA or were transfected with various confirmed siRNA molecules to knock down the expression of the indicated proteins. 24 hours after transfection cells were treated with vehicle control, 602 (2 μM), 5FU (50 μM) or the drugs in combination for 24 hours. Viability was determined by trypan blue exclusion assays (n=3+/−standard error), and the results are shown in FIGS. 13A, 13B, and 13C. *p<0.05 less than corresponding value in small interfering scrambled (siSCR); #p<0.05 greater than the corresponding value in siSCR.

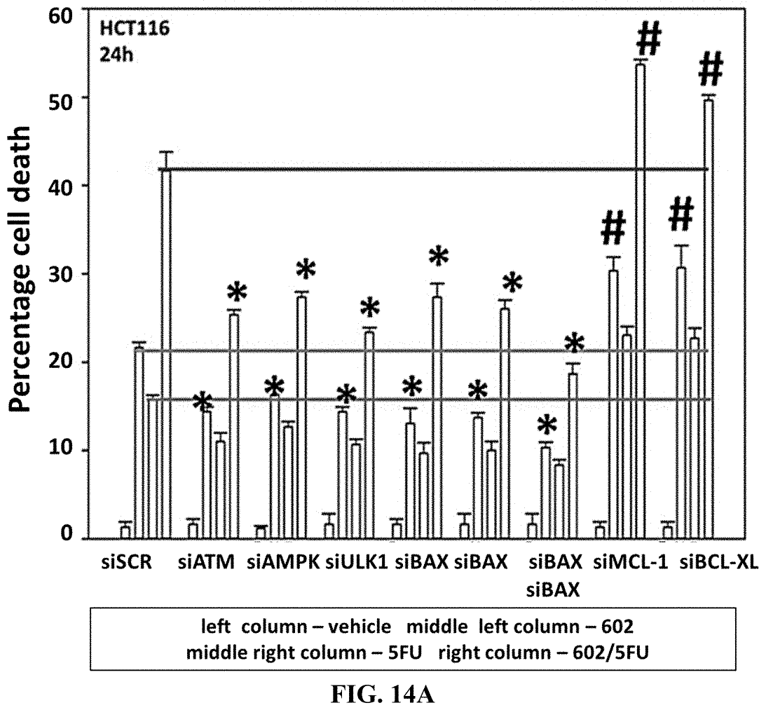

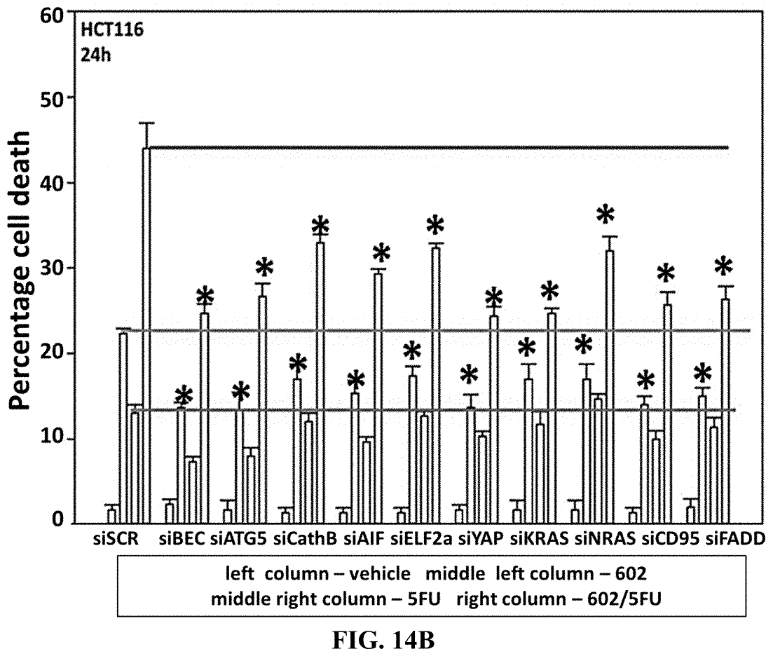

In additional experiments, HCT116 (colon) cells were transfected with a scrambled control siRNA or were transfected with various confirmed siRNA molecules to knock down the expression of the indicated proteins. Twenty-four hours after transfection cells were treated with vehicle control 602 (2 μM), 5FU (50 μM) or the drugs in combination for 24 hours. Viability was determined by trypan blue exclusion assays (n=3+/−standard error), and the data is shown in FIGS. 14A and 14B. *p<0.05 less than corresponding value in small interfering scrambled; #p<0.05 greater than the corresponding value in small interfering scrambled.

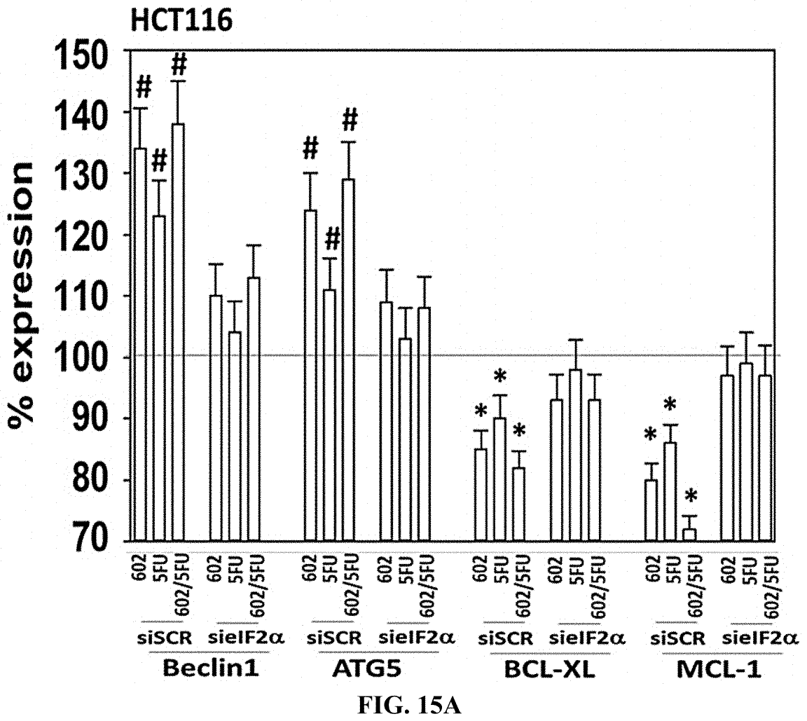

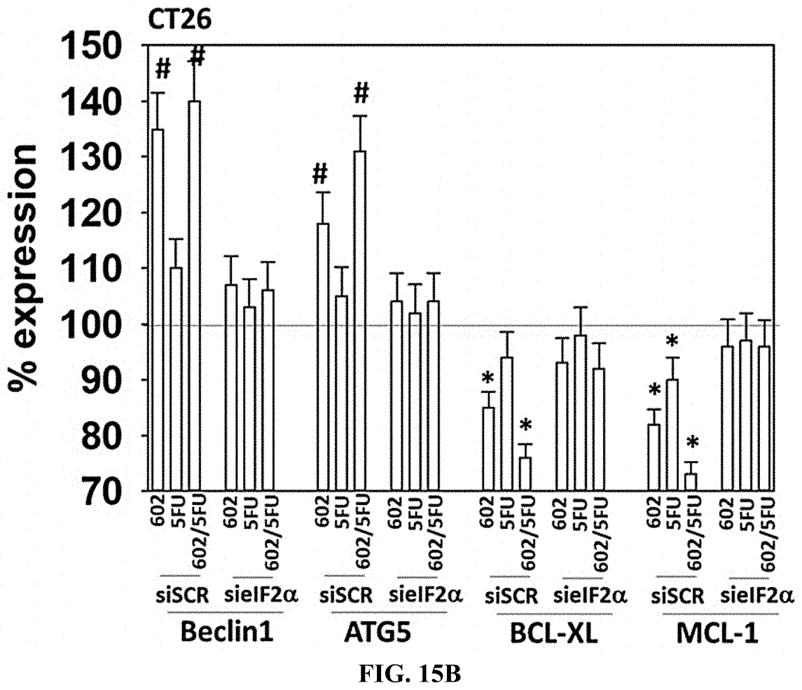

As shown in FIGS. 15A and 15B, knockdown of elF2-alpha alters the drug-induced expression alterations of Beclin1, ATG5, BCL-XL, and MCL-1. HCT116 and CT26 cells (colorectal) were transfected with a scrambled siRNA or with a validated siRNA to knock down the expression of elF2a. 24 hours after transfection cells were treated with vehicle control 602 (2 μM), 5FU (50 μM) or the drugs in combination for 6 hours. Cells were fixed in place, and immunostaining performed to detect the expression levels of Beclin1, ATG5, BCL-XL, and MCL-1 (n=3+/−standard error). *p<0.05 less than vehicle control; #p<0.05 greater than the corresponding value in sielF2-alpha cells.

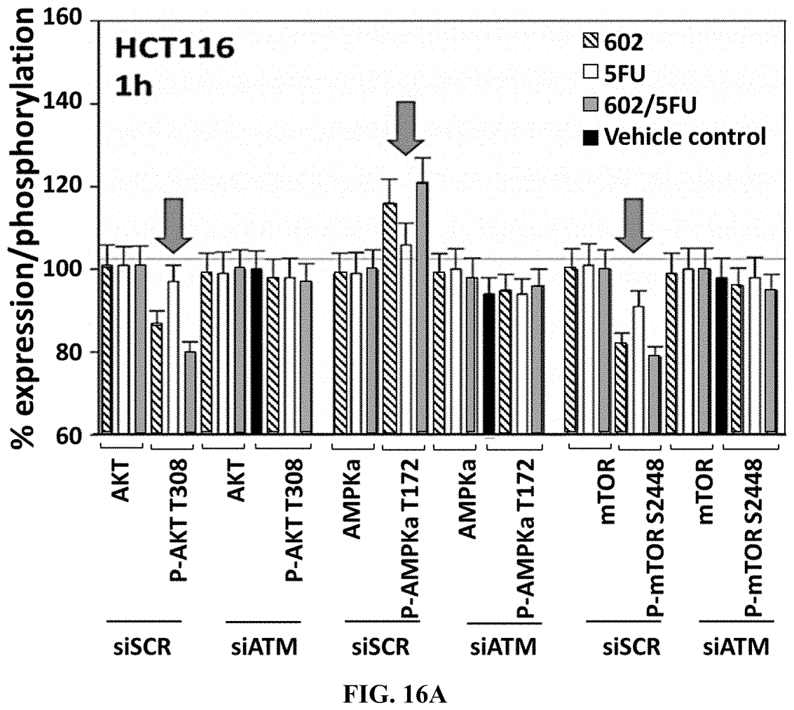

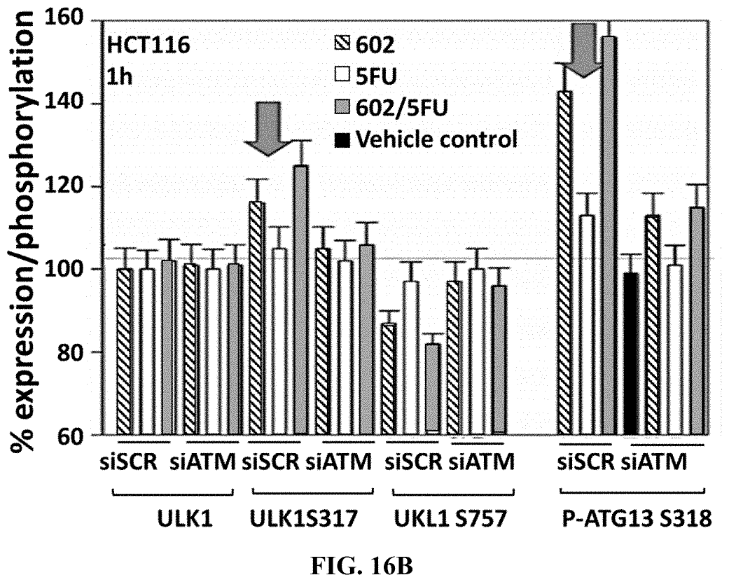

Ataxia-Telangiectasia Mutated (ATM) gene is a gene commonly implicated in autophagy. Preclinical data showed that the combination of 6.02 and 5FU acted to decrease ATM expression in a statistically significant manner. HCT116 colorectal cancer cells were transfected with a scrambled siRNA control or with a validated siRNA to knockdown ATM expression. 24 hours after transfection, cells were treated with vehicle control, 602 (2 μM), 5FU (50 μM) or the drugs in combination for 1 hour. Cells were fixed in situ, permeabilized, and immunostaining performed against the proteins shown in the graph (n=3+/−standard error) in FIGS. 16A & 16B. *p<0.05 less than corresponding vehicle control cells; #p<0.05 less than the corresponding value in small interfering scrambled cells. Brown arrows indicate where p<0.05 significant changes in protein phosphorylation occurred in an ATM-dependent fashion.

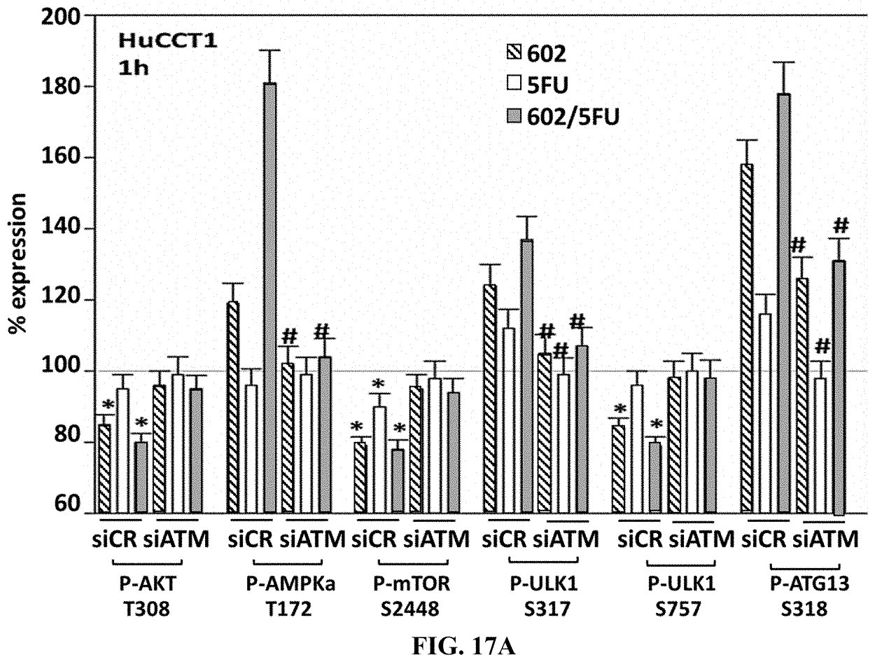

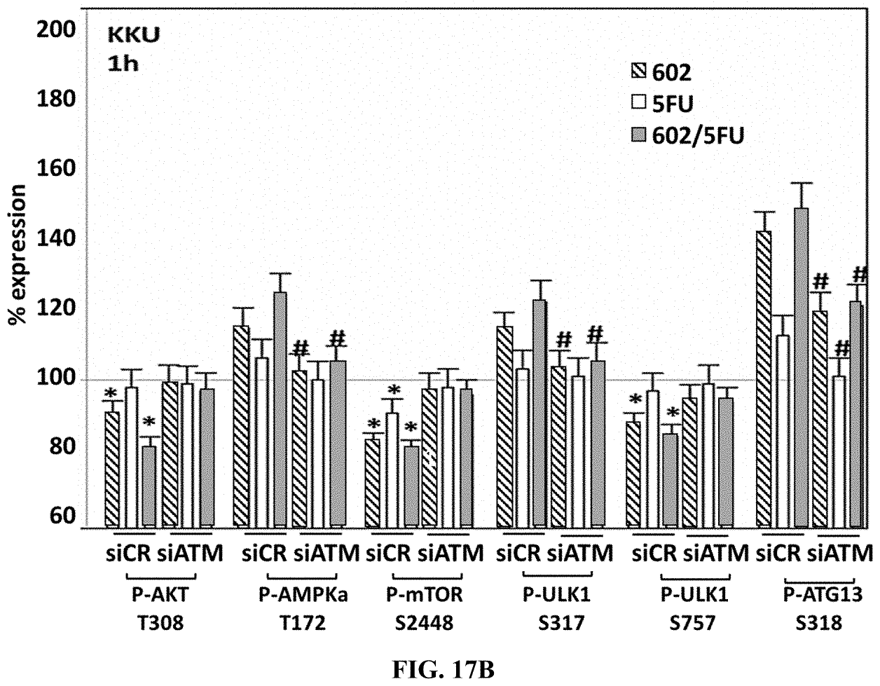

Consistent with the ATM related signaling data observed in the HCT116 cell line, changes in ATM phosphorylation were observed in HuCCT1 and KKU cell lines. These data indicate the activation of autophagy regulatory proteins via the activation of ATM. HuCCTl and KKU cholangiocarcinoma cells were transfected with a scrambled siRNA control or with a validated siRNA to knock down ATM expression. 24 hours after transfection, cells were treated with vehicle control, 602 (2 μM), 5FU (50 μM) or the drugs in combination for 1 h. Cells were fixed in situ, permeabilized and immunostaining performed against the proteins shown in the graph in FIGS. 17A and 17B (n=3+/−SD) * p<0.05 less than corresponding vehicle control cells; # p<0.05 less than corresponding value in siSCR cells.

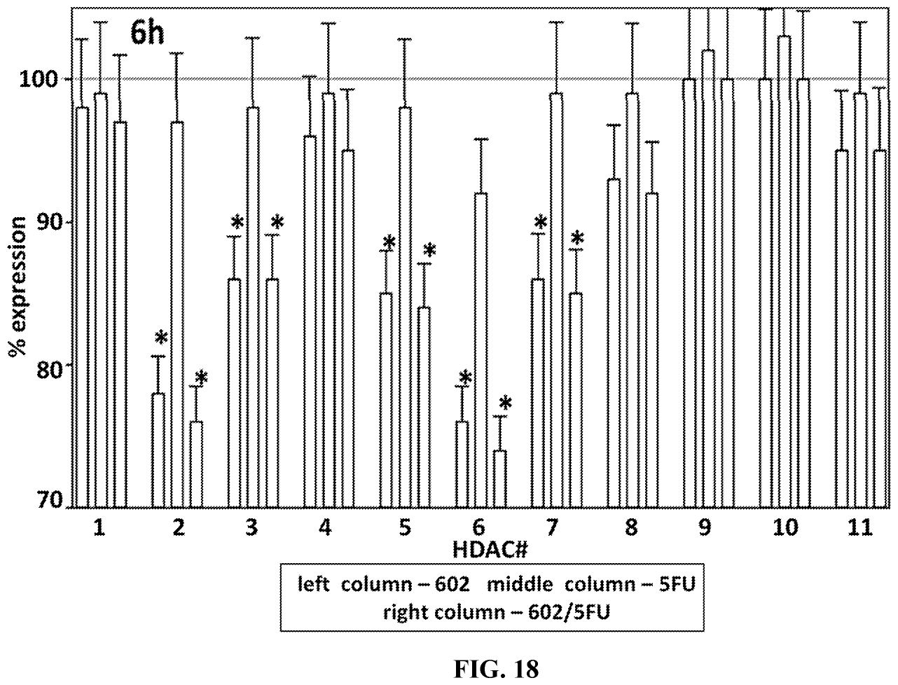

To determine additional processes affected by the combination of 6.02 and 5FU regarding cellular signaling and protein expression, Histone Deacetylase (HDAC) expression was examined. CT26 cells were treated with vehicle control or with 602 (final curcumin concentration 2.0 μM) in the presence or absence of 5FU (50 μM). Cells were fixed in situ 6 hours after drug exposure. Fixed cells were permeabilized, blocked, and stained overnight with the indicated validated primary antibodies. The following morning, the plates were washed, and secondary antibodies added carrying either 488 nm (green) or 594 nm (red) fluorescent tags. Images of the stained cells were brought into focus and then the Hermes WiScan internal software used to define via random selection the staining intensity of at least 120 cells per well/condition. The graphical data presented in FIG. 18 are the normalized amount of fluorescence set at 100% comparing intensity values for vehicle control and 602/5FU treatments (n=3+/−standard error). *p<0.05 less than vehicle control value. Decreases in expression were observed in multiple HDACs, including HDAC 2, 3, 5, 6, and 7.

### Example 4

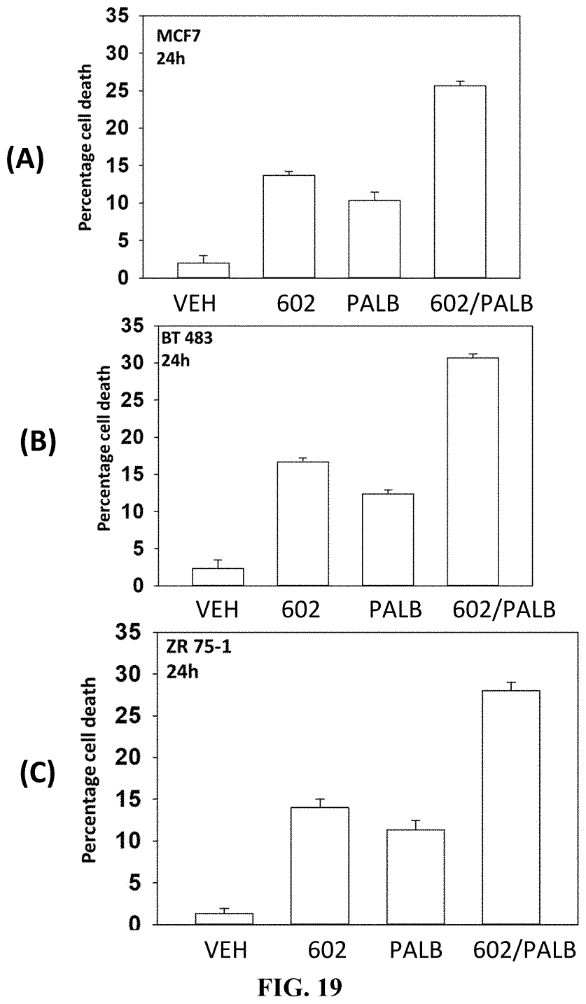

CDK 4/6 inhibitors, palbociclib, ribociclib, and abemaciclib, among others, have a unique mechanism of action and are currently a standard of care therapy in estrogen receptor positive, HER2 negative breast cancer. Data in multiple cell lines has shown GZ17-6.02 interacts with palbociclib to kill ER+ breast cancer cells, as shown in FIG. 19A-C.

In both ZR75 and MCF7 cells, palbociclib activates ERBB3, an effect that is blocked by GZ17-6.02. This data demonstrates 6.02 may have an ability to prevent the creation ofan escape pathway used by breast cancer to create resistance to CDK 4/6 inhibitor treatment. A similar effect was observed in one cell line for PDGFR beta. Although the amplitude of the effects on NEDD4 and PTEN were not significant, the trend agreed with the observation of reduced NEDD4 expression leading to/associated with increased PTEN expression, and inactivation of AKT which were observed.

TABLE 1

| | | | 6.02 + | | | | 6.02 + |
|---|---|---|---|---|---|---|---|
| ZR75 3 h | 6.02 | Palbo | Palbo | ZR75 6 h | 6.02 | Palbo | Palbo |
| ERBB3 | 100 | 100 | 100 | ERBB3 | 100 | 99 | 100 |
| P-ERBB3 | 100 | 115 | 100 | P-ERBB3 | 100 | 116 | 100 |
| PDGFR- | 101 | 100 | 100 | PDGFR- | 101 | 100 | 100 |
| Beta | | | | Beta | | | |
| P-PDGFR- | 95 | 100 | 93 | P-PDGFR- | 95 | 98 | 89 |
| Beta | | | | Beta | | | |
| NEDD4 | 99 | 100 | 98 | NEDD4 | 96 | 97 | 95 |
| PTEN | 100 | 101 | 100 | PTEN | 101 | 101 | 102 |

TABLE 2

| | | | 6.02 + | | | | 6.02 + |
|---|---|---|---|---|---|---|---|
| MCF7 3 h | 6.02 | Palbo | Palbo | MCF7 6 h | 6.02 | Palbo | Palbo |
| ERBB3 | 102 | 101 | 101 | ERBB3 | 100 | 100 | 100 |
| P-ERBB3 | 86 | 111 | 93 | P-ERBB3 | 90 | 114 | 86 |
| PDGFR- | 100 | 100 | 100 | PDGFR- | 99 | 99 | 100 |
| Beta | | | | Beta | | | |
| P-PDGFR- | 92 | 103 | 90 | P-PDGFR- | 84 | 105 | 92 |
| Beta | | | | Beta | | | |
| NEDD4 | 95 | 96 | 91 | NEDD4 | 94 | 97 | 92 |
| PTEN | 101 | 103 | 104 | PTEN | 103 | 103 | 106 |

With respect to cyclin D and cyclin E, a trend was observed. Palbociclib trended to increase the levels of both cyclins by 5-10%, an effect that was abolished by GZ17-6.02. This action is important to the efficacy of CDK 4/6 inhibitors as these drugs inhibit cyclin D from binding to CDK 4/6.

TABLE 3

| | | | 6.02 + | | | | 6.02 + |
|---|---|---|---|---|---|---|---|
| ZR75 3 h | 6.02 | Palbo | Palbo | ZR75 6 h | 6.02 | Palbo | Palbo |
| Cyclin D | 100 | 108 | 102 | Cyclin D | 100 | 107 | 101 |
| Cyclin E | 99 | 103 | 100 | Cyclin E | 98 | 105 | 97 |
| | | | 6.02 + | | | | 6.02 + |
| MCF7 3 h | 6.02 | Palbo | Palbo | MCF7 6 h | 6.02 | Palbo | Palbo |
| Cyclin D | 92 | 104 | 91 | Cyclin D | 98 | 108 | 101 |
| Cyclin E | 99 | 104 | 100 | Cyclin E | 98 | 104 | 100 |

ZR75-1 and MCF7 cells were treated with vehicle control, GZ17-6.02, Palbociclib or the drugs combined for 3 hours and for 6 hours. Cells were fixed in place and immuno-staining performed with in house validated antibodies to detect protein expression and protein phosphorylation. Although there is a great deal of data in later tables, the information can be clustered into specific groups of signaling proteins and signaling pathways. The drugs alone, and especially in combination, activate an ATM-AMPK-ULK1 S317 pathway which is causal at increasing ATG13 S318 phosphorylation, which subsequently triggers autophagosome formation. In these cell lines it was observed that basal activity of mTOR was low compared to tumor cells that express mutant RAS proteins. However, it was observed that the drug combination inactivated mTORC1 S2448 which is causal in reduced ULK1 S757 phosphorylation which promotes activation of ULK1.

Activation of endoplasmic reticulum (ER) stress signaling was observed by increased phosphorylation of PERK and its substrate eIF2alpha. Downstream of this as increased expression of GRP78 and CHOP was also observed. Also found were increased levels of Beclin1 and ATG5 which requires eIF2alpha phosphorylation.

GZ17-6.02 interacts with Palbociclib to activate autophagy, primarily via ATM and the AMPK. The activation of the AMPK was particularly robust. Increased activation phosphorylation of ULK1 S317 was noted whereas the relative inactivation of mTORC1 and mTORC2 and the reduction in ULK1 S757 phosphorylation also was observed. Additionally, ATG13 S318 phosphorylation, the activation of ULK1 contributed autophagy activation. (Palbociclib Tables 1 and 2)

The GZ17-6.02 Palbociclib combination caused a robust increase in the phosphorylation of PERK and eIF2 alpha and alongside this initial ER stress response were observed downstream outcomes of elevated expression of Beclin1, ATG5, GRP78 and CHOP. The Hippo pathway was inactivated as judged by elevated phosphorylation of YAP 5109, YAP S127, YAP S397 and TAZ S89. (Palbociclib Tables 1 and 2)

The levels of various HDACs were variably reduced by the drug 6.02 palbociclib combination based on the cell line, however expression of HDAC6 was robustly reduced in both cell lines. These changes in HIDAC protein expression show the ability of the 6.02 palbociclib combination to impact other signaling cascades. (Palbociclib Tables 1 and 2)

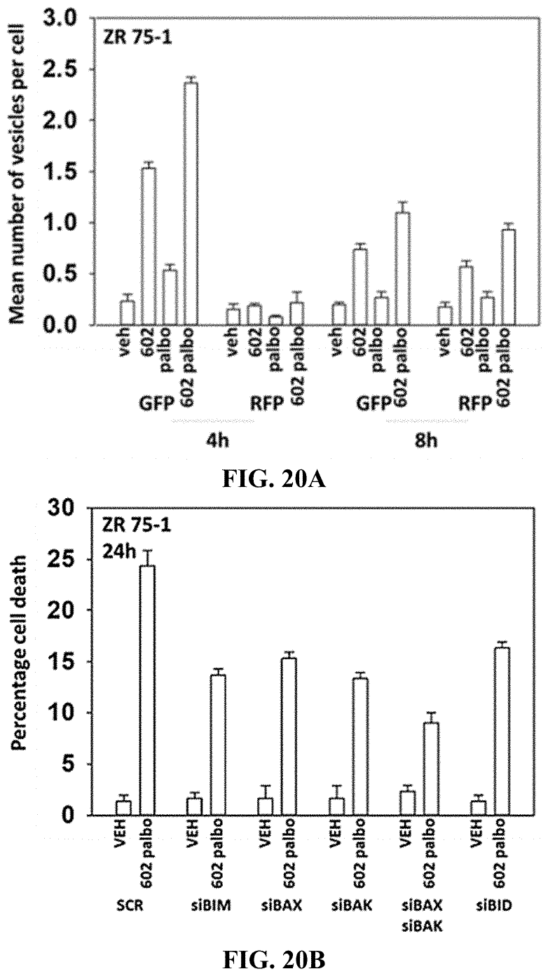

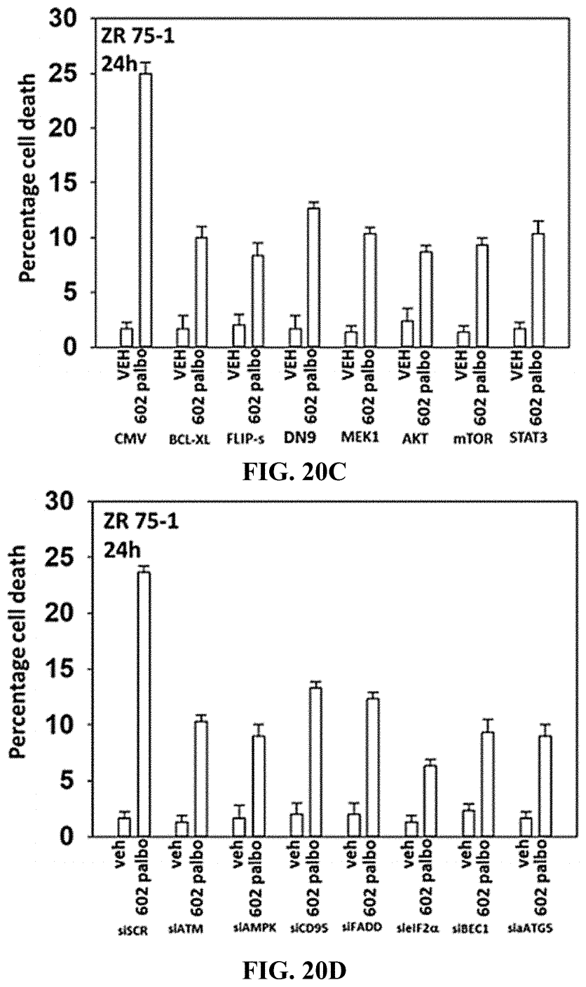

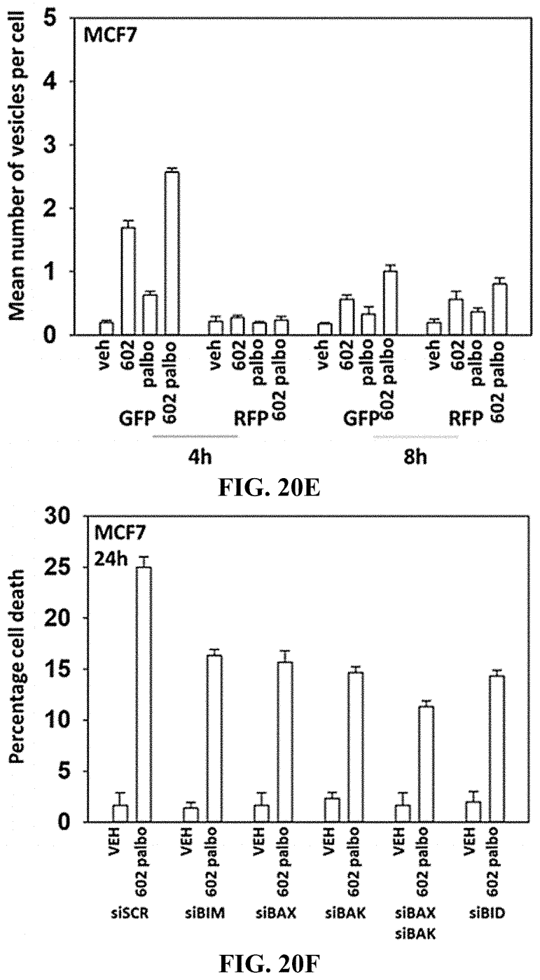

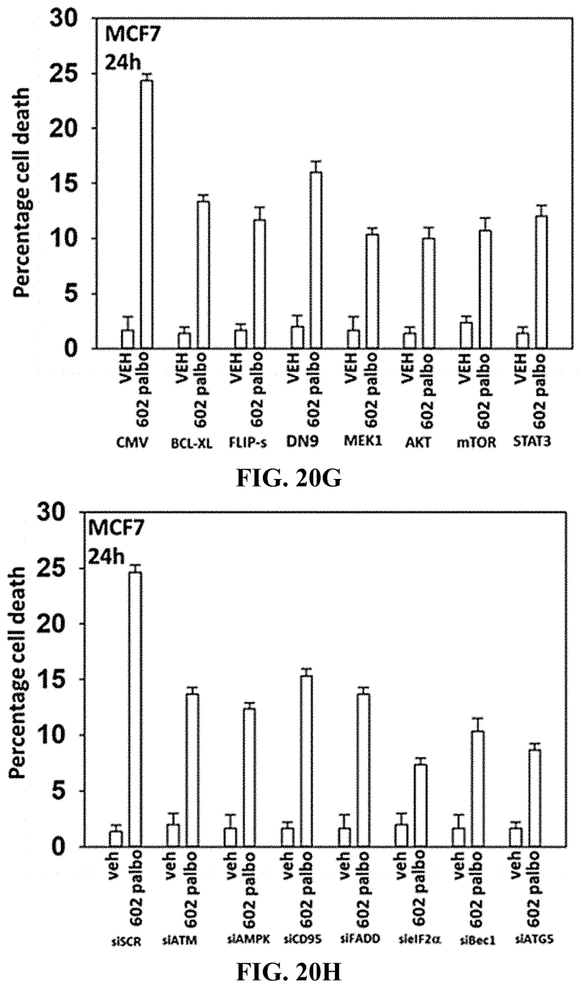

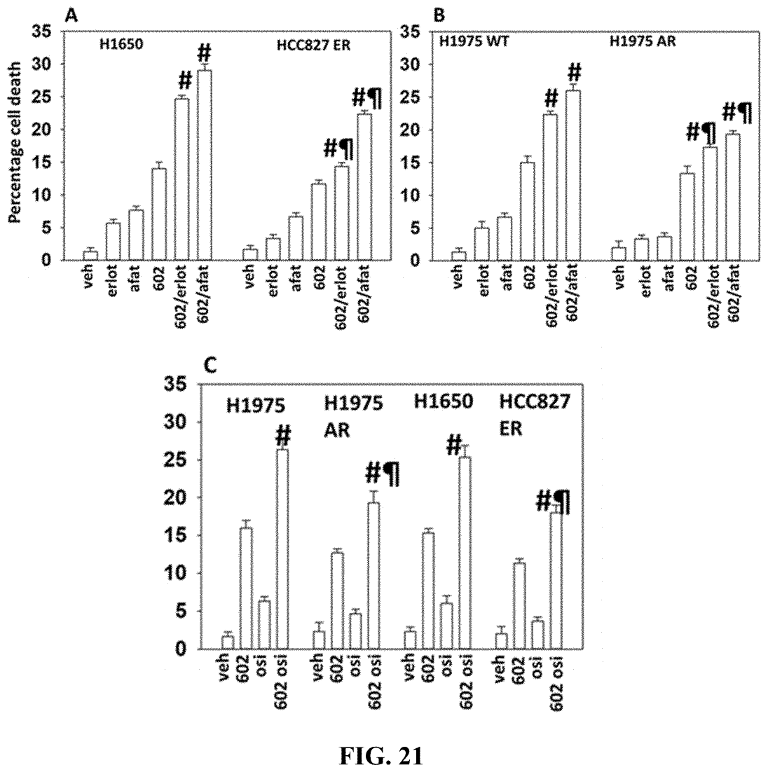

An impact of the combination on the regulation of pro- and anti-apoptotic proteins also was observed. Reduction in flcl-xl and MclI expression and the induction of Bax, Bak and Blim levels demonstrated these changes. Additionally, a drug-induced increase in FAS-ligand levels was also observed. (Palbociclib Tables 1 and 2) See also FIGS. 20A-20H.

PALBOCICLIB TABLE 1

| | ZR75, 3 h | | | ZR75, 6 h | | |
|---|---|---|---|---|---|---|
| | | | 602 + | | | 602 + |
| | 602 | PALBO | PALBO | 602 | PALBO | PALBO |
| ERBB1 | 99 | 99 | 97 | 100 | 100 | 100 |
| P-ERBB1 | 101 | 101 | 99 | 98 | 100 | 97 |
| ERBB2 | 99 | 99 | 99 | 100 | 100 | 101 |
| P-ERBB2 | 92 | 96 | 89 | 94 | 98 | 92 |
| ERBB3 | 100 | 100 | 100 | 100 | 99 | 100 |
| P-ERBB3 | 100 | 115# | 100 | 100 | 116# | 100 |
| ERRB4 | 100 | 100 | 100 | 99 | 99 | 99 |
| P-ERBB4 | 98 | 107 | 98 | 98 | 101 | 98 |
| PDGFRb | 101 | 100 | 100 | 101 | 100 | 100 |
| P-PDGFRb | 95 | 100 | 93 | 95 | 98 | 89 |
| ATM | 100 | 99 | 100 | 99 | 100 | 99 |
| P-ATM | 108 | 106 | 109 | 110 | 109 | 116# |
| AMPKa | 100 | 100 | 100 | 99 | 100 | 100 |
| P-AMPKa | 109 | 115# | 122# | 112 | 117# | 125# |
| mTOR | 100 | 101 | 101 | 99 | 100 | 100 |
| P-S2448 | 88 | 97 | 87* | 86* | 98 | 79* |
| P-S2481 | 89 | 96 | 88 | 87* | 98 | 86* |
| ULK1 | 101 | 100 | 100 | 99 | 100 | 100 |
| P-757 | 93 | 96 | 87* | 89 | 97 | 85* |
| P-317 | 107 | 112 | 115# | 112 | 114# | 117# |
| eIF2a | 100 | 99 | 99 | 101 | 100 | 99 |
| P-eIF2a | 114# | 110 | 114# | 115# | 109 | 116# |
| PERK | 100 | 101 | 100 | 101 | 99 | 102 |
| P-PERK | 111 | 111 | 114# | 113# | 111 | 116# |
| Beclin1 | 109 | 112 | 114# | 113# | 118# | 121# |
| ATG5 | 105 | 107 | 114# | 114# | 114# | 119# |
| ATG13 | 102 | 101 | 101 | 100 | 100 | 101 |
| P-ATG13 | 103 | 113# | 116# | 107 | 121# | 127# |
| GRP78 | 105 | 108 | 110 | 110 | 112 | 117# |
| CHOP | 106 | 107 | 112 | 105 | 109 | 112 |
| PP1 | 99 | 99 | 99 | 102 | 99 | 101 |
| NFkB | 100 | 100 | 100 | 101 | 101 | 100 |
| P-NFkB | 101 | 100 | 98 | 100 | 98 | 93 |
| c-SRC | 102 | 101 | 99 | 101 | 101 | 101 |
| Y416 | 100 | 101 | 100 | 97 | 102 | 96 |
| Y527 | 105 | 99 | 107 | 106 | 98 | 107 |
| c-MET | 100 | 100 | 99 | 101 | 101 | 100 |
| P-MET | 100 | 101 | 105 | 94 | 99 | 92 |
| CD95 | 100 | 100 | 100 | 100 | 101 | 101 |
| FAS-L | 103 | 108 | 110 | 104 | 109 | 113# |
| JAK2 | 100 | 100 | 100 | 101 | 100 | 99 |
| P-JAK2 | 97 | 100 | 97 | 97 | 99 | 97 |
| c-KIT | 100 | 100 | 100 | 100 | 100 | 100 |
| P-KIT | 94 | 100 | 94 | 95 | 100 | 95 |
| STAT3 | 101 | 100 | 100 | 100 | 99 | 98 |
| P-Y705 | 95 | 99 | 95 | 92 | 99 | 89 |
| STAT5 | 100 | 100 | 100 | 100 | 99 | 91 |
| P-Y694 | 95 | 100 | 95 | 93 | 99 | 91 |
| AKT | 101 | 100 | 100 | 100 | 101 | 100 |
| P-T308 | 91 | 106 | 89 | 90 | 106 | 85* |
| p70 S6K | 100 | 100 | 100 | 100 | 99 | 100 |
| P-p70 | 97 | 100 | 97 | 97 | 101 | 98 |
| T389 | | | | | | |
| ERK1/2 | 100 | 100 | 99 | 100 | 100 | 100 |
| P-ERK | 96 | 97 | 96 | 98 | 99 | 98 |
| p38 | 100 | 100 | 99 | 99 | 99 | 99 |
| P-p38 | 99 | 101 | 100 | 99 | 100 | 101 |
| JNK1/2 | 99 | 100 | 103 | 101 | 100 | 100 |
| P-JNK | 102 | 90 | 95 | 104 | 88 | 92 |
| LATS1/2 | 100 | 98 | 99 | 101 | 99 | 100 |
| P-1079 | 103 | 101 | 106 | 109 | 102 | 110 |
| P-909 | 107 | 98 | 108 | 111 | 109 | 112 |
| YAP | 98 | 98 | 99 | 98 | 99 | 99 |
| P-109 | 108 | 101 | 109 | 113# | 105 | 114# |
| P-127 | 114# | 100 | 122# | 117# | 106 | 119# |
| P-397 | 106 | 98 | 111 | 105 | 99 | 105 |
| TAZ | 100 | 100 | 100 | 101 | 102 | 101 |
| P-89 | 102 | 101 | 104 | 102 | 101 | 103 |
| ERK2 | 100 | 100 | 100 | 101 | 99 | 100 |
| HDAC1 | 90 | 101 | 90 | 89 | 102 | 86* |
| HDAC2 | 97 | 99 | 97 | 93 | 95 | 91 |
| HDAC3 | 88 | 96 | 85* | 87* | 97 | 86* |
| HDAC4 | 101 | 100 | 100 | 100 | 100 | 100 |
| HDAC5 | 100 | 98 | 100 | 100 | 101 | 101 |
| HDAC6 | 80* | 99 | 75* | 77* | 98 | 75* |
| HDAC7 | 99 | 101 | 98 | 99 | 102 | 99 |
| HDAC8 | 101 | 100 | 100 | 101 | 99 | 100 |
| HDAC9 | 101 | 101 | 101 | 100 | 101 | 101 |
| HDAC10 | 98 | 98 | 88 | 100 | 100 | 93 |
| HDAC11 | 96 | 96 | 95 | 98 | 100 | 98 |
| BCL-XL | 92 | 94 | 88 | 93 | 91 | 89 |
| MCL1 | 97 | 96 | 85* | 95 | 91 | 84* |
| BAX | 101 | 106 | 107 | 103 | 108 | 110 |
| BAK | 106 | 107 | 108 | 108 | 109 | 111 |
| BIM | 106 | 109 | 111 | 111 | 113# | 115# |
| NEDD4 | 99 | 100 | 98 | 96 | 97 | 95 |
| PTEN | 100 | 101 | 100 | 101 | 101 | 102 |
| ER | 94 | 100 | 93 | 93 | 99 | 87* |
| PD-L1 | 90 | 100 | 89 | 87* | 99 | 85* |
| PD-L2 | 101 | 100 | 100 | 101 | 104 | 101 |
| MHCA | 106 | 102 | 106 | 107 | 103 | 112 |
| ODC | 97 | 100 | 97 | 101 | 98 | 103 |
| IDO1 | 97 | 96 | 96 | 100 | 100 | 102 |
| Cyclin D | 100 | 108 | 102 | 100 | 107 | 101 |
| Cyclin E | 99 | 103 | 100 | 98 | 105 | 97 |

PALBOCICLIB TABLE 2

| | MCF7, 3 h | | | MCF7, 6 h | | |
|---|---|---|---|---|---|---|
| | | | 602 + | | | 602 + |
| | 602 | PALBO | PALBO | 602 | PALBO | PALBO |
| ERBB1 | 99 | 99 | 98 | 100 | 101 | 101 |
| P-ERBB1 | 90 | 100 | 88 | 79* | 100 | 71* |
| ERBB2 | 99 | 100 | 99 | 99 | 100 | 100 |
| P-ERBB2 | 100 | 99 | 99 | 99 | 100 | 101 |
| ERBB3 | 102 | 101 | 101 | 100 | 100 | 100 |
| P-ERBB3 | 86* | 111 | 93 [symbol] | 90 | 114# | 86# [symbol] |
| ERRB4 | 99 | 99 | 100 | 101 | 100 | 100 |
| P-ERBB4 | 100 | 102 | 97 | 98 | 106 | 98 |
| PDGFRb | 100 | 100 | 100 | 99 | 99 | 100 |
| P- | 92 | 103 | 90 [symbol] | 84* | 105 | 92 [symbol] |
| PDGFRb | | | | | | |
| ATM | 100 | 100 | 100 | 100 | 99 | 100 |
| P-ATM | 108 | 105 | 110 | 112 | 109 | 115# |
| AMPKa | 100 | 102 | 99 | 100 | 100 | 100 |
| P-AMPKa | 110 | 112 | 114# | 112 | 117# | 118# |
| mTOR | 100 | 100 | 100 | 100 | 100 | 100 |
| P-S2448 | 89 | 98 | 86* | 89 | 98 | 86* |
| P-S2481 | 91 | 98 | 88 | 89 | 96 | 88 |
| ULK1 | 100 | 100 | 101 | 100 | 100 | 100 |
| P-757 | 94 | 98 | 93 | 92 | 98 | 89 |
| P-317 | 105 | 109 | 114# | 112 | 113# | 116# |
| eIF2a | 100 | 99 | 99 | 100 | 100 | 100 |
| P-eIF2a | 109 | 107 | 112 | 113# | 110 | 115# |
| PERK | 100 | 101 | 100 | 101 | 101 | 101 |
| P-PERK | 106 | 109 | 111 | 112 | 103 | 114# |
| Beclin1 | 110 | 113# | 117# | 112 | 117# | 120# |
| ATG5 | 109 | 107 | 112 | 117# | 114# | 117# |
| ATG13 | 101 | 100 | 101 | 102 | 101 | 102 |
| P-ATG13 | 106 | 116# | 120# | 108 | 118# | 127# |
| GRP78 | 112 | 114# | 119# | 117# | 115# | 117# |
| CHOP | 106 | 112 | 116# | 113# | 116# | 120# |
| PP1 | 107 | 98 | 108 | 109 | 99 | 109 |
| NFkB | 99 | 101 | 100 | 101 | 100 | 100 |
| P-NFkB | 97 | 100 | 89 | 88 | 99 | 87* |
| c-SRC | 100 | 100 | 101 | 100 | 101 | 100 |
| Y416 | 96 | 100 | 96 | 96 | 101 | 95 |
| Y527 | 113# | 98 | 114# | 109 | 103 | 110 |
| c-MET | 91 | 91 | 91 | 99 | 99 | 98 |
| P-MET | 92 | 100 | 93 | 98 | 96 | 90 |
| CD95 | 100 | 101 | 101 | 100 | 100 | 100 |
| FAS-L | 102 | 108 | 113# | 103 | 111 | 114# |
| JAK2 | 98 | 99 | 97 | 100 | 100 | 100 |
| P-JAK2 | 96 | 101 | 89 | 95 | 100 | 88 |
| c-KIT | 102 | 100 | 102 | 101 | 102 | 100 |
| P-KIT | 94 | 100 | 95 | 93 | 100 | 91 |
| STAT3 | 99 | 100 | 100 | 99 | 100 | 99 |
| P-Y705 | 98 | 98 | 93 | 91 | 98 | 87* |
| STAT5 | 100 | 100 | 100 | 100 | 100 | 100 |
| P-Y694 | 94 | 98 | 93 | 94 | 98 | 89 |
| AKT | 100 | 100 | 100 | 100 | 100 | 100 |
| P-T308 | 92 | 96 | 82* | 89 | 107 | 84* |
| p70 S6K | 99 | 99 | 99 | 100 | 100 | 100 |
| P-p70 | 97 | 105 | 100 | 85* | 110 | 95 [symbol] |
| T389 | | | | | | |
| ERK1/2 | 100 | 100 | 100 | 100 | 100 | 100 |
| P-ERK | 92 | 101 | 87* | 89 | 99 | 77* |
| p38 | 100 | 101 | 100 | 102 | 103 | 100 |
| P-p38 | 98 | 97 | 99 | 98 | 100 | 100 |
| JNK1/2 | 101 | 100 | 101 | 101 | 100 | 100 |
| P-JNK | 96 | 89 | 86* | 99 | 90 | 84* |
| LATS1/2 | 100 | 100 | 100 | 99 | 100 | 99 |
| P-1079 | 110 | 109 | 110 | 113# | 112 | 114# |
| P-909 | 107 | 99 | 102 | 113# | 99 | 105 |
| YAP | 100 | 99 | 100 | 99 | 99 | 99 |
| P-109 | 102 | 106 | 108 | 113# | 104 | 112 |
| P-127 | 109 | 102 | 111 | 115# | 104 | 115# |
| P-397 | 107 | 96 | 102 | 113# | 99 | 114# |
| TAZ | 101 | 101 | 100 | 102 | 100 | 101 |
| P-89 | 110 | 105 | 113# | 111 | 103 | 114# |
| ERK2 | 100 | 100 | 100 | 100 | 100 | 100 |
| HDAC1 | 97 | 100 | 95 | 86* | 99 | 80* |
| HDAC2 | 89 | 94 | 87* | 88 | 95 | 86* |
| HDAC3 | 94 | 97 | 87* | 99 | 101 | 90 |
| HDAC4 | 99 | 99 | 101 | 100 | 102 | 101 |
| HDAC5 | 100 | 99 | 99 | 100 | 98 | 100 |
| HDAC6 | 86* | 100 | 82* | 80* | 101 | 76* |
| HDAC7 | 99 | 100 | 98 | 97 | 101 | 97 |
| HDAC8 | 100 | 100 | 100 | 101 | 101 | 102 |
| HDAC9 | 99 | 99 | 100 | 101 | 100 | 102 |
| HDAC10 | 99 | 99 | 100 | 100 | 99 | 98 |
| HDAC11 | 100 | 100 | 100 | 99 | 99 | 101 |
| BCL-XL | 96 | 99 | 96 | 90 | 94 | 87* |
| MCL1 | 93 | 95 | 91 | 87* | 91 | 87* |
| BAX | 108 | 103 | 110 | 107 | 95 | 113# |
| BAK | 107 | 95 | 112 | 108 | 109 | 112 |
| BIM | 106 | 109 | 110 | 108 | 113# | 118# |
| NEDD4 | 95 | 96 | 91 | 94 | 97 | 92 |
| PTEN | 101 | 103 | 104 | 103 | 103 | 106 |
| ER | 96 | 99 | 96 | 88 | 91 | 85* |
| PD-L1 | 93 | 100 | 90 | 93 | 98 | 90 |
| PD-L2 | 99 | 100 | 97 | 98 | 98 | 98 |
| MHCA | 110 | 102 | 114# | 110 | 103 | 114# |
| ODC | 98 | 103 | 101 | 97 | 101 | 96 |
| IDO1 | 98 | 97 | 97 | 97 | 96 | 96 |
| Cyclin D | 92 | 104 | 91 [symbol] | 98 | 108 | 101 |
| Cyclin E | 99 | 104 | 100 | 98 | 104 | 100 |

In summary, alterations in the activity and function of growth factor receptors and their downstream intracellular signaling pathways were observed. Notably, palbociclib activated ERBB3, an effect blocked by GZ17-6.02. Other receptors such as the PDGFR beta trended towards being activated, effects which were also blocked by GZ17-6.02. The drug combination reduced the activities of AKT, ERK1/2 and JNK1/2, all of which imply reduced growth and increased cell death. Treatment of cells with palbociclib caused a non-significant trend to increase the expression of cyclin D1 and cyclin E, whereas GZ17-6.02 caused a non-significant trend to reduce the expression of cyclin D1 and cyclin E. The drug combination reduced cyclin D1 and cyclin E levels to those of GZ17-6.02 alone, which was a significant decrease below the palbociclib alone value. These events were also associated with increased expression of the death receptor ligand FAS-L and decreased expression of the protective proteins MCL1 and BCL-XL, and of the estrogen receptor itself. The expression of toxic BH3 domain proteins such as BAX, BAK and BIM were elevated. The Hippo pathway has been proposed as a mechanism by which stressed ER+ breast cancer cells can survive chemotherapy. GZ17-6.02, alone or combined with palbociclib, increased the phosphorylation of LATS1/2, YAP and TAZ, i.e., inactivation of the Hippo pathway.

Several rapid compensatory survival responses observed 3 hours to 6 hours after palbociclib exposure were mitigated by the addition of 6.02. Increased ERBB3 phosphorylation was blocked by GZ17-6.02. Downstream, this could be linked to altered phosphorylation of AKT. The levels of cyclins D and E trended upwards in response to palbociclib, and this trend was blocked by GZ17-6.02. And the drug combination inactivated YAP and TAZ. Hence three prior acknowledged palbociclib resistance mechanisms, elevated PI3K signaling, enhanced expression of cyclins D and E and altered YAP and TAZ phosphorylation have been observed in our data sets. GZ17-6.02 acted to suppress the induction of those resistance ERBB3 and cyclin mechanisms and caused inactivation of YAP/TAZ.

### Example 5

### GZ17-6.02 and Pemetrexed Interact to Kill Osimertinib-Resistant NSCLC Cells that Express Mutant ERBB1 Proteins

As demonstrated in this Example, we determined the molecular mechanisms by which the novel therapeutic GZ17-6.02 killed NSCLC cells. Erlotinib, afatinib and osimertinib interacted with GZ17-6.02 to kill NSCLC cells expressing mutant EGFR proteins. GZ17-6.02 did not interact with any EGFR inhibitor to kill osimertinib resistant cells. GZ17-6.02 interacted with the thymidylate synthase inhibitor pemetrexed to kill NSCLC cells expressing mutant ERBB1 proteins or mutant RAS proteins or cells that were resistant to EGFR inhibitors. The drugs interacted to activate ATM, the AMPK and ULK1 and inactivate mTORC1, mTORC2, ERK1/2, AKT, eIF2a and c-SRC. Knock down of ATM or AMPKa₁ prevented ULK1 activation. The drugs interacted to cause autophagosome formation followed by flux, which was significantly reduced by knock down of ATM, AMPKa₁, eIF2a, or by expression of an activated mTOR protein. Knock down of Beclin1, ATG5 or [BAX+BAK] partially though significantly reduced drug combination lethality as did expression of activated mTOR/AKT/MEK1 or over-expression of BCL-XL. Expression of dominant negative caspase 9 weakly reduced killing. The drug combination reduced the expression of HDAC2 and HDAC3 which correlated with lower PD-L1, IDO1 and ODC levels and increased MHCA expression. Collectively, our data support consideration of combining GZ17-6.02 and pemetrexed in osimertinib resistant NSCLC.

### Introduction

The drug GZ17-6.02 undergoing phase I evaluation in solid tumor patients (NCT03775525). GZ17-6.02 has three components that are natural chemicals; curcumin (10%); isovanillin (77%); and harmine (13%). The most widely studied compound is curcumin, i.e., turmeric, the spice most associated with Indian cuisine which is comprised ˜95% of curcumin and curcuminoid derivatives. The safe maximal plasma concentration of commercially available lecithin liposomal curcumin, e.g., MERIVA®, for an 800 mg ingestion is approximately 2 μM. Our prior in vitro studies have used GZ17-6.02 with the basal concentration of curcumin set at 2.0 μM. The plants Arum palaestinum and Peganum harmala been used for centuries in the Levant and Orient for the treatment of many ailments, including cancer. The most bio-active chemical isolated from these plants is harmine. Studies have shown that whilst harmine has anti-proliferative effects in tumor cells, the compound appears to lack any anti-proliferative biologic effects in non-transformed cells. We have previously shown that GZ17-6.02 interacted with 5-fluorouracil (5FU) to kill GI tumor cells, with doxorubicin to kill sarcoma cells and with [trametinib+dabrafenib] to kill cutaneous melanoma cells expressing B-RAF V600E. Our new studies were performed to determine whether GZ17-6.02 could kill non-small cell lung cancer (NSCLC) cells expressing mutant activated forms of the EGF receptor (ERBB1).

The treatment of NSCLC over the past 20 years has been revolutionized, first by the development of the pemetrexed carboplatin drug combination and then subsequently by checkpoint inhibitory immunotherapy. For NSCLC tumors expressing mutant RAS proteins or without a clear oncogenic driver, the combination of pemetrexed, carboplatin and an anti-PD1 antibody, e.g., pembrolizumab, is a standard of care therapeutic approach. A sub-set of NSCLC patients present with tumors whose biology is driven by expression of mutated active forms of ERBB1. Some of the mutant ERBB1 proteins are point mutation mutants and others deletion mutants. Multiple ERBB1 inhibitors are approved to treat this form of the disease including erlotinib, afatinib and recently osimertinib. Osimertinib is a relatively specific inhibitor of mutant active forms of ERBB1 and is at present the standard of care therapeutic. As with all targeted drugs in cancer, eventually NSCLC cells become osimertinib resistant, with diverse mechanisms, including gain of additional ERBB1 mutations or activation of other receptor tyrosine kinases such as c-MET and FGFRs. Overcoming osimertinib resistance remains an important area for the developmental cancer therapeutics field in NSCLC.

The studies here initially determined whether GZ17-6.02 interacted with ERBB1 inhibitors to kill NSCLC cells expressing mutant ERBB1 proteins. Subsequently we determined how GZ17-6.02 killed osimertinib resistant NSCLC cells and interacted with the standard of care agent pemetrexed to further enhance killing.

### Materials and Methods.

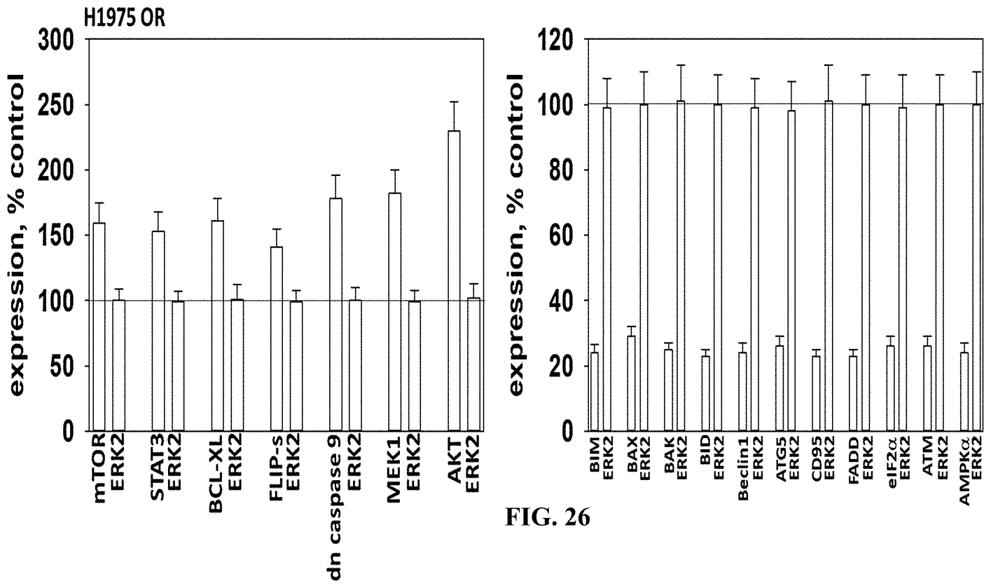

Materials. All human NSCLC lines were obtained from the ATCC (Bethesda, Md.). Lewis Lung Carcinoma cells were obtained from the NCI repository (Bethesda, Md.). Pemetrexed, erlotinib, afatinib and osimertinib were purchased from Selleckchem (Houston, Tex.). Trypsin-EDTA, DMEM, RPMI, penicillin-streptomycin were purchased from GIBCOBRL (GIBCOBRL Life Technologies, Grand Island, N.Y.). Antibodies used: AIF (5318), BAX (5023), BAK (12105), BAD (9239), BIM (2933), BAK1 (12105), Beclin1 (3495), cathepsin B (31718), CD95 (8023), FADD (2782), eIF2a (5324), P-eIF2a S51 (3398), ULK-1 (8054), P-ULK-1 S757 (14202), P-AMPK S51 (2535), AMPKa (2532), P-ATM S1981 (13050), ATM (2873), ATG5 (12994), mTOR (2983), P-mTOR S2448 (5536), P-mTOR S2481 (2974), ATG13 (13468), MCL-1 (94296), BCL-XL (2764), P-AKT T308 (13038), P-ERK1/2 (5726), P-STAT3 Y705 (9145), P-p65 S536 (3033), p⁶² (23214), LAMP2 (49067) all from Cell Signaling Technology (Danvers, Mass.); P-ULK-1 S317 (3803a) was from Abgent; P-ATG13 S318 (19127) from Novus Biologicals. Anti-PD-L1, PD-L2 and MHCA antibodies were from ABCAM (Cambridge, UK). The ODC antibody was purchased from Santa Cruz Biotechnology (Dallas, Tex.). Specific multiple independent siRNAs to knock down the expression of CD95, FADD, Beclin1, ATG5 and eIF2a, and scramble control, were purchased from Qiagen (Hilden Germany). Control studies were presented showing on-target specificity of our siRNAs, primary antibodies and our phospho-specific antibodies to detect both total protein levels and phosphorylated levels of proteins. FIG. 26 shows representative control data showing siRNA protein expression knock down or protein over-expression. Cells were transfected with plasmids to express the indicated proteins or with siRNA molecules to knock down protein expression. Twenty-four h after transfection cells were fixed in place. In cell immunostaining was performed to detect the levels of each protein and in parallel as a loading control, the total expression of invariant ERK2 (n=3+/−SD).

Methods. All cell lines were cultured at 37° C. (5% (v/v CO2) in vitro using RPMI supplemented with dialyzed 5% (v/v) fetal calf serum and 1% (v/v) Non-essential amino acids. Drugs are dissolved in DMSO to make 10 mM stock solutions. The stock solution is diluted to the desired concentration in the media that the cells being investigated grow in. We ensure that the concentration of DMSO is never more than 0.1% (v/v) in the final dilution that is added to cells, to avoid solvent effects. Cells were not cultured in reduced serum media during any study in this manuscript.

Generation of erlotinib, afatinib and osimertinib-resistant cells. Cells were incubated in vitro with increasing concentrations of vehicle control or erlotinib or osimertinib until after ˜6 weeks the HCC827 and H1975 and H1650 cells grew with similar kinetics to sensitive cells in either erlotinib (1 mM) or osimertinib (1 mM), respectively. Afatinib-resistant cells were created in vivo by repeated high dosing until tumors disappeared and then regrew, as previously described.

Assessments of protein expression and protein phosphorylation. Multi-channel fluorescence HCS microscopes perform true in-cell western blotting. Three independent cultures derived from three thawed vials of cells of a tumor were sub-cultured into individual 96-well plates. Twenty-four hours after plating, the cells are transfected with a control plasmid or a control siRNA, or with an empty vector plasmid or with plasmids to express various proteins. After another 24 hours, the cells are ready for drug exposure(s). At various time-points after the initiation of drug exposure, cells are fixed in place using paraformaldehyde and using Triton X100 for permeabilization. Standard immunofluorescent blocking procedures are employed, followed by incubation of different wells with a variety of validated primary antibodies and subsequently validated fluorescent-tagged secondary antibodies are added to each well. The microscope determines the background fluorescence in the well and in parallel randomly determines the mean fluorescent intensity of 100 cells per well. Of note for scientific rigor is that the operator does not personally manipulate the microscope to examine specific cells; the entire fluorescent accrual method is independent of the operator.

For co-localization studies, three to four images of cells stained in the red and green fluorescence channels are taken for each treatment/transfection/condition. Images are approximately 4 MB sized files. Images are merged in Adobe Photoshop CS5 and the image intensity and contrast is then post-hoc altered in an identical fashion inclusive for each group of images/treatments/conditions, so that the image with the weakest intensity is still visible to the naked eye for publication purposes but also that the image with the highest intensity is still within the dynamic range, i.e., not over-saturated.

Detection of cell death by trypan blue assay. Cells were treated with vehicle control or with drugs alone or in combination for 24 h. At the indicated time points cells were harvested by trypsinization and centrifugation. Cell pellets were resuspended in PBS and mixed with trypan blue agent. Viability was determined microscopically using a hemocytometer. Five hundred cells from randomly chosen fields were counted and the number of dead cells was counted and expressed as a percentage of the total number of cells counted.

For plasmids. Cells were plated and 24 h after plating, transfected. Plasmids to express FLIP-s, BCL-XL, dominant negative caspase 9, activated AKT, activated mTOR and activated MEK1 EE were used throughout the study (Addgene, Waltham, Mass.). Empty vector plasmid (CMV) was used as a control. Plasmids expressing a specific mRNA or appropriate empty vector control plasmid (CMV) DNA was diluted in 50 ml serum-free and antibiotic-free medium (1 portion for each sample). Concurrently, 2 ml Lipofectamine 2000 (Invitrogen), was diluted into 50 ml of serum-free and antibiotic-free medium (1 portion for each sample). Diluted DNA was added to the diluted Lipofectamine 2000 for each sample and incubated at room temperature for 30 min. This mixture was added to each well/dish of cells containing 100 ml serum-free and antibiotic-free medium for a total volume of 300 ml, and the cells were incubated for 4 h at 37° C. An equal volume of 2× serum containing medium was then added to each well. Cells were incubated for 24 h, then treated with drugs.

Transfection for siRNA. Cells from a fresh culture growing in log phase as described above, and 24 h after plating transfected. Prior to transfection, the medium was aspirated, and serum-free medium was added to each plate. For transfection, 10 nM of the annealed siRNA or the negative control (a “scrambled” sequence with no significant homology to any known gene sequences from mouse, rat or human cell lines) were used. Ten nM siRNA (scrambled or experimental) was diluted in serum-free media. Four ml Hiperfect (Qiagen) was added to this mixture and the solution was mixed by pipetting up and down several times. This solution was incubated at room temp for 10 min, then added dropwise to each dish. The medium in each dish was swirled gently to mix, then incubated at 37° C. for 2 h. Serum-containing medium was added to each plate, and cells were incubated at 37° C. for 24 h before then treated with drugs (0-24 h).

Assessments of autophagosome and autolysosome levels. Cells were transfected with a plasmid to express LC3-GFP-RFP (Addgene, Watertown Mass.). Twenty-four h after transfection, cells are treated with vehicle control or the drugs alone or in combination. Cells were imaged and recorded at 60× magnification 4 h and 8 h after drug exposure and the mean number of GFP+ and RFP+ punctae per cell determined from >50 randomly selected cells per condition.

Data analysis. Comparison of the effects of various treatments was using one-way ANOVA for normalcy followed by a two tailed Student's t-test. Differences with a p-value of <0.05 were considered statistically significant. Experiments are the means of multiple individual data points per experiment from 3 independent experiments (±SD).

### Results

GZ17-6.02 interacts with ERBB1 inhibitors to kill NSCLC cells expressing mutant active forms of ERBB1. H1650, wild type sensitive and afatinib resistant (AR) H1975 and erlotinib resistant (ER) HCC827 cells were treated with vehicle, erlotinib (500 nM), afatinib (500 nM), GZ17-6.02 (2 mM curcumin final) or the drugs in combination for 24 h. Cell viability was determined by trypan blue exclusion (n=3+/−SD). # p<0.05 greater than GZ17-6.02 alone; ¶ p<0.05 less than corresponding values in drug sensitive cells. In a separate study, cells were treated with vehicle, osimertinib (100 nM), GZ17-6.02 (2 mM curcumin final) or the drugs in combination for 24 h. Cell viability was determined by trypan blue exclusion (n=3+/−SD). # p<0.05 greater than GZ17-6.02 alone; ¶p<0.05 less than corresponding values in drug sensitive cells.

As shown in the data, GZ17-6.02 interacted with erlotinib, afatinib and osimertinib to kill H1975 and H1650 cells that express mutant activated ERBB1 proteins (FIG. 21A-21C). In erlotinib HCC827 cells, the abilities of erlotinib and afatinib to enhance GZ17-6.02 lethality were significantly reduced as was also observed in afatinib resistant H1975 cells (FIGS. 21A and 21). The ability of osimertinib to enhance the efficacy of GZ17-6.02 was also reduced in afatinib- and erlotinib resistant cells (FIG. 21C).

Osimertinib-resistant H1975 and H1650 cells were generated as described and studied to determine if GZ17-6.02 interacts with pemetrexed to kill NSCLC cells. H1975 and H1650 cells (wild type sensitive and osimertinib resistant (OR)) were treated with vehicle, erlotinib (500 nM), afatinib (500 nM), osimertinib (100 nM), GZ17-6.02 (2 mM curcumin final) or the drugs in combination for 24 h. Cell viability was determined by trypan blue exclusion (n=3+/−SD). # p<0.05 greater than GZ17-6.02 alone; ¶ p<0.05 less than corresponding values in drug sensitive cells. In a separate study, the cells were treated with vehicle, pemetrexed (500 nM), GZ17-6.02 (2 mM curcumin final) or the drugs in combination for 24 h. Cell viability was determined by trypan blue exclusion (n=3+/−SD). # p<0.05 greater than GZ17-6.02 alone; ¶ p<0.05 less than corresponding values in drug sensitive cells. In further study, NSCLC cells were treated with vehicle, pemetrexed (500 nM), GZ17-6.02 (2 mM curcumin final) or the drugs in combination for 24 h. Cell viability was determined by trypan blue exclusion (n=3+/−SD). # p<0.05 greater than GZ17-6.02 alone. The mutational status of K-/N-RAS or of ERBB1 is noted in each graph.

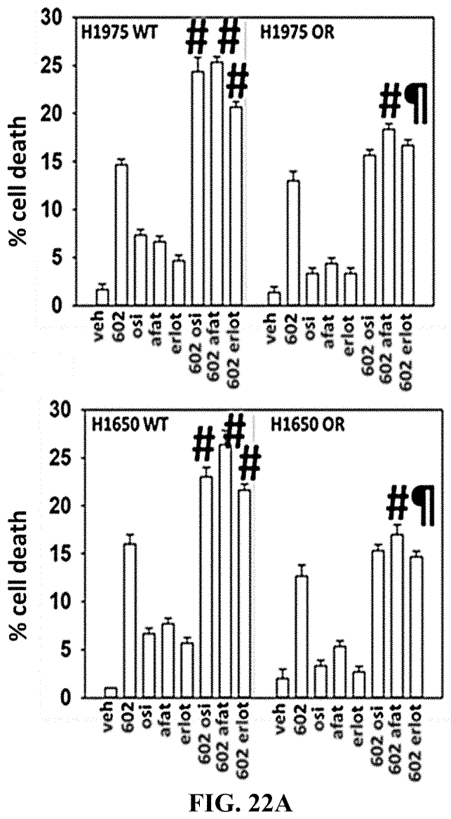

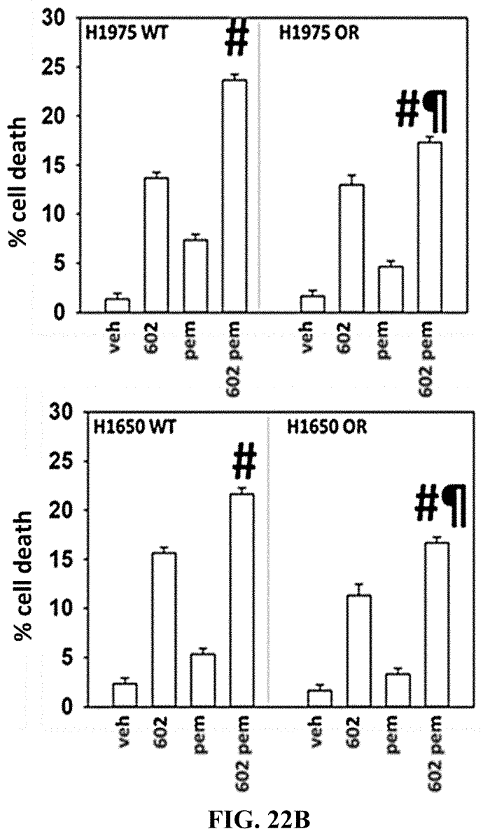

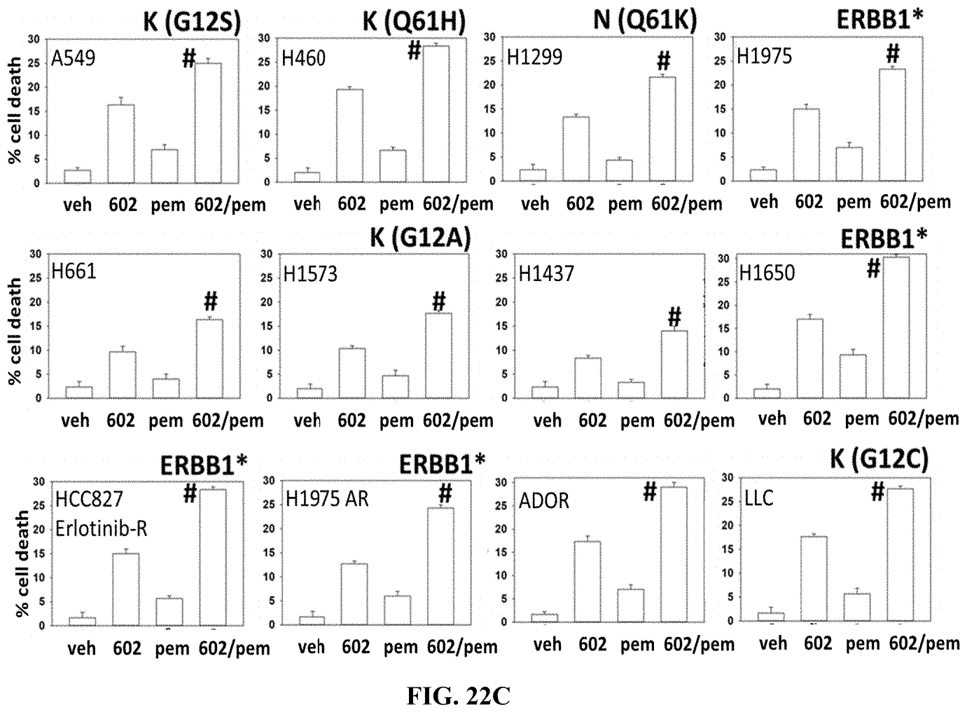

As shown in the data, in osimertinib-resistant cells the abilities of erlotinib and osimertinib to enhance GZ17-6.02 killing were abolished, with only afatinib capable of modestly enhancing tumor cell killing (FIG. 22A). We next determined whether GZ17-6.02 could interact with the NSCLC therapeutic pemetrexed to kill wild type and osimertinib-resistant cells. As noted in panel A, osimertinib resistance weakly reduced the efficacy of GZ17-6.02 as a single agent, and it interacted to kill both wild type and osimertinib-resistant cells, albeit with a lesser efficacy in the resistant cells (FIG. 22B). We then determined whether GZ17-6.02 interacted with pemetrexed to kill other NSCLC cell lines; regardless of mutant RAS or ERBB1 expression, GZ17-6.02 and pemetrexed interacted to kill (FIG. 22C).

We then determined the alterations in cellular signaling and protein expression in NSCLC cells treated with GZ17-6.02 and either osimertinib or pemetrexed. GZ17-6.02 interacted with osimertinib in wild type H1975 cells to activate ATM, the AMPK, ULK1, ATG13 and PERK (Supplemental Tables 1 and 2). The drugs interacted to cause inactivation of mTORC1, mTORC2, eIF2a, MEK1/2, ERK1/2, AKT, JAK2, STAT3, STAT5, ERBB1, PDGFRb, c-MET, p70 S6K, c-SRC, NFkB, JNK1/2, YAP and TAZ. The drug combination increased protein MHCA expression and reduced the levels of PD-L1, IDO1, HDAC1, HDAC2, HDAC3, HDAC4, HDAC6, and HDAC7. Similar findings were made in H1650 cells. In afatinib-resistant H1975 cells, the drug combination caused significantly more ERK1/2 inactivation and did not inactivate p70 S6K or STAT5 and caused a compensatory increase in c-KIT survival signaling (that was not observed in osimertinib-resistant cells).

Based on our prior studies with GZ17-6.02 we predicted that the inactivation of mTOR, the activation of ULK1 and increased ATG13 S318 phosphorylation would cause autophagosome formation. We found that resistance to ERBB1 inhibitors is associated with a reduced ability to form autophagosomes. H1975 (wild type sensitive and afatinib-resistant (AR)) were transfected to express LC3-GFP-RFP and subsequently treated with vehicle, osimertinib (100 nM), GZ17-6.02 (2 mM curcumin final) or the drugs in combination for 4 h and 8 h. The number of intense staining GFP+ and RFP+ punctae were determined randomly in at least 50 cells and the mean number of punctae per cell determined (n=3+/−SD). # p<0.05 greater than GZ17-6.02 value; ¶ p<0.05 greater than corresponding value after 4 h; ˜ p<0.05 less than corresponding value in wild type sensitive cells. Erlotinib-resistant HCC827 cells were transfected with siRNA molecules to knock down protein levels or with plasmids to express activated forms of mTOR or STAT3 and then subsequently treated with vehicle or [osimertinib (100 nM)+GZ17-6.02 (2 mM curcumin final)] in combination for 4 h and 8 h. The number of intense staining GFP+ and RFP+ punctae were determined randomly in at least 50 cells and the mean number of punctae per cell determined (n=3+/−SD). ¶ p<0.05 greater than corresponding value after 4 h; * p<0.05 less than corresponding values in siSCR/CMV transfected cells. Afatinib-resistant H1975 cells were transfected with siRNA molecules to knock down protein levels or with plasmids to express activated forms of mTOR or STAT3 and then subsequently treated with vehicle or [osimertinib (100 nM)+GZ17-6.02 (2 mM curcumin final)] in combination for 4 h and 8 h. The number of intense staining GFP+ and RFP+ punctae were determined randomly in at least 50 cells and the mean number of punctae per cell determined (n=3+/−SD). ¶ p<0.05 greater than corresponding value after 4 h; * p<0.05 less than corresponding values in siSCR/CMV transfected cells.

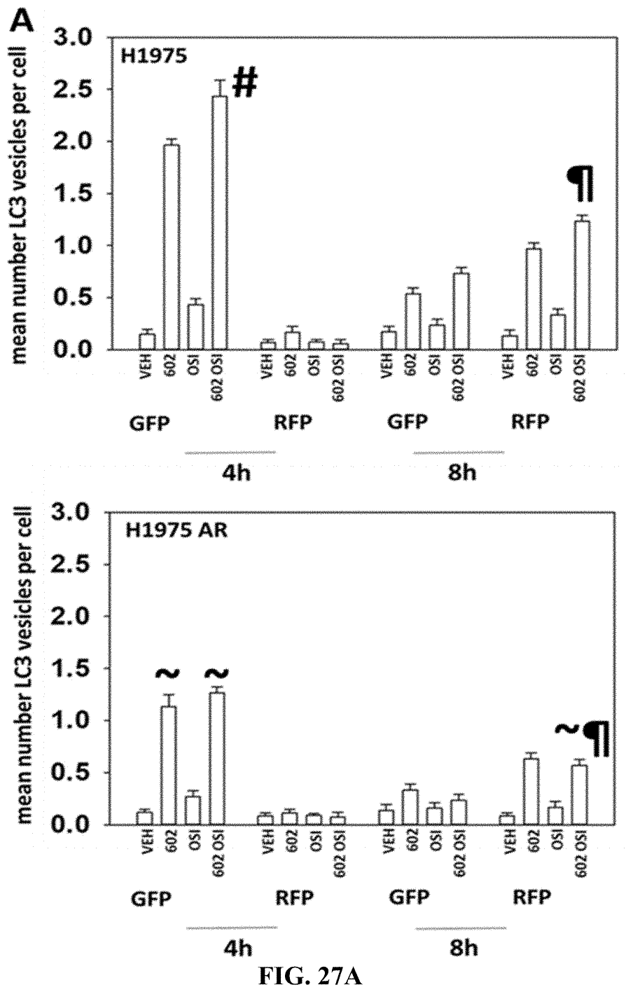

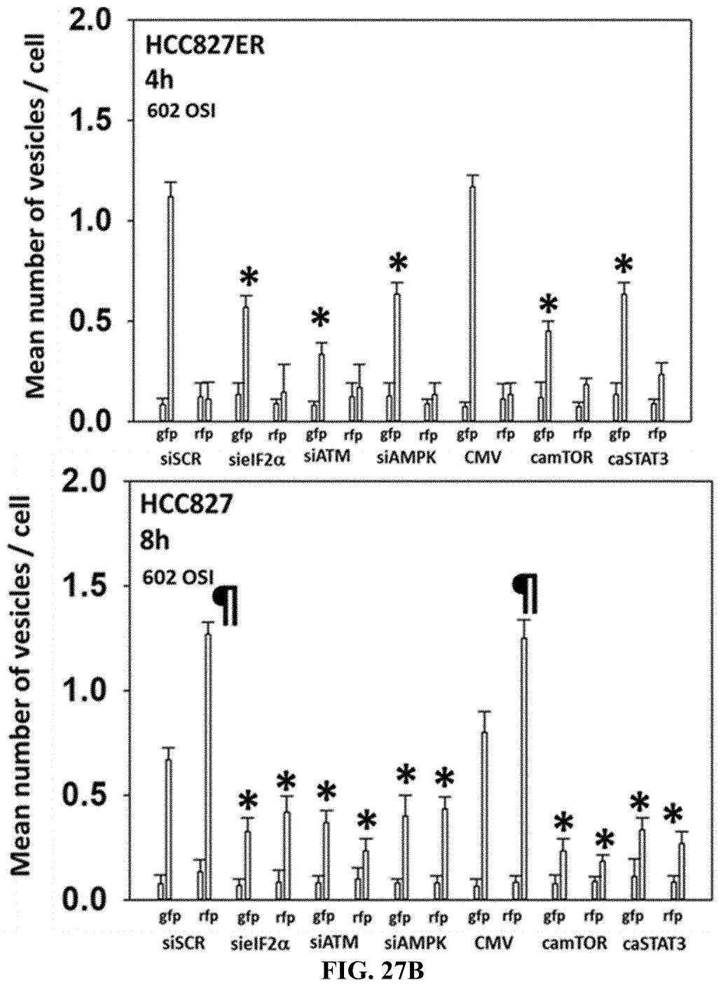

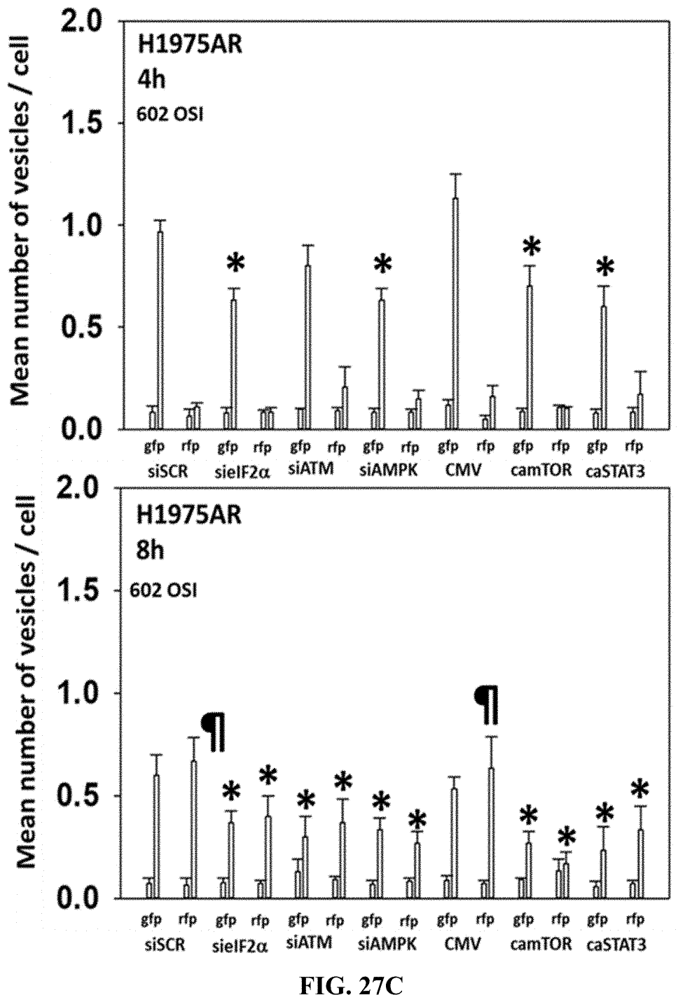

As shown in the data, GZ17-6.02 interacted with osimertinib in an additive fashion to increase autophagosome formation and subsequently autophagosome formation (FIG. 27A, upper graph). In afatinib-resistant H1975 cells, GZ17-6.02 enhanced autophagosome formation to a lesser extent than in wild type sensitive cells and did not further interact with osimertinib (lower graph). Increasing numbers of autolysosomes were also observed 8 h after treatment, but again, this value was lower than that observed in the sensitive cells. We next determined the relative role of altered cellular signaling processes in autophagosome formation and autophagic flux. Knock down of ATM, AMPKa, eIF2a or expression of activated mTOR or activated STAT3 significantly suppressed autophagosome formation and autophagic flux (FIGS. 27B and 27C).

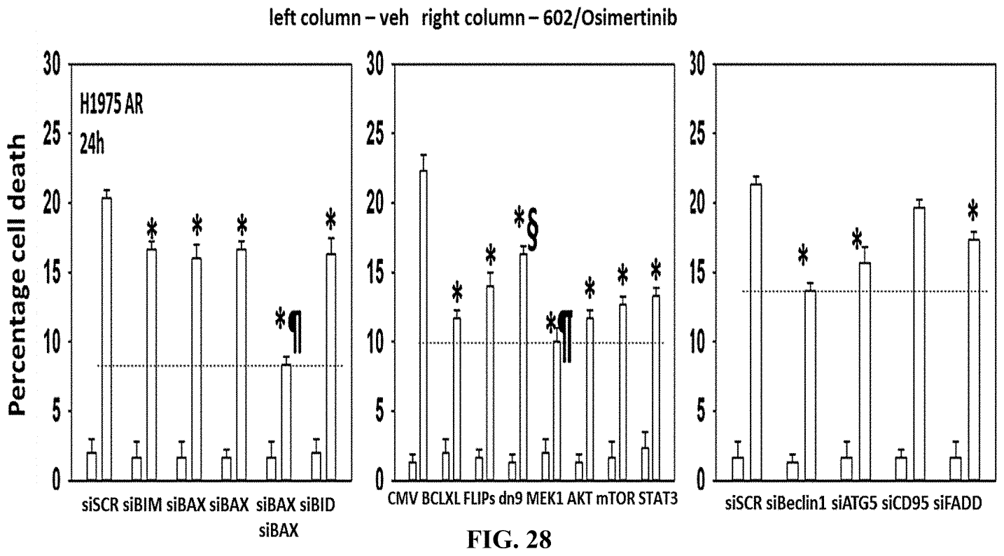

The killing of afatinib-resistant NSCLC cells requires [BAX+BAK] and autophagosome formation and is significantly reduced by expression of activated AKT, activated mTOR or activated MEK1. Afatinib-resistant H1975 cells were transfected with siRNA molecules to knock down protein expression or with plasmids to express regulatory proteins. Subsequently, cells were treated with vehicle or [osimertinib (100 nM)+GZ17-6.02 (2 mM curcumin final)] in combination for 24 h. Cell viability was determined by trypan blue exclusion (n=3+/−SD). * p<0.05 less than corresponding siSCR/CMV value; ¶ p<0.05 less than corresponding values in all other conditions; § p<0.05 greater than corresponding values in all other manipulated conditions. As shown in the data, knock down of [BAX+BAK], Beclin1, ATG5 or FADD significantly reduced cell killing by [GZ17-6.02+osimertinib] (FIG. 28).

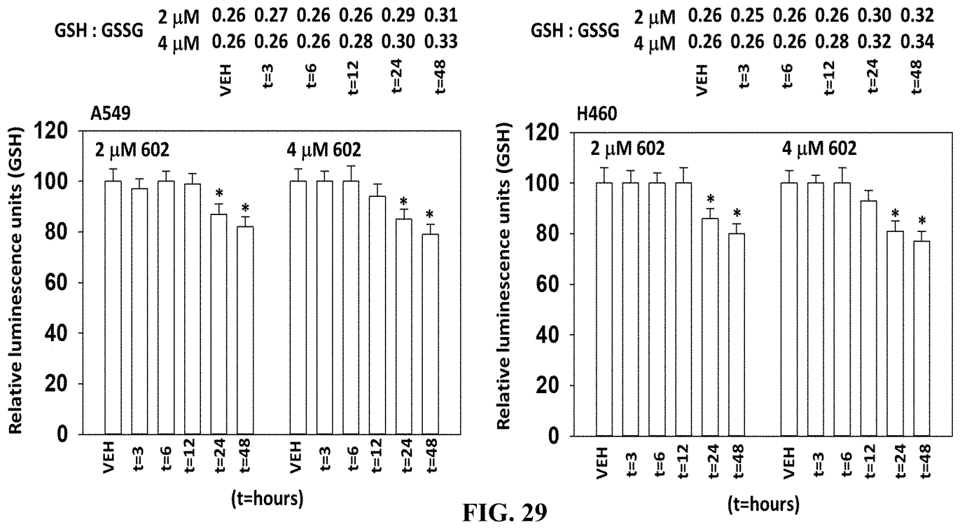

GZ17-6.02 weakly alters GSH levels and the GSH:GSSG ratio in NSCLC cells. Cells were treated with vehicle control or with GZ17-6.02 (2 mM or 4 mM curcumin final concentration). Cells were isolated 3 h-48 h afterwards and the total levels of GSH expressed as a percentage of vehicle control at each time point and the ratio of GSH to GSSG determined using a kit purchased from Promega. (n=3+/−SD) * p<0.05 less than vehicle control value. As shown in the data, the total levels of GSH and the GSH:GSSG ratio were not significantly altered by GZ17-6.02 over 12 h (FIG. 29). Modest significant reductions in the levels of GSH and alterations in the ratio were observed after 24-48 h, however there was no clear dose-dependency comparing the two GZ17-6.02 concentrations. These data imply that autophagy, death receptor signaling, and mitochondrial dysfunction play key roles in the cell killing caused by the drug combination, with altered redox potential unlikely to play any role. Of note was that expression of dominant negative caspase 9 relatively weakly prevented cell death compared to other interventions arguing that non-apoptotic processes downstream of the mitochondrion played key roles.

Based on our viability data with GZ17-6.02 and pemetrexed in ERBB1 inhibitor resistant NSCLC cells, we compared and contrasted the ability of the drug combination to alter signaling and protein expression in H1975 cells; wild type sensitive; afatinib-resistant; osimertinib-resistant. Regardless of drug resistance, the drug combination activated ATM, AMPK, ULK1, ATG13 and PERK. The combination inactivated ERBB1, ERBB2, mTORC1, mTORC2, eIF2a, AKT, ERK1/2, JAK2, STAT3, STAT5, p70 S6K, NFkB, c-SRC, c-MET, and c-KIT. The combination increased the expression of Beclin1, ATG5, and FAS-L and reduced the expression of BCL-XL and MCL1 (Supplemental Tables 3 and 4). Regardless of osimertinib resistance, the drug combination reduced the protein levels of HDAC2, HDAC3 and HDAC6 (Supplemental Table 5). In prior work, we have linked reduced expression of HDAC2 and HDAC3 to increased expression of the immunotherapy biomarker MHCA and reduced levels of PD-L1. In multiple NSCLC lines the drug combination significantly reduced expression of PD-L1, ODC and IDO1 and elevated MHCA levels (Supplemental Table 6).

Next, H1975 (wild type sensitive and afatinib-resistant (AR)) were transfected to express LC3-GFP-RFP and subsequently treated with vehicle, pemetrexed (500 nM), GZ17-6.02 (2 mM curcumin final) or the drugs in combination for 4 h and 8 h. The number of intense staining GFP+ and RFP+ punctae were determined randomly in at least 50 cells and the mean number of punctae per cell determined (n=3+/−SD). # p<0.05 greater than GZ17-6.02 value; ¶ p<0.05 greater than corresponding value after 4 h; ˜ p<0.05 less than corresponding value in wild type sensitive cells. Afatinib-resistant H1975 cells were also transfected with siRNA molecules to knock down protein levels or with plasmids to express activated forms of mTOR or STAT3 and then subsequently treated with vehicle or [pemetrexed (500 nM)+GZ17-6.02 (2 mM curcumin final)] in combination for 4 h and 8 h. The number of intense staining GFP+ and RFP+ punctae were determined randomly in at least 50 cells and the mean number of punctae per cell determined (n=3+/−SD). ¶ p<0.05 greater than corresponding value after 4 h; * p<0.05 less than corresponding values in siSCR/CMV transfected cells. Osimertinib-resistant H1975 cells were transfected with siRNA molecules to knock down protein levels or with plasmids to express activated forms of mTOR or STAT3 and then subsequently treated with vehicle or [pemetrexed (500 nM)+GZ17-6.02 (2 mM curcumin final)] in combination for 4 h and 8 h. The number of intense staining GFP+ and RFP+ punctae were determined randomly in at least 50 cells and the mean number of punctae per cell determined (n=3+/−SD). ¶ p<0.05 greater than corresponding value after 4 h; ˜˜ p<0.05 less than corresponding value in afatinib-resistant H1975 cells; * p<0.05 less than corresponding values in siSCR/CMV transfected cells.

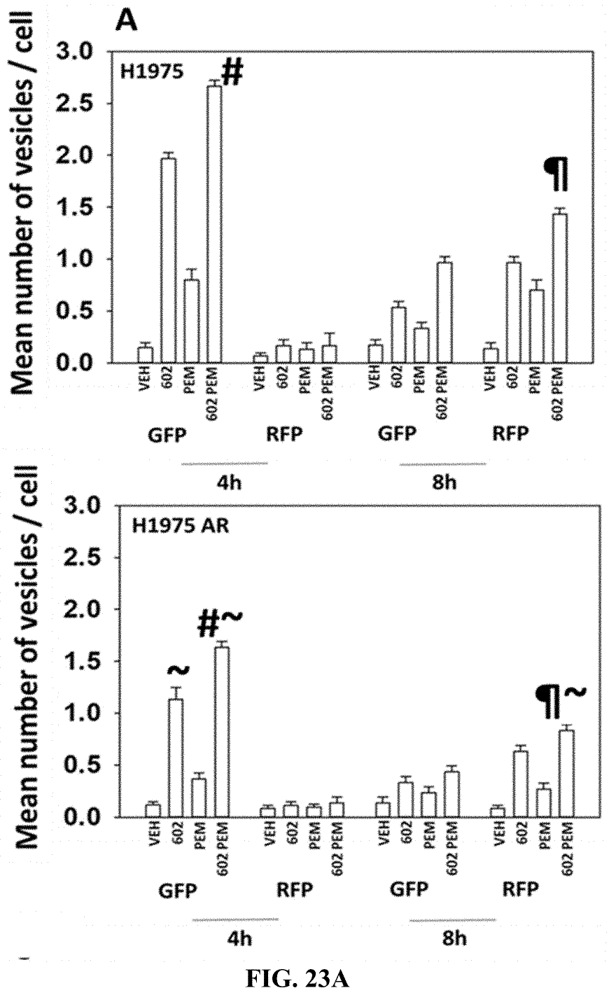

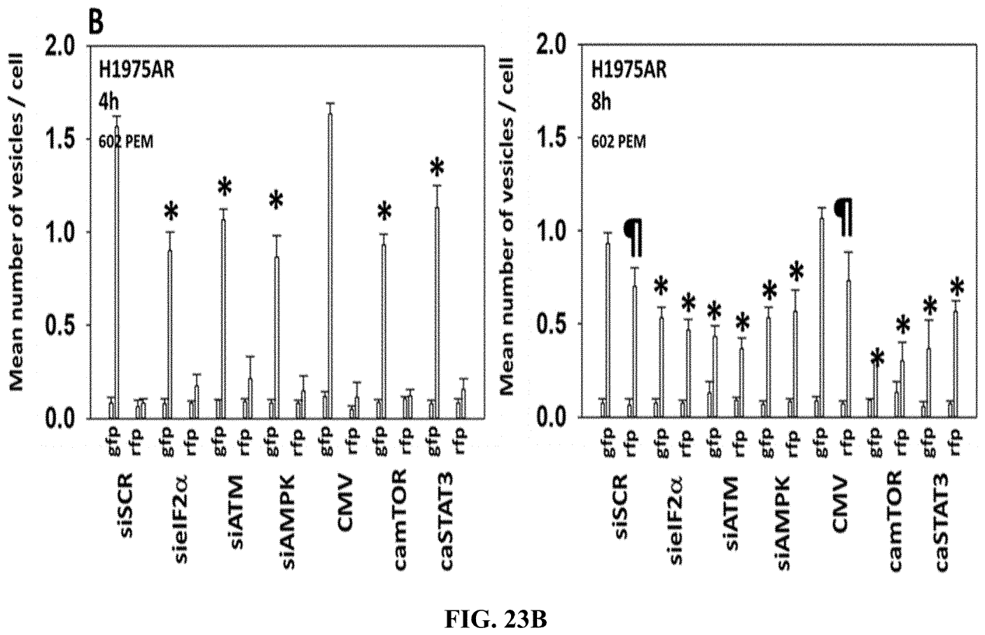

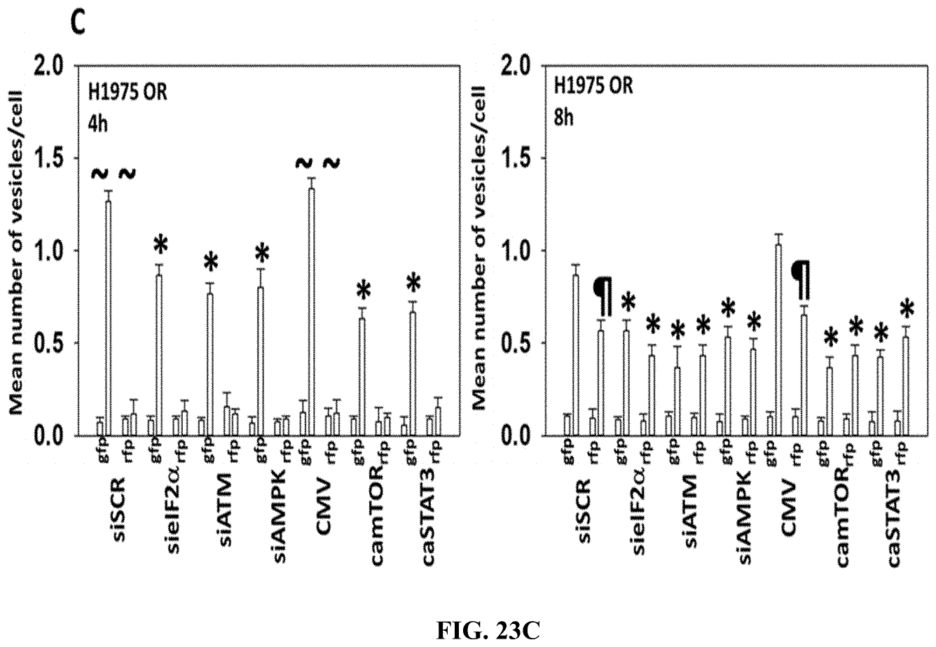

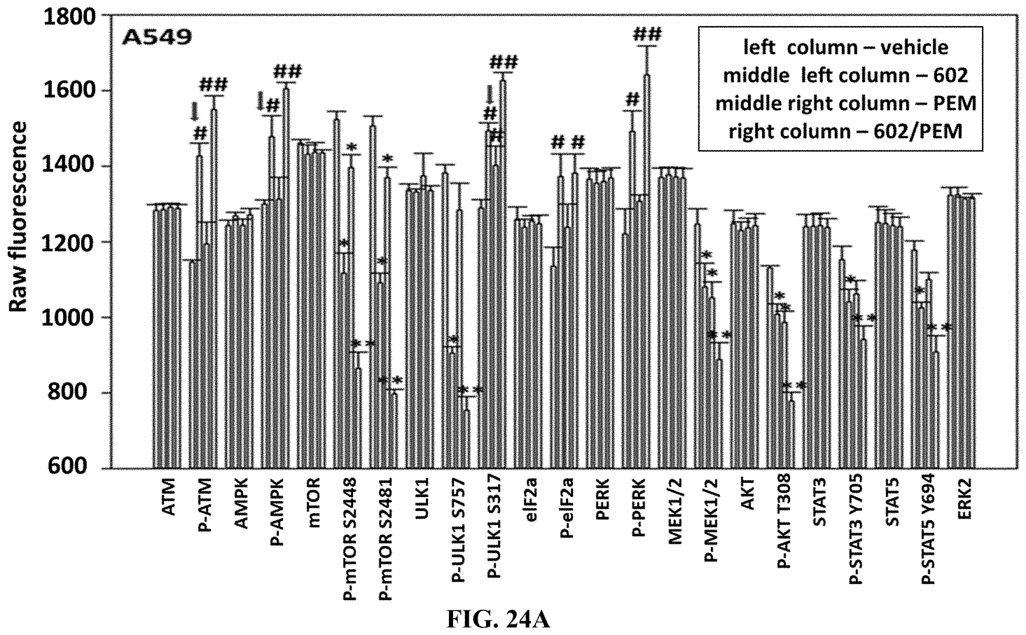

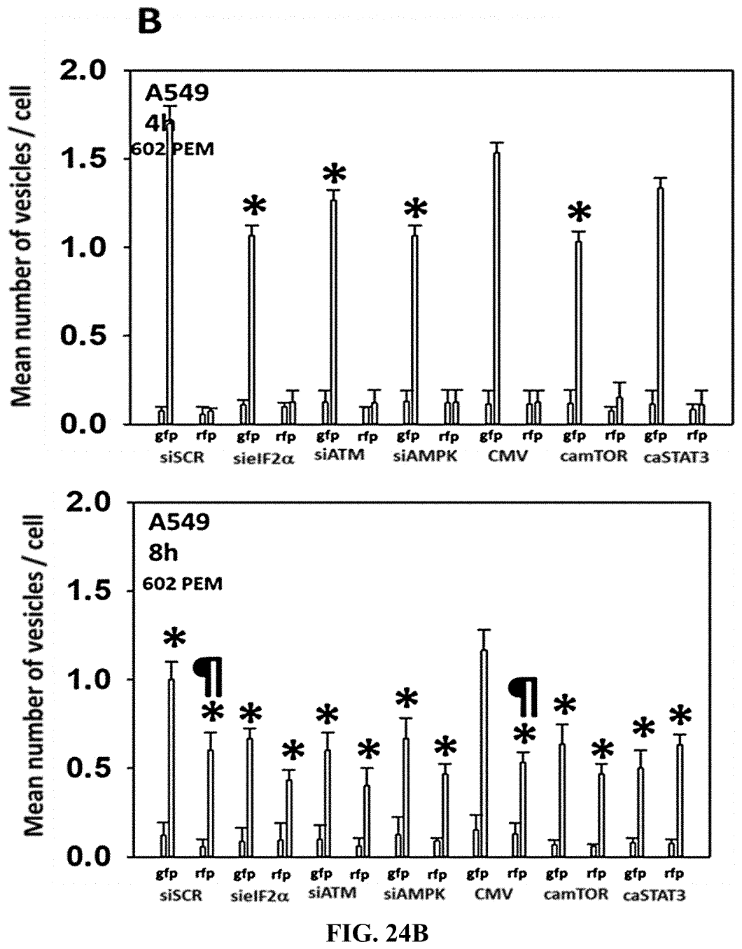

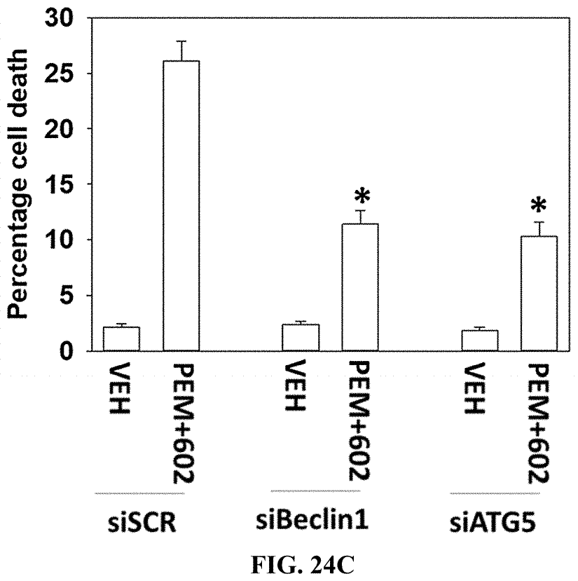

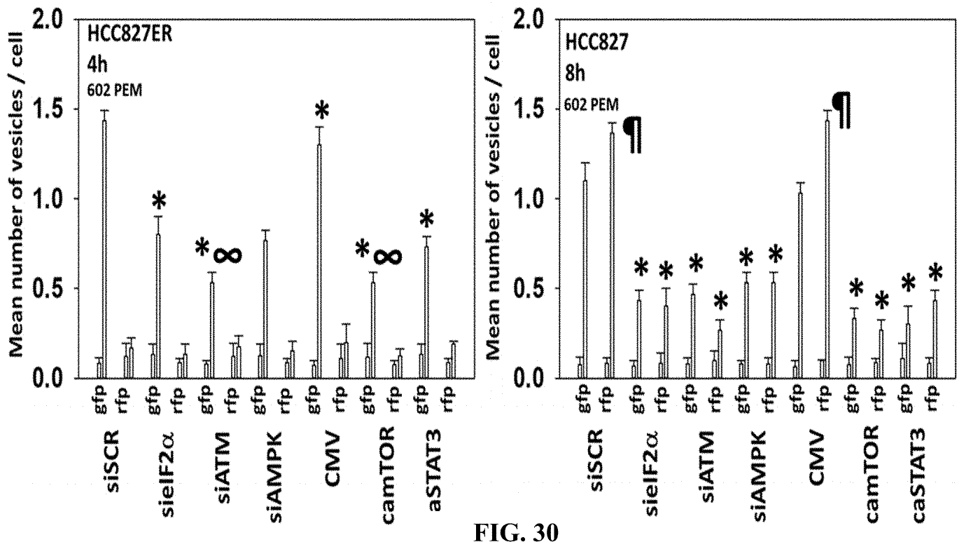

As shown in the data, GZ17-6.02 interacted with pemetrexed in an additive fashion to increase autophagosome formation and to cause autophagic flux (FIG. 23A). The drug combination caused significantly less autophagosome formation and autophagic flux in the afatinib resistant cells. The ability of afatinib-resistant cells to form autophagosomes after drug exposure was significantly reduced by knock down of eIF2a, ATM or AMPKa or by expression of activated mTOR or activated STAT3 (FIG. 23B). The ability of [GZ17-6.02+pemetrexed] to cause autophagosome formation in the osimertinib-resistant cells was significantly lower than that found in wild type sensitive or afatinib-resistant cells (FIG. 23C). Autophagosome formation in the osimertinib-resistant cells was also significantly reduced by knock down of eIF2a, ATM or AMPKa or by expression of activated mTOR or activated STAT3. In contrast to our autophagosome data, the drug-induced levels of autolysosomes in the afatinib-resistant and osimertinib-resistant cells were not significantly different. Similar alterations in cell signaling, autophagy and viability data were obtained treating A549 NSCLC cells with the drug combination which express a mutant K-RAS protein and erlotinib-resistant HCC827 cells (FIG. 24; FIG. 30). In erlotinib-resistant HCC827 cells signaling by ATM enhances autophagosome formation whereas signaling from mTOR suppresses this event. Erlotinib-resistant HCC827 were transfected with siRNA molecules to knock down protein levels or with plasmids to express activated forms of mTOR or STAT3 and then subsequently treated with vehicle or [pemetrexed (500 nM)+GZ17-6.02 (2 mM curcumin final)] in combination for 4 h and 8 h. The number of intense staining GFP+ and RFP+ punctae were determined randomly in at least 50 cells and the mean number of punctae per cell determined (n=3+/−SD). ¶ p<0.05 greater than corresponding value after 4 h; * p<0.05 less than corresponding values in siSCR/CMV transfected cells; ∞ p<0.05 less than values in sieIF2a, siAMPKa and caSTAT3.

In contrast to the other lines tested, the HCC827 line exhibited a strong dependence on altered signaling by ATM and mTOR to stimulate autophagosome formation.

A549 cells were treated with vehicle control, GZ17-6.02 (2 mM final curcumin), pemetrexed (500 nM) or the drugs combined for 6 h. Cells were fixed in place and immunostaining performed to determine protein expression and phosphorylation (n=3+/−SD) * p<0.05 less than vehicle; ** p<0.05 less than GZ17-6.02 alone; # p<0.05 greater than vehicle control; ## p<0.05 greater than GZ17-6.02 alone. A549 cells were transfected with siRNA molecules to knock down protein levels or with plasmids to express activated forms of mTOR or STAT3 and then subsequently treated with vehicle or [pemetrexed (500 nM)+GZ17-6.02 (2 mM curcumin final)] in combination for 4 h and 8 h. The number of intense staining GFP+ and RFP+ punctae were determined randomly in at least 50 cells and the mean number of punctae per cell determined (n=3+/−SD). ¶ p<0.05 greater than corresponding value after 4 h; * p<0.05 less than corresponding values in siSCR/CMV transfected cells. A549 cells were transfected to knock down Beclin1 or ATG5 expression. Subsequently cells were treated with vehicle, pemetrexed (500 nM), GZ17-6.02 (2 mM curcumin final) or the drugs in combination for 24 h. Cell viability was determined by trypan blue exclusion (n=3+/−SD). * p<0.05 less than corresponding value in siSCR cells.

Afatinib-resistant and osimertinib-resistant H1975 cells were transfected with siRNA molecules to knock down protein expression or with plasmids to express regulatory proteins. Subsequently, cells were treated with vehicle or [pemetrexed (500 nM)+GZ17-6.02 (2 mM curcumin final)] in combination for 24 h. Cell viability was determined by trypan blue exclusion (n=3+/−SD). * p<0.05 less than corresponding siSCR/CMV value; ** p<0.05 less than corresponding values in afatinib-resistant cells; ¶ p<0.05 less than corresponding values in all other conditions; § p<0.05 greater than corresponding values in all other manipulated conditions.

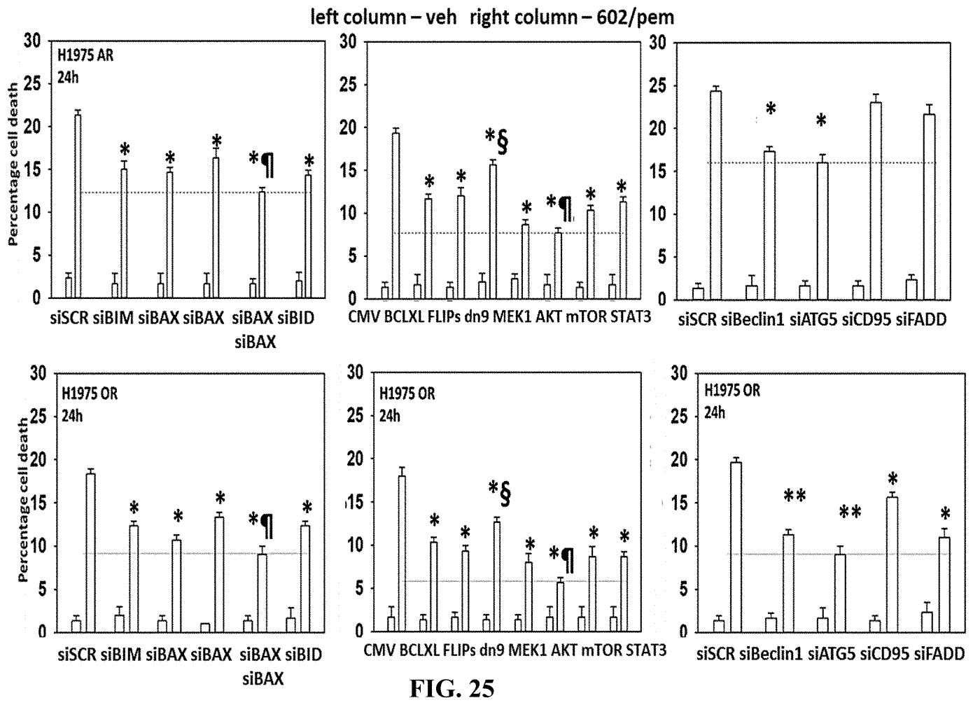

As shown in the data, the ability of [GZ17-6.02+pemetrexed] to kill osimertinib-resistant cells trended lower than the ability of the drug combination to kill afatinib-resistant cells (FIG. 24 and FIG. 25). Combined knock down of BAX and BAK significantly reduced killing in both the afatinib-resistant and the osimertinib-resistant cells by ˜50% with knock down of BID reducing death by ˜35%. In both resistant cell types activated AKT and to a lesser extent activated MEK1, activated STAT3 or activated mTOR significantly reduced killing. Knock down of Beclin1 or ATG5 was significantly more protective in osimertinib-resistant cells compared to afatinib-resistant cells. Death receptor signaling also trended to be more important in the killing processes in osimertinib-resistant cells than in afatinib-resistant cells. Expression of dominant negative caspase 9 was less protective than over-expression of FLIP-s or BCL-XL in both resistant lines arguing that cell execution downstream of the mitochondrion was largely non-apoptotic.

### Discussion

The development of drug resistance in NSCLC tumors expressing mutant active forms of ERBB1 is a major problem in prolonging patient quality of life and survival. The present studies were designed to define the biology of GZ17-6.02 in NSCLC cells expressing mutant active ERBB1 proteins and to define whether it could overcome resistance to afatinib or osimertinib. GZ17-6.02 interacted with erlotinib, afatinib or osimertinib to kill NSCLC cells expressing mutant ERBB1. However, in cells made resistant to either afatinib or osimertinib, GZ17-6.02 could not subvert the resistant phenotype. Based on those findings, we then determined whether GZ17-6.02 interacted with the NSCLC therapeutic pemetrexed to kill. Resistance to ERBB1 inhibitors only modestly reduced the efficacy of GZ17-6.02 and caused only a ˜20% reduction in the lethal interaction between GZ17-6.02 and pemetrexed.

When we examined drug-induced changes in cell signaling in the sensitive and ERBB1 inhibitor resistant cells, combining GZ17-6.02 with either osimertinib or pemetrexed, their responses exhibited subtle rather than profound differences. For example, from over 20 parameters measured, the major observation for afatinib-resistant cells treated with [GZ17-6.02+osimertinib] was that the drug combination caused significantly more ERK1/2 inactivation in sensitive cells and did not inactivate p70 S6K or STAT5 and that it caused a compensatory increase in c-KIT survival signaling in resistant cells. The complex milieux of signaling trends collectively resulted in the outcomes of afatinib-resistant cells being less capable to form autophagosomes and to die.

Because our initial hypotheses were incorrect regarding the hope that GZ17-6.02 would abolish ERBB1 inhibitor resistance, we then performed studies to define the interactions of GZ17-6.02 with the standard of care therapeutic pemetrexed in the NSCLC cells. We specifically chose pemetrexed rather than carboplatin because via DNA damage signaling pemetrexed causes ATM activation and by increasing the intracellular concentration of ZMP, and analogue of AMP, it causes allosteric activation of the AMPK [27-29]. In wild type sensitive cells compared to osimertinib-resistant cells, [GZ17-6.02+pemetrexed] signaling trended to cause greater inactivation of ERBB1 and ERBB2 whereas in the osimertinib-resistant cells greater ERBB4 and c-MET inactivation was observed.

Regardless of ERBB1 inhibitor resistance, [GZ17-6.02+pemetrexed] inactivated AKT, mTORC1 and mTORC2 to a similar extent. Although the amount of drug-induced ATG13 S318 phosphorylation induced was also identical regardless of drug resistance, as were the increased levels of Beclin1 and ATG5. Nevertheless, afatinib-resistant H1975 cells were significantly less efficient at forming autophagosomes than wild type sensitive cells, a ˜55% reduction, and osimertinib-resistant cells exhibited a further significant reduction in autophagosome formation compared to the afatinib-resistant cells. Both afatinib- and osimertinib-resistant cells exhibited similar levels of subsequent autolysosome formation which was ˜30% of that observed in the sensitive cells. These data argue that the “defect” in the drug-resistant cells is specifically related to autophagosome formation rather than the abilities of cells to promote autophagic flux and subsequent autolysosome formation. One potential mechanism by which autophagosome formation could be disrupted is via the sequestration of Beclin1 by protective BH3 domain proteins such as BCL-XL and MCL1. However, data from Supplementary Table 4 demonstrated that the drug-resistant cells under basal conditions only expressed 10-20% greater levels of BCL-XL than were found in the sensitive cells.

In conclusion, in vitro and in vivo, GZ17-6.02 and pemetrexed interact to suppress the growth of osimertinib resistant NSCLC cells and to prolong animal survival. Additional in vitro screening studies, beyond examination of Beclin1 and ATG5 should be undertaken.

The data in supplemental tables 1 and 2 shows the impact of GZ17-6.02 as a single agent and when combined with osimertinib in parental “wild type” H1975 cells and in H1975 cells made resistant to the EGFR inhibitor afatinib. Data are also presented for parental “wild type” H1650 cells. Regardless of afatinib resistance, GZ17-6.02 as a single agent and more so when combined with osimertinib activated ATM-AMPK signaling which causes the inactivation of mTOR and activation of ULK1 and ATG13 phosphorylation which is the initiating signal for autophagosome formation. GZ17-6.02 and osimertinib combined to inactivate c-SRC, AKT, ERK1/2 and p70 S6K and GZ17-6.02 as a single agent and more so when combined with osimertinib caused an endoplasmic reticulum stress response. These events were associated with enhanced expression of the autophagy proteins Beclin1 and ATG5 and reduced expression of cytoprotective MCL-1 and BCL-XL. The drug combination inactivated the Hippo pathway and likely enhanced the immunogenicity of the cells as judged by increased expression of MHCA and decreased expression of IDO1.

Supplemental table 3 shows the impact of GZ17-6.02 as a single agent and when combined with the NSCLC therapeutic pemetrexed in parental “wild type” H1975 cells, in H1975 cells made resistant to the EGFR inhibitor afatinib, and in H1975 cells made resistant to the mutant EGFR osimertinib. Regardless of resistance to afatinib and osimertinib, GZ17-6.02 as a single agent and more so when combined with pemetrexed activated ATM-AMPK signaling which causes the inactivation of mTOR and activation of ULK1 and ATG13 phosphorylation which is the initiating signal for autophagosome formation. GZ17-6.02 and pemetrexed combined to inactivate ERBB1, ERBB2, ERBB3, ERBB4, c-SRC, AKT, ERK1/2 and p70 S6K and GZ17-6.02 as a single agent and more so when combined with pemetrexed caused an endoplasmic reticulum stress response. These events were associated with enhanced expression of the autophagy proteins Beclin1 and ATG5 and as shown in supplemental table 4 reduced expression of cytoprotective MCL-1 and BCL-XL. The drug combination inactivated the Hippo pathway and likely enhanced the immunogenicity of the cells as judged by increased expression of MHCA and decreased expression of IDO1.

Data in supplemental table 2 and supplemental tables 5 and 6 show the impact of GZ17-6.02 combined with osimertinib or with pemetrexed on the expression of HDAC proteins. GZ17-6.02 as a single agent reduced the expression of HDAC6 and this effect was enhanced by either osimertinib or pemetrexed. This also correlates with inactivation of multiple intracellular signaling pathways. Similar effects across the various resistant NSCLC cells were observed for HDAC2 and HDAC3. Prior data has shown that HDAC2 and HDAC3 play key roles in the regulation of PD-L1 and MHCA expression.

SUPPLEMENTAL TABLE 1
The impact of GZ17-6.02 and osimertinib on cell signaling in NSCLC
cells (part 1). Cells were treated wit h ve hicle control, GZ17-6.02 (2 mM final curcumin),
osimertinib (100 nM) or the drugs combined for 6 h. Cells were fixed in place and immunostaining
performed to determine protein expression and phosphorylation (n = 3 +/− SD) * p < 0.05 less t han
ve hicle; # p < 0.05 greater than vehicle.

| Vehicle | 1975 WT | | | 1975 AR | | | 1650 WT | | |
|---|---|---|---|---|---|---|---|---|---|
| 100% | 602 | OSI | 6 + O | 602 | OSI | 6 + O | 602 | OSI | 6 + O |
| P-ATM | 112 | 100 | 119# | 112 | 101 | 116# | 116# | 98 | 118# |
| S1980 | | | | | | | | | |
| P- | 108 | 101 | 114# | 108 | 102 | 115# | 119# | 98 | 120# |
| AMPKα | | | | | | | | | |
| T172 | | | | | | | | | |
| P-mTOR | 74* | 68* | 56* | 69* | 66* | 60* | 85* | 96 | 83* |
| S2448 | | | | | | | | | |
| P-mTOR | 69* | 68* | 60* | 68* | 71* | 60* | 77* | 87* | 76* |
| S2481 | | | | | | | | | |
| P-ULK1 | 68* | 63* | 58* | 63* | 65* | 53** | 77* | 81* | 71* |
| S757 | | | | | | | | | |
| P-ULK1 | 108 | 105 | 113# | 106 | 99 | 118# | 109 | 107 | 114# |
| S317 | | | | | | | | | |
| P-PERK | 118# | 109 | 120# | 117# | 111 | 124# | 114# | 104 | 114# |
| T980 | | | | | | | | | |
| P-eIF2α | 115# | 109 | 116# | 116# | 105 | 121# | 117# | 114 | 122# |
| S51 | | | | | | | | | |
| P- | 93 | 83* | 76* | 93 | 82* | 76* | 92 | 97 | 87* |
| MEK1/2 | | | | | | | | | |
| P-AKT | 91 | 81* | 70* | 94 | 86* | 80* | 95 | 90 | 85* |
| T308 | | | | | | | | | |
| P-STAT3 | 93 | 92 | 85* | 92 | 92 | 81* | 96 | 95 | 94 |
| Y705 | | | | | | | | | |
| P-STAT5 | 89 | 88 | 82* | 93 | 95 | 92 | 95 | 98 | 93 |
| Y694 | | | | | | | | | |
| Beclin1 | 110 | 113# | 116# | 106 | 109 | 115# | 111 | 103 | 115# |
| ATG5 | 109 | 111 | 117# | 107 | 105 | 113# | 116# | 106 | 117# |
| P-ATG13 | 124# | 126# | 129# | 118# | 121# | 126# | 123# | 115# | 129# |
| S318 | | | | | | | | | |
| GRP78 | 121# | 119# | 121# | 115# | 103# | 116# | 120# | 111 | 122# |
| CHOP | 114# | 109 | 115# | 110 | 102 | 113# | 111 | 106 | 116# |
| PP1 | 116# | 104 | 120# | 119# | 112 | 125# | 119# | 103 | 122# |
| NOXA | 106 | 100 | 107 | 104 | 100 | 106 | 111 | 101 | 113# |
| PUMA | 109 | 99 | 109 | 105 | 98 | 107 | 109 | 100 | 113# |
| FLIP-s | 92 | 85* | 84* | 90 | 79* | 75* | 93 | 102 | 90 |
| ERBB1 | 95 | 99 | 92 | 100 | 100 | 105 | 100 | 98 | 96 |
| P-B1 | 94 | 73* | 71* | 93 | 76* | 73* | 71* | 61* | 58* |
| ERBB2 | 98 | 100 | 98 | 101 | 102 | 92 | 95 | 94 | 91 |
| P-B2 | 91 | 101 | 91 | 93 | 101 | 91 | 74* | 97 | 68* |
| ERBB3 | 97 | 101 | 100 | 101 | 101 | 101 | 100 | 100 | 100 |
| P-B3 | 95 | 100 | 90 | 97 | 100 | 94 | 81* | 96 | 76* |
| ERBB4 | 101 | 99 | 99 | 100 | 98 | 98 | 99 | 100 | 100 |
| P-B4 | 94 | 102 | 93 | 100 | 97 | 94 | 75* | 96 | 73* |
| p70 S6K T389 | 89 | 92 | 86* | 102 | 99 | 91 | 87* | 99 | 85* |
| Total | 100 | 99 | 99 | 100 | 100 | 101 | 100 | 100 | 101 |
| ERK2 | | | | | | | | | |

SUPPLEMENTAL TABLE 2
The impact of GZ17-6.02 and osimertinib on cell signaling in NSCLC
cells (part 2). Cells were treated with vehicle control, GZ17-6.02 (2 mM final curcumin),
osimertinib (100 nM) or t he drugs combined for 6 h. Cells were fixed in place and immunostaining
performed to determine protein expression and phosphorylation (n = 3 +/− SD) * p < 0.05 less than
vehicle; ** p < 0.05 less than GZ17-6.02 alone; # p < 0.05 greater than vehicle.

| Vehicle | 1975 WT | | | 1975 AR | | | 1650 WT | | |
|---|---|---|---|---|---|---|---|---|---|
| 100% | 602 | OSI | 6 + O | 602 | OR | 6 + O | 602 | OSI | 6 + O |
| P-NFκB | 89 | 100 | 86* | 83* | 102 | 82* | 88 | 83* | 80* |
| S536 | | | | | | | | | |
| P-SRC | 83* | 95 | 81* | 86* | 95 | 76* | 86* | 83* | 80* |
| Y416 | | | | | | | | | |
| P-SRC | 105 | 102 | 107 | 107 | 103 | 109 | 107 | 105 | 107 |
| Y527 | | | | | | | | | |
| c-MET | 100 | 101 | 101 | 102 | 103 | 103 | 100 | 98 | 99 |
| P-c-MET | 88 | 92 | 87* | 89 | 93 | 85* | 92 | 94 | 87* |
| c-KIT | 98 | 98 | 98 | 102 | 102 | 102 | 93 | 100 | 93 |
| P-c-KIT | 91 | 99 | 91 | 98 | 109 | 118# | 85* | 92 | 79* |
| PDGFRβ | 100 | 100 | 91 | 98 | 98 | 98 | 103 | 106 | 103 |
| P- | 89 | 89 | 86* | 90 | 90 | 87* | 83* | 93 | 74* |
| PDGFRβ | | | | | | | | | |
| P-JAK2 | 86* | 83* | 83* | 81* | 86* | 81* | 83* | 86* | 75* |
| ERK2 | 99 | 99 | 99 | 100 | 101 | 100 | 99 | 100 | 100 |
| P-ERK1/2 | 87* | 70* | 61* [symbol] | 84* | 85* | 77* | 87* | 82* | 76* |
| JNK1/2 | 100 | 101 | 99 | 100 | 100 | 100 | 100 | 104 | 100 |
| P-JNK1/2 | 87 | 89 | 85* | 89 | 89 | 82* | 86* | 91 | 85* |
| CD95 | 100 | 102 | 100 | 106 | 104 | 105 | 99 | 100 | 99 |
| FAS-L | 102 | 101 | 110 | 101 | 100 | 99 | 107 | 104 | 111 |
| HDAC1 | 97 | 97 | 86* | 101 | 101 | 100 | 98 | 99 | 97 |
| HDAC2 | 79* | 99 | 68** | 73* | 98 | 71* | 75* | 98 | 68* |
| HDAC3 | 79* | 96 | 69* | 77* | 96 | 65** | 84* | 98 | 78* |
| HDAC4 | 88 | 93 | 80* | 87 | 103 | 81* | 85* | 101 | 84* |
| HDAC5 | 97 | 100 | 94 | 93 | 100 | 93 | 92 | 99 | 87* |
| HDAC6 | 80* | 105 | 69** | 75* | 103 | 60** | 76* | 98 | 74* |
| HDAC7 | 87 | 103 | 83* | 88 | 100 | 79* | 83* | 99 | 83* |
| HDAC8 | 96 | 99 | 92 | 96 | 100 | 95 | 97 | 101 | 94 |
| HDAC9 | 100 | 98 | 97 | 95 | 100 | 89 | 102 | 100 | 100 |
| HDAC10 | 96 | 99 | 89 | 97 | 97 | 100 | 100 | 98 | 97 |
| HDAC11 | 97 | 96 | 93 | 102 | 98 | 95 | 93 | 100 | 89 |
| PD-L1 | 84* | 99 | 74* | 82* | 99 | 74* | 86* | 97 | 80* |
| PD-L2 | 98 | 97 | 96 | 98 | 98 | 100 | 95 | 97 | 91 |
| MHCA | 114# | 96 | 118# | 120# | 99 | 119# | 112 | 103 | 116# |
| ODC | 90 | 100 | 89 | 90 | 101 | 90 | 95 | 104 | 93 |
| IDO1 | 89 | 99 | 85* | 91 | 100 | 86* | 92 | 100 | 87* |
| β-catenin | 102 | 98 | 95 | 102 | 99 | 96 | 100 | 101 | 100 |
| P-β- | 112 | 111 | 114* | 111 | 109 | 114* | 103 | 102 | 103 |
| catenin | | | | | | | | | |
| YAP | 99 | 99 | 100 | 98 | 99 | 100 | 99 | 98 | 97 |
| P-YAP | 112 | 113# | 116# | 124# | 124# | 126# | 116# | 102 | 117# |
| S127 | | | | | | | | | |
| TAZ | 104 | 100 | 100 | 102 | 102 | 100 | 100 | 101 | 98 |
| P-TAZ S89 | 112 | 114# | 115# | 119# | 122# | 124# | 113# | 101 | 116# |

SUPPLEMENTAL TABLE 3
The impact of GZ17-6.02 and pemetrexed on cell signaling in NSCLC
cells. Cells were treated wit h ve hicle control, GZ17-6.02 (2 mM final curcumin), pemetrexed (500
nM) or the drugs combined for 6 h. Cells were fixed in place and immunostaining performed to
determine protein expression and phosphorylation (n = 3 +/− SD) * p < 0.05 less than vehicle; #
p < 0.05 greater than vehicle.

| Vehicle | 1975 WT | | | 1975 AR | | | 1975 OR | | |
|---|---|---|---|---|---|---|---|---|---|
| 100% | 602 | PEM | 6 + P | 602 | PEM | 6 + P | 602 | PEM | 6 + P |
| P-ATM | 113# | 108 | 117# | 113# | 108 | 116# | 114# | 109 | 118# |
| S1980 | | | | | | | | | |
| P-AMPKα | 112 | 105 | 114# | 111 | 107 | 117# | 117# | 112 | 118# |
| T172 | | | | | | | | | |
| P-mTOR | 86* | 96 | 85* | 84* | 95 | 82* | 88 | 94 | 81* |
| S2448 | | | | | | | | | |
| P-mTOR | 78* | 91 | 75* | 81* | 83* | 78* | 79* | 92 | 75* |
| S2481 | | | | | | | | | |
| P-ULK1 | 85* | 91 | 82* | 89 | 95 | 82* | 89 | 96 | 82* |
| S757 | | | | | | | | | |
| P-ULK1 | 108 | 105 | 114# | 110 | 105 | 115# | 112 | 103 | 120# |
| S317 | | | | | | | | | |
| P-PERK | 112 | 103 | 116# | 114* | 102 | 118# | 118# | 101 | 122# |
| T980 | | | | | | | | | |
| P-eIF2α | 112 | 103 | 118# | 111 | 103 | 115# | 110 | 101 | 117# |
| S51 | | | | | | | | | |
| P-AKT | 84* | 87* | 79* | 87* | 88 | 84* | 83* | 87* | 79* |
| T308 | | | | | | | | | |
| P-STAT3 | 86* | 88 | 86* | 92 | 95 | 82* | 82* | 88 | 80* |
| Y705 | | | | | | | | | |
| P-STAT5 | 89 | 91 | 85* | 93 | 94 | 85* | 82* | 87* | 80* |
| Y694 | | | | | | | | | |
| Beclin1 | 115# | 112 | 117# | 112 | 111 | 118# | 115# | 111 | 120# |
| ATG5 | 115# | 109 | 117# | 112 | 107 | 117# | 115# | 110 | 119# |
| P-ATG13 | 112 | 104 | 117# | 114# | 105 | 118# | 115# | 106 | 120# |
| S318 | | | | | | | | | |
| GRP78 | 124# | 112 | 130# | 120# | 109 | 126# | 127# | 115# | 132# |
| CHOP | 115# | 107 | 117# | 112 | 104 | 116# | 114# | 107 | 119# |
| PP1 | 111 | 109 | 113# | 108 | 104 | 110 | 109 | 105 | 111 |
| ERBB1 | 100 | 101 | 98 | 100 | 101 | 100 | 100 | 99 | 100 |
| P-B1 | 85* | 88 | 75* | 87* | 93 | 78* | 94 | 88 | 84* |
| ERBB2 | 100 | 101 | 101 | 100 | 100 | 100 | 99 | 100 | 100 |
| P-B2 | 88 | 96 | 75* | 86* | 97 | 73* | 88 | 92 | 85* |
| ERBB3 | 99 | 99 | 99 | 99 | 99 | 100 | 100 | 101 | 100 |
| P-B3 | 89 | 95 | 85* | 91 | 97 | 80* | 90 | 93 | 86* |
| ERBB4 | 100 | 100 | 100 | 100 | 100 | 101 | 100 | 100 | 100 |
| P-B4 | 100 | 100 | 100 | 86* | 96 | 83* | 86* | 95 | 76* |
| p70 S6K | 100 | 101 | 102 | 100 | 100 | 100 | 100 | 101 | 100 |
| p70 S6K | 91 | 99 | 81* | 89 | 97 | 76* | 89 | 98 | 78* |
| T389 | | | | | | | | | |
| P-NFκB | 87* | 85* | 83* | 88 | 98 | 87* | 83* | 92 | 80* |
| S536 | | | | | | | | | |
| P-SRC | 95 | 94 | 94 | 86* | 92 | 85* | 92 | 90 | 86* |
| Y416 | | | | | | | | | |
| P-SRC | 105 | 109 | 108 | 107 | 109 | 113# | 107 | 112 | 113# |
| Y527 | | | | | | | | | |
| c-MET | 100 | 100 | 100 | 103 | 100 | 102 | 99 | 100 | 101 |
| P-c-MET | 94 | 94 | 90 | 93 | 102 | 93 | 84* | 88 | 82* |
| c-KIT | 99 | 99 | 100 | 100 | 101 | 100 | 100 | 101 | 102 |
| P-c-MT | 85* | 94 | 83* | 89 | 88 | 82* | 89 | 90 | 81* |
| PDGFRβ | 101 | 100 | 100 | 98 | 100 | 100 | 99 | 100 | 100 |
| P- | 85* | 90 | 82* | 82* | 84* | 80* | 81* | 98 | 79* |
| PDGFRβ | | | | | | | | | |
| JAK2 | 101 | 102 | 101 | 99 | 100 | 100 | 98 | 98 | 99 |
| P-JAK2 | 94 | 91 | 89 | 93 | 87* | 87* | 95 | 89 | 89 |
| ERK2 | 100 | 99 | 100 | 98 | 99 | 99 | 100 | 100 | 101 |
| P-ERK1/2 | 86* | 95 | 85* | 78* | 98 | 78* | 83* | 96 | 80* |
| CD95 | 97 | 100 | 100 | 101 | 101 | 100 | 101 | 100 | 100 |
| FAS-L | 104 | 108 | 113# | 103 | 108 | 113# | 105 | 110 | 116# |

SUPPLEMENTAL TABLE 4
The impact of GZ17-6.02 and pemetrexed on the expression
of cytoprotective proteins in NSCLC cells.

| | VEH | 602 | PEM | 6 + P |
|---|---|---|---|---|
| H1975 WT | | | | |
| FLIP-s | 100 | 97 | 101 | 93 |
| BCL-XL | 100 | 90 | 87* | 81* |
| MCL-1 | 100 | 85* | 81* | 80* |
| ERK2 | 100 | 100 | 100 | 100 |
| H1975 OR | | | | |
| FLIP-s | 100 | 99 | 98 | 98 |
| BCL-XL | 100 | 93 | 90 | 85* |
| MCL-1 | 100 | 89* | 84* | 82* |
| ERK2 | 100 | 101 | 101 | 101 |
| H1650 WT | | | | |
| FLIP-s | 100 | 91 | 92 | 88 |
| BCL-XL | 100 | 87* | 84* | 83* |
| MCL-1 | 100 | 86* | 83* | 80* |
| ERK2 | 100 | 100 | 100 | 101 |
| H1650 OR | | | | |
| FLIP-s | 100 | 97 | 93 | 91 |
| BCL-XL | 100 | 87* | 85* | 83* |
| MCL-1 | 100 | 90 | 86* | 83* |
| ERK2 | 100 | 101 | 100 | 100 |

Cells were treated with vehicle control, GZ17-6.02 (2 mM final curcumin), pemetrexed (500 nM) or the drugs combined for 6 h. Cells were fixed in place and immunostaining performed to determine protein expression
(n = 3 +/− SD) * p < 0.05 less than vehicle.

SUPPLEMENTAL TABLE 5
The impact of GZ17-6.02 and pemetrexed on the expression of histone
deacetylase (HDAC) proteins in NSCLC cells. Cells were treated wit h vehicle control, GZ17-
6.02 (2 mM final curcumin), pemetrexed (500 nM) or the drugs combined for 6 h. Cells were fixed
in place and immunostaining performed to determine protein expression (n = 3 +/− SD) * p < 0.05
less than vehicle.

| | 1975 WT | | | 1975 OR | | |
|---|---|---|---|---|---|---|
| | 602 | PEM | 6 + P | 602 | PEM | 6 + P |
| 1 | 94 | 99 | 86* | 96 | 97 | 97 |
| 2 | 92 | 93 | 86* | 90 | 93 | 84* |
| 3 | 91 | 100 | 84* | 93 | 98 | 85* |
| 4 | 100 | 100 | 101 | 97 | 100 | 98 |
| 5 | 99 | 98 | 94 | 94 | 95 | 90 |
| 6 | 82* | 88 | 81* | 84* | 93 | 83* |
| 7 | 94 | 98 | 94 | 95 | 98 | 97 |
| 8 | 100 | 101 | 102 | 100 | 101 | 100 |
| 9 | 101 | 99 | 100 | 96 | 97 | 96 |
| 10 | 102 | 102 | 98 | 100 | 100 | 102 |
| 11 | 101 | 101 | 101 | 98 | 101 | 100 |
| ERK2 | 100 | 100 | 100 | 102 | 101 | 102 |

SUPPLEMENTAL TABLE 6
The impact of GZ17-6.02 and pemetrexed on the expression of
immuno-regulatory proteins in NSCLC cells. Cells were treated with vehicle control, GZ17-
6.02 (2 mM final curcumin), pemetrexed (500 nM) or the drugs combined for 6 h. Cells were fixed
in place and immunostaining performed to determine protein expression (n = 3 +/− SD) * p < 0.05
less than vehicle; # p < 0.05 greater than vehicle.

| | A549 | | | H460 | | | H1437 | | | LLC | | | | | |
|---|---|---|---|---|---|---|---|---|---|---|---|---|---|---|---|
| | 602 | P | 6 + P | 602 | P | 6 + P | 602 | P | 6 + P | 602 | | P | | 6 + P | |
| PD-L1 | 64* | 85* | 65* | 66* | 87* | 69* | 78* | 85* | 72* | 68* | | 90 | | 65* | |
| PD-L2 | 89 | 102 | 99 | 97 | 104 | 93 | 97 | 102 | 97 | 95 | | 102 | | 94 | |
| MHCA | 126# | 111# | 126# | 125# | 112 | 128# | 125# | 112 | 129# | 123# | | 103 | | 127# | |
| IDO1 | 97 | 98 | 95 | 85* | 95 | 86* | 94 | 95 | 92 | 87 | | 97 | | 89 | |
| ODC | 90 | 98 | 87* | 105 | 103 | 91 | 93 | 96 | 85* | 95 | | 92 | | 91 | |
| ERK2 | 100 | 99 | 99 | 102 | 100 | 102 | 99 | 99 | 99 | 100 | | 98 | | 99 | |
| | HCC827ER | | | H661 | | | H573 | | | H1299 | | | | | |
| | 602 | P | 6 + P | 602 | P | 6 + P | 602 | P | 6 + P | 602 | | P | | 6 + P | |
| PD-L1 | 82* | 84* | 78* | 69* | 80* | 66* | 79* | 89 | 75* | 75* | | 84* | | 72* | |
| PD-L2 | 100 | 100 | 102 | 98 | 102 | 99 | 98 | 104 | 98 | 98 | | 100 | | 98 | |
| MHCA | 130# | 123# | 145## | 124# | 113# | 132# | 125# | 111 | 125# | 124# | | 109 | | 123# | |
| IDO1 | 69* | 78* | 70* | 75* | 83* | 74* | 82* | 92 | 84* | 87* | | 95 | | 86* | |
| ODC | 86* | 76* | 75* | 85* | 84* | 82* | 98 | 87* | 75** | 100 | | 99 | | 93 | |
| ERK2 | 99 | 100 | 100 | 101 | 100 | 100 | 101 | 102 | 101 | 100 | | 100 | | 100 | |
| | 1975WT | | | 1975AR | | | 1975OR | | | 1650WT | | | 1650OR | | |
| | 602 | P | 6 + P | 602 | P | 6 + P | 602 | P | 6 + P | 602 | P | 6 + P | 602 | P | 6 + P |
| PD-L1 | 72* | 86* | 71* | 84* | 89 | 73** | 90 | 96 | 82* | 74* | 85* | 71* | 86* | 94 | 84* |
| PD-L2 | 99 | 102 | 100 | 100 | 101 | 96 | 100 | 99 | 97 | 98 | 101 | 98 | 101 | 99 | 98 |
| MHCA | 127# | 110 | 125# | 128# | 108 | 136# | 115# | 112 | 122# | 124# | 109 | 123# | 116# | 107 | 118# |
| IDO1 | 84* | 86* | 82* | 80* | 91 | 78* | 91 | 93 | 83* | 87* | 88 | 86* | 90 | 100 | 86* |
| ODC | 89 | 89 | 80* | 81* | 80* | 84* | 89 | 93 | 82* | 100 | 91 | 93 | 88 | 89 | 85* |
| ERK2 | 100 | 100 | 101 | 100 | 100 | 99 | 101 | 100 | 100 | 100 | 99 | 100 | 99 | 101 | 100 |

1. A method of reducing or inhibiting the growth of cancer cells by treating said cancer cells with a therapeutic product comprising individual quantities of components a) and b), where component a) comprises individual amounts of curcumin, harmine, isovanillin, and component b) comprises an agent selected from the group consisting of EGFR inhibitors, CDK 4/6 inhibitors, checkpoint inhibitors, anti-metabolites, 5-fluorouracil, prodrugs thereof, and mixtures thereof.

2. The method of claim 1, the amount of said isovanillin being greater than the amounts of said harmine and said curcumin.

3. The method of claim 1, the weight ratio of curcumin:harmine:isovanillin in said product being from about 0.1-25.0:0.1-5:0.1-5.

4. The method of claim 1, said isovanillin present at a level of from about 25-85% by weight, said harmine present at a level of from about 7-50% by weight, and said curcumin being present at a level of from about 5-40% by weight, all based upon the total weight of said curcumin, harmine, and isovanillin taken as 100% by weight.

5. The method of claim 1, said agent comprising a checkpoint inhibitor targeting CTLA-4, PD-1, and/or PD-L1.

6. The method of claim 1, said agent comprising a checkpoint inhibitor selected from the group consisting of Ipilimumab, Nivolumab, Pembrolizumab, Atezolizumab, Avelumab, Durvalumab, Cemiplimab, and mixtures thereof.

7. The method of claim 1, said agent comprising an EGFR inhibitor targeting ERBB receptors.

8. The method of claim 7, said agent comprising an EGFR inhibitor selected from the group consisting of Erlotinib, Osimertinib, Gefitinb, Afatinib, Dacomitinib, Cetuximab, Panitumuab, Necitumumab, and mixtures thereof.

9. The method of claim 1, said agent comprising an anti-metabolite selected from 5-FU, Pemetrexed, and mixtures thereof.

10. The method of claim 1, said agent comprising a CDK 4/6 inhibitor selected from the group consisting of palbociclib, ribociclib, abemaciclib, and mixtures thereof.

11. The method of claim 1, said cancer cells being selected from the group consisting of skin, colorectal, breast, brain, lung, lymphatic system, rectal, colon, esophageal, cervical, stomach, and/or pancreatic cancer cells.

12. The method of claim 11, said product operable to reduce the expression of multiple HDAC proteins from said cancer cells.

13. The method of claim 1, comprising separately treating said cancer cells with said components a) and b).

14. The method of claim 13, comprising sequentially treating said cancer cells with said components a) and b).

15. The method of claim 1, for reducing or inhibiting the growth of cancer cells in a mammal comprising said cancer cells, wherein said treating step comprises administering a therapeutically-effective dosage of said therapeutic product to said mammal.

16. The method of claim 15, wherein said mammal comprises a tumor comprising said cancer cells.

17. The method of claim 16, wherein said tumor has a first size before administering said therapeutic product to said animal, and wherein said tumor has a second size after administering said therapeutic product to said mammal for a therapeutically-effective period of time, wherein said second size is smaller by volume than said first size.

18. The method of claim 15, wherein said product is administered orally.

19. The method of claim 15, wherein said product is administered intravenously.

20. An anti-cancer combination dosage form comprising individual quantities of components a) and b), where component a) comprises individual amounts of curcumin, harmine, isovanillin, and component b) comprises an agent selected from the group consisting of EGFR inhibitors, CDK 4/6 inhibitors, checkpoint inhibitors, anti-metabolites, 5-fluorouracil, prodrugs thereof, and mixtures thereof.

21. The combination dosage form of claim 20, the amount of said isovanillin being greater than the amounts of said harmine and said curcumin.

22. The combination dosage form of claim 20, the weight ratio of curcumin:harmine:isovanillin in said product being from about 0.1-25.0:0.1-5:0.1-5.

23. The combination dosage form of claim 20, said isovanillin present at a level of from about 25-85% by weight, said harmine present at a level of from about 7-50% by weight, and said curcumin being present at a level of from about 5-40% by weight, all based upon the total weight of said curcumin, harmine, and isovanillin taken as 100% by weight.

24. The combination dosage form of claim 20, said agent comprising a checkpoint inhibitor targeting CTLA-4, PD-1, and/or PD-L1.

25. The combination dosage form of claim 20, said agent comprising a checkpoint inhibitor selected from the group consisting of Ipilimumab, Nivolumab, Pembrolizumab, Atezolizumab, Avelumab, Durvalumab, Cemiplimab, and mixtures thereof.

26. The combination dosage form of claim 20, said agent comprising an EGFR inhibitor targeting ERBB receptors.

27. The combination dosage form of claim 20, said agent comprising an EGFR inhibitor selected from the group consisting of Erlotinib, Osimertinib, Gefitinb, Afatinib, Dacomitinib, Cetuximab, Panitumuab, Necitumumab, and mixtures thereof.

28. The combination dosage form of claim 20, said agent comprising an anti-metabolite selected from 5-FU, Pemetrexed, and mixtures thereof.

29. The combination dosage form of claim 20, said agent comprising a CDK 4/6 inhibitor selected from the group consisting of palbociclib, ribociclib, abemaciclib, and mixtures thereof.

30. A method of treating a mammalian subject suffering from cancer, comprising the step of administering to the subject the combination dosage form of claim 20.

31. The method of claim 30, said cancer being selected from the group consisting of skin, colorectal, breast, brain, lung, lymphatic system, rectal, colon, esophageal, cervical, stomach, and/or pancreatic cancer.
